(19) United States
(12) Patent Application Publication
(10) Pub. No.: US 2021/0076704 A1
(43) Pub. Date: Mar. 18, 2021

(54) ANIMAL FEED COMPOSITIONS AND USES THEREOF

(71) Applicant: DSM IP ASSETS B.V., Heerlen (NL)

(72) Inventors: Raffaella AURELI, Kaiseraugst (CH); Rual LOPEZ-ULIBARRI, Kaiseraugst (CH); Estefania PEREZ CALVO, Kaiseraugst (CH)

(21) Appl. No.: 16/954,383

(22) PCT Filed: Dec. 19, 2018
(86) PCT No.: PCT/EP2018/085863

§ 371 Date: Jun. 16, 2020

(30) Foreign Application Priority Data
Dec. 20, 2017 (EP) 17209027.6

(51) Int. Cl. A23K 20/147; A23K 10/35; A23K 20/189; A23K 20/174; A23K 20/20; A23K 40/10; A23K 50/75; A23K 50/30; A23K 10/18

(52) U.S. Cl. CPC A23K 20/147; A23K 10/35; A23K 20/189; A23K 20/174; A23K 10/18; A23K 40/10; A23K 50/75; A23K 50/30; A23K 20/20

(57) ABSTRACT

The present invention relates to animal feed compositions comprising polypeptides having muramidase activity and polypeptides having xylanase activity and uses thereof.

### REFERENCE TO A SEQUENCE LISTING

This application contains a Sequence Listing in computer readable form, which is incorporated herein by reference.

### BACKGROUND OF THE INVENTION

### Field of the Invention

The present invention relates to animal feed compositions comprising polypeptides having muramidase activity and polypeptides having xylanase activity and uses thereof.

### Description of the Related Art

Muramidase, also known as lysozyme, is an O-glycosyl hydrolase produced as a defensive mechanism against bacteria by many organisms. The enzyme causes the hydrolysis of bacterial cell walls by cleaving the glycosidic bonds of peptidoglycan; an important structural molecule in bacteria. After having their cell walls weakened by muramidase action, bacterial cells lyse as a result of unbalanced osmotic pressure.

Muramidase naturally occurs in many organisms such as viruses, plants, insects, birds, reptiles and mammals. In mammals, Muramidase has been isolated from nasal secretions, saliva, tears, intestinal content, urine and milk. The enzyme cleaves the glycosidic bond between carbon number 1 of N-acetylmuramic acid and carbon number 4 of N-acetyl-D-glucosamine. In vivo, these two carbohydrates are polymerized to form the cell wall polysaccharide of many microorganisms.

Muramidase has been classified into five different glycoside hydrolase (GH) families (CAZy, www.cazy.org): hen egg-white muramidase (GH22), goose egg-white muramidase (GH23), bacteriophage T4 muramidase (GH24), Sphingomonas flagellar protein (GH73) and Chalaropsis muramidases (GH25). Muramidase extracted from hen egg white (a GH22 muramidase) is the primary product available on the commercial market, and traditionally has just been referred to as muramidase even though nowadays there are many other known muramidases.

Xylans are hemicelluloses found in all land plants (Popper and Tuohy, Plant Physiology, 2010, 153:373-383). They are especially abundant in secondary cell walls and xylem cells. In grasses, with type II cell walls, glucurono arabinoxylans are the main hemicellulose and are present as soluble or insoluble dietary fiber in many grass based food and feed products.

The known enzymes responsible for the hydrolysis of the xylan backbone are classified into enzyme families based on sequence similarity (www.cazy.org). The enzymes with mainly endo-xylanase activity have previously been described in Glycoside hydrolase family (GH) 5, 8, 10, 11, 30 and 98. The enzymes within a family share some characteristics such as 3D fold and they usually share the same reaction mechanism. Some GH families have narrow or mono-specific substrate specificities while other families have broad substrate specificities.

Commercially available GH10 and GH11 xylanases are often used to break down the xylose backbone of arabinoxylan. In animal feed this results in a degradation of the cereal cell wall with a subsequent improvement in nutrient release (starch and protein) encapsulated within the cells. Degradation of xylan also results in the formation of xylose oligomers that may be utilised for hind gut fermentation and therefore can help an animal to obtain more digestible energy.

There are many patent applications disclosing the combination of a xylanase and a muramidase in combination with many other feed ingredients and/or herbal remedies. However, in all cases the muramidase tested is the muramidase from egg white and it is not possible to discern whether it is the combination of muramidase and xylanase alone which gives any benefit (if any) or the mixture of all the components.

It has been shown in WO 2017/001703 that microbial muramidases improve animal performance. However, combining different types of enzymes often doesn't result in any beneficial results over the single enzyme, see T. T. dos Santos et al., “Xylanase, protease and superdosing phytase interactions in broiler performance, carcass yield and digesta transit time”, Animal Nutrition (2017), 3, 121-126 and Adeola and Cowieson, “Opportunities and challenges in using exogenous enzymes to improve nonruminant animal production”, J Anim Sci, (2011), 89, 189-3218.

Improving the growth performance of farm animals is needed in a world with a growing population eating more animal protein, and it is the object of the present invention to devise solutions which helps meet this challenge.

### SUMMARY OF THE INVENTION

The present invention relates to an animal feed or animal feed additive comprising one or more polypeptides having xylanse activity and one or more polypeptides having muramidase activity.

The present invention further relates to a method of improving growth performance of an animal comprising administering to the animal an animal feed or animal feed additive comprising one or more polypeptides having xylanase activity and one or more polypeptides having muramidase activity.

The present invention further relates to methods of improving Body Weight Gain (BWG), Feed Conversion Ratio (FCR) and/or European Production Efficiency Factor (EPEF) of an animal comprising administering to the animal the animal feed or the animal feed additive of the invention; use of the animal feed or animal feed additive of the invention for improving Body Weight Gain (BWG), Feed Conversion Ratio (FCR) and/or European Production Efficiency Factor (EPEF) of an animal.

### Overview of Sequence Listing

SEQ ID NO: 1 is the mature amino acid sequence of a GH25 muramidase from Acremonium alcalophilum as described in WO2013/076253 (SEQ ID NO: 4).

SEQ ID NO: 2 is the mature amino acid sequence of a GH25 muramidase from Acremonium alcalophilum as described in WO2013/076253 (SEQ ID NO: 8).

SEQ ID NO: 3 is the mature amino acid sequence of a GH25 muramidase from Aspergillus fumigatus as described in WO2011/104339 (SEQ ID NO: 3).

SEQ ID NO: 4 is the mature amino acid sequence of a GH25 muramidase from Trichoderma reesei as described in WO2009/102755 (SEQ ID NO: 4).

SEQ ID NO: 5 is the mature amino acid sequence of a GH25 muramidase from Trametes cinnabarina as described in WO2005/080559 (SEQ ID NO: 2).

SEQ ID NO: 6 is the mature amino acid sequence of a GH25 muramidase from Sporormia fimetaria as described in PCT/CN2017/075978 (SEQ ID NO: 3).

SEQ ID NO: 7 is the mature amino acid sequence of a GH25 muramidase from Poronia punctata as described in PCT/CN2017/075978 (SEQ ID NO: 6).

SEQ ID NO: 8 is the mature amino acid sequence of a GH25 muramidase from Poronia punctata as described in PCT/CN2017/075978 (SEQ ID NO: 9).

SEQ ID NO: 9 is the mature amino acid sequence of a GH25 muramidase from Lecanicillium sp. WMM742 as described in PCT/CN2017/075978 (SEQ ID NO: 12).

SEQ ID NO: 10 is the mature amino acid sequence of a GH25 muramidase from Lecanicillium sp. WMM742 as described in PCT/CN2017/075978 (SEQ ID NO: 15).

SEQ ID NO: 11 is the mature amino acid sequence of a GH25 muramidase from Onygena equina as described in PCT/CN2017/075978 (SEQ ID NO: 18).

SEQ ID NO: 12 is the mature amino acid sequence of a GH25 muramidase from Purpureocillium lilacinum as described in PCT/CN2017/075978 (SEQ ID NO: 21).

SEQ ID NO: 13 is the mature amino acid sequence of a GH25 muramidase from Trichobolus zukalii as described in PCT/CN2017/075978 (SEQ ID NO: 24).

SEQ ID NO: 14 is the mature amino acid sequence of a GH25 muramidase from Penicillium citrinum as described in PCT/CN2017/075978 (SEQ ID NO: 27).

SEQ ID NO: 15 is the mature amino acid sequence of a GH25 muramidase from Cladorrhinum bulbillosum as described in PCT/CN2017/075978 (SEQ ID NO: 30).

SEQ ID NO: 16 is the mature amino acid sequence of a GH25 muramidase from Umbelopsis westeae as described in PCT/CN2017/075978 (SEQ ID NO: 33).

SEQ ID NO: 17 is the mature amino acid sequence of a GH25 muramidase from Zygomycetes sp. XZ2655 as described in PCT/CN2017/075978 (SEQ ID NO: 36).

SEQ ID NO: 18 is the mature amino acid sequence of a GH25 muramidase from Chaetomium cupreum as described in PCT/CN2017/075978 (SEQ ID NO: 39).

SEQ ID NO: 19 is the mature amino acid sequence of a GH25 muramidase from Cordyceps cardinalis as described in PCT/CN2017/075978 (SEQ ID NO: 42).

SEQ ID NO: 20 is the mature amino acid sequence of a GH25 muramidase from Penicillium sp. ‘qii’ as described in PCT/CN2017/075978 (SEQ ID NO: 45).

SEQ ID NO: 21 is the mature amino acid sequence of a GH25 muramidase from Aspergillus sp. nov XZ2609 as described in PCT/CN2017/075978 (SEQ ID NO: 48).

SEQ ID NO: 22 is the mature amino acid sequence of a GH25 muramidase from Paecilomyces sp. XZ2658 as described in PCT/CN2017/075978 (SEQ ID NO: 51).

SEQ ID NO: 23 is the mature amino acid sequence of a GH25 muramidase from Paecilomyces sp. XZ2658 as described in PCT/CN2017/075978 (SEQ ID NO: 54).

SEQ ID NO: 24 is the mature amino acid sequence of a GH25 muramidase from Pycnidiophora cf dispera as described in PCT/CN2017/075978 (SEQ ID NO: 60).

SEQ ID NO: 25 is the mature amino acid sequence of a GH25 muramidase from Thermomucor indicae-seudaticae as described in PCT/CN2017/075978 (SEQ ID NO: 63).

SEQ ID NO: 26 is the mature amino acid sequence of a GH25 muramidase from Isaria farinosa as described in PCT/CN2017/075978 (SEQ ID NO: 66).

SEQ ID NO: 27 is the mature amino acid sequence of a GH25 muramidase from Lecanicillium sp. WMM742 as described in PCT/CN2017/075978 (SEQ ID NO: 69).

SEQ ID NO: 28 is the mature amino acid sequence of a GH25 muramidase from Zopfiella sp. t180-6 as described in PCT/CN2017/075978 (SEQ ID NO: 72).

SEQ ID NO: 29 is the mature amino acid sequence of a GH25 muramidase from Malbranchea flava as described in PCT/CN2017/075978 (SEQ ID NO: 75).

SEQ ID NO: 30 is the mature amino acid sequence of a GH25 muramidase from Hypholoma polytrichi as described in PCT/CN2017/075978 (SEQ ID NO: 80).

SEQ ID NO: 31 is the mature amino acid sequence of a GH25 muramidase from Aspergillus deflectus as described in PCT/CN2017/075978 (SEQ ID NO: 83).

SEQ ID NO: 32 is the mature amino acid sequence of a GH25 muramidase from Ascobolus stictoideus as described in PCT/CN2017/075978 (SEQ ID NO: 86).

SEQ ID NO: 33 is the mature amino acid sequence of a GH25 muramidase from Coniochaeta sp. as described in PCT/CN2017/075978 (SEQ ID NO: 89).

SEQ ID NO: 34 is the mature amino acid sequence of a GH25 muramidase from Daldinia fissa as described in PCT/CN2017/075978 (SEQ ID NO: 92).

SEQ ID NO: 35 is the mature amino acid sequence of a GH25 muramidase from Roseffinia sp. as described in PCT/CN2017/075978 (SEQ ID NO: 95).

SEQ ID NO: 36 is the mature amino acid sequence of a GH25 muramidase from Ascobolus sp. ZY179 as described in PCT/CN2017/075978 (SEQ ID NO: 98).

SEQ ID NO: 37 is the mature amino acid sequence of a GH25 muramidase from Curreya sp. XZ2623 as described in PCT/CN2017/075978 (SEQ ID NO: 101).

SEQ ID NO: 38 is the mature amino acid sequence of a GH25 muramidase from Coniothyrium sp. as described in PCT/CN2017/075978 (SEQ ID NO: 104).

SEQ ID NO: 39 is the mature amino acid sequence of a GH25 muramidase from Hypoxylon sp. as described in PCT/CN2017/075978 (SEQ ID NO: 107).

SEQ ID NO: 40 is the mature amino acid sequence of a GH25 muramidase from Xylariaceae sp. 1653h as described in PCT/CN2017/075978 (SEQ ID NO: 110).

SEQ ID NO: 41 is the mature amino acid sequence of a GH25 muramidase from Hypoxylon sp. as described in PCT/CN2017/075978 (SEQ ID NO: 113).

SEQ ID NO: 42 is the mature amino acid sequence of a GH25 muramidase from Yunnania penicillata as described in PCT/CN2017/075978 (SEQ ID NO: 116).

SEQ ID NO: 43 is the mature amino acid sequence of a GH25 muramidase from Engyodontium album as described in PCT/CN2017/075978 (SEQ ID NO: 119).

SEQ ID NO: 44 is the mature amino acid sequence of a GH25 muramidase from Metapochonia bulbillosa as described in PCT/CN2017/075978 (SEQ ID NO: 122).

SEQ ID NO: 45 is the mature amino acid sequence of a GH25 muramidase from Hamigera paravellanea as described in PCT/CN2017/075978 (SEQ ID NO: 125).

SEQ ID NO: 46 is the mature amino acid sequence of a GH25 muramidase from Metarhizium iadini as described in PCT/CN2017/075978 (SEQ ID NO: 128).

SEQ ID NO: 47 is the mature amino acid sequence of a GH25 muramidase from Thermoascus aurantiacus as described in PCT/CN2017/075978 (SEQ ID NO: 131).

SEQ ID NO: 48 is the mature amino acid sequence of a GH25 muramidase from Clonostachys rossmaniae as described in PCT/CN2017/075978 (SEQ ID NO: 134).

SEQ ID NO: 49 is the mature amino acid sequence of a GH25 muramidase from Simplicillium obclavatum as described in PCT/CN2017/075978 (SEQ ID NO: 137).

SEQ ID NO: 50 is the mature amino acid sequence of a GH25 muramidase from Aspergillus inflatus as described in PCT/CN2017/075978 (SEQ ID NO: 140).

SEQ ID NO: 51 is the mature amino acid sequence of a GH25 muramidase from Paracremonium inflatum as described in PCT/CN2017/075978 (SEQ ID NO: 143).

SEQ ID NO: 52 is the mature amino acid sequence of a GH25 muramidase from Westerdykella sp. as described in PCT/CN2017/075978 (SEQ ID NO: 146).

SEQ ID NO: 53 is the mature amino acid sequence of a GH25 muramidase from Stropharia semiglobata as described in PCT/CN2017/075978 (SEQ ID NO: 155).

SEQ ID NO: 54 is the mature amino acid sequence of a GH25 muramidase from Gelasinospora cratophora as described in PCT/CN2017/075978 (SEQ ID NO: 158).

SEQ ID NO: 55 is the mature amino acid sequence of a GH25 muramidase from Flammulina velutipes as described in PCT/CN2017/075978 (SEQ ID NO: 221).

SEQ ID NO: 56 is the mature amino acid sequence of a GH25 muramidase from Deconica coprophila as described in PCT/CN2017/075978 (SEQ ID NO: 224).

SEQ ID NO: 57 is the mature amino acid sequence of a GH25 muramidase from Rhizomucor pusillus as described in PCT/CN2017/075978 (SEQ ID NO: 227).

SEQ ID NO: 58 is the mature amino acid sequence of a GH25 muramidase from Stropharia semiglobata as described in PCT/CN2017/075978 (SEQ ID NO: 230).

SEQ ID NO: 59 is the mature amino acid sequence of a GH25 muramidase from Stropharia semiglobata as described in PCT/CN2017/075978 (SEQ ID NO: 233).

SEQ ID NO: 60 is the mature amino acid sequence of a GH25 muramidase from Myceliophthora fergusii as described in PCT/CN2017/075960 (SEQ ID NO: 3).

SEQ ID NO: 61 is the mature amino acid sequence of a GH25 muramidase from Mortierella alpina as described in PCT/CN2017/075960 (SEQ ID NO: 15).

SEQ ID NO: 62 is the mature amino acid sequence of a GH25 muramidase from Penicillium atrovenetum as described in PCT/CN2017/075960 (SEQ ID NO: 27).

SEQ ID NO: 63 is the mature amino acid sequence of a GH24 muramidase from Trichophaea saccata as described in WO2017/000922 (SEQ ID NO: 257).

SEQ ID NO: 64 is the mature amino acid sequence of a GH24 muramidase from Chaetomium thermophilum as described in WO2017/000922 (SEQ ID NO: 264).

SEQ ID NO: 65 is the mature amino acid sequence of a GH24 muramidase from Trichoderma harzianum as described in WO2017/000922 (SEQ ID NO: 267).

SEQ ID NO: 66 is the mature amino acid sequence of a GH24 muramidase from Trichophaea minuta as described in WO2017/000922 (SEQ ID NO: 291).

SEQ ID NO: 67 is the mature amino acid sequence of a GH24 muramidase from Chaetomium sp. ZY287 as described in WO2017/000922 (SEQ ID NO: 294).

SEQ ID NO: 68 is the mature amino acid sequence of a GH24 muramidase from Mortierella sp. ZY002 as described in WO2017/000922 (SEQ ID NO: 297).

SEQ ID NO: 69 is the mature amino acid sequence of a GH24 muramidase from Metarhizium sp. XZ2431 as described in WO2017/000922 (SEQ ID NO: 300).

SEQ ID NO: 70 is the mature amino acid sequence of a GH24 muramidase from Geomyces auratus as described in WO2017/000922 (SEQ ID NO: 303).

SEQ ID NO: 71 is the mature amino acid sequence of a GH24 muramidase from Ilyonectria rufa as described in WO2017/000922 (SEQ ID NO: 306).

SEQ ID NO: 72 is the mature amino acid sequence of a GH10 xylanase from Aspergillus aculeatus as described in WO1994/021785 (SEQ ID NO: 5).

SEQ ID NO: 73 is the mature amino acid sequence of a GH10 xylanase from Clostridium acetobutylicum as described in Appl. Environ. Microbiol. 1987, 53(4):644.

SEQ ID NO: 74 is the mature amino acid sequence of a GH10 xylanase from Aspergillus aculeatus as described in WO2005/059084 (SEQ ID NO: 8).

SEQ ID NO: 75 is the mature amino acid sequence of a GH10 xylanase from Thermotoga maritima MSB8 as described in WO2013/068550 (SEQ ID NO: 1).

SEQ ID NO: 76 is the mature amino acid sequence of a GH10 xylanase from Ascobolus stictoideus as described in WO2016/095856 (SEQ ID NO: 102).

SEQ ID NO: 77 is the mature amino acid sequence of a GH10 xylanase from Ustilago maydis. as described in WO2016/095856 (SEQ ID NO: 177).

SEQ ID NO: 78 is the mature amino acid sequence of a GH10 xylanase from Talaromyces emersonii as described in WO2001/42433 (SEQ ID NO: 1).

SEQ ID NO: 79 is the mature amino acid sequence of a GH10 xylanase from Talaromyces emersonii as described in WO2002/24926 (SEQ ID NO: 2).

SEQ ID NO: 80 is the mature amino acid sequence of a GH11 xylanase from Myceliophthora thermophila as described in WO2009/018537 (SEQ ID NO: 41).

SEQ ID NO: 81 is the mature amino acid sequence of a GH11 xylanase from Lasiodiplodia theobromae as described in WO2016/095856 (SEQ ID NO: 99).

SEQ ID NO: 82 is the mature amino acid sequence of a GH11 xylanase from Penicillium funiculosum as described in WO1999/57325 (SEQ ID NO: 1).

SEQ ID NO: 83 is the mature amino acid sequence of a GH11 xylanase from Bacillus subtilis as described in WO2001/66711 (SEQ ID NO: 1).

SEQ ID NO: 84 is the mature amino acid sequence of a GH11 xylanase from Trichoderma viride as described in WO2002/38746 (FIG. 16G).

SEQ ID NO: 85 is the mature amino acid sequence of a GH11 xylanase from Thermopolyspora flexuosa as described in WO2005100557 (SEQ ID NO: 12).

SEQ ID NO: 86 is the mature amino acid sequence of a GH11 xylanase from Trichoderma reesei as described in WO1993/24621 (SEQ ID NO: 2).

SEQ ID NO: 87 is the mature amino acid sequence of a GH11 xylanase from Trichoderma reesei as described in WO1993/24621 (SEQ ID NO: 4).

SEQ ID NO: 88 is the mature amino acid sequence of a GH11 xylanase from Bacillus subtilis as described in U.S. Pat. No. 5,306,633 (SEQ ID NO: 3).

SEQ ID NO: 89 is the mature amino acid sequence of a GH11 xylanase from Penicillium funiculosum as described in WO2007/146944 (SEQ ID NO: 79).

SEQ ID NO: 90 is the mature amino acid sequence of a GH11 xylanase from Thermomyces lanuginosus as described in WO2003/062409 (SEQ ID NO: 2).

SEQ ID NO: 91 is the mature amino acid sequence of a GH11 xylanase from Dictyoglomus thermophilum as described in WO2011/057140 (SEQ ID NO: 305).

SEQ ID NO: 92 is the mature amino acid sequence of a GH11 xylanase from Paenibacillus Pabuli as described in WO2005/079585 (SEQ ID NO: 2).

SEQ ID NO: 93 is the mature amino acid sequence of a GH11 xylanase from Geobacillus stearothermophilus as described in WO2016/095856 (SEQ ID NO: 78).

SEQ ID NO: 94 is the mature amino acid sequence of a GH11 xylanase from Streptomyces beijiangensis as described in WO2016/095856 (SEQ ID NO: 84).

SEQ ID NO: 95 is the mature amino acid sequence of a GH11 xylanase from Fusarium oxysporum as described in WO2014/019220 (SEQ ID NO: 8).

SEQ ID NO: 96 is the mature amino acid sequence of a GH11 xylanase from Aspergillus clavatus as described in WO2014/020143 (SEQ ID NO: 8).

SEQ ID NO: 97 is the mature amino acid sequence of a GH5 xylanase from Paenibacillus illinoisensis as described in WO2016/005522 (SEQ ID NO: 3).

SEQ ID NO: 98 is the mature amino acid sequence of a GH5 xylanase from Paenibacillus sp-18054 as described in WO2016/005522 (SEQ ID NO: 9).

SEQ ID NO: 99 is the mature amino acid sequence of a GH5 xylanase from elephant dung metagenome as described in WO2016/005522 (SEQ ID NO: 15).

SEQ ID NO: 100 is the mature amino acid sequence of a GH5 xylanase from Chryseobacterium sp-10696 as described in WO2016/005522 (SEQ ID NO: 27).

SEQ ID NO: 101 is the mature amino acid sequence of a GH5 xylanase from elephant dung metagenome as described in WO2016/005522 (SEQ ID NO: 39).

SEQ ID NO: 102 is the mature amino acid sequence of a GH5 xylanase from elephant dung metagenome as described in WO2016/005522 (SEQ ID NO: 45).

SEQ ID NO: 103 is the mature amino acid sequence of a GH5 xylanase from Paenibacillus campinasensis as described in WO2016/005522 (SEQ ID NO: 67).

SEQ ID NO: 104 is the mature amino acid sequence of a GH5 xylanase from Paenibacillus sp-62250 as described in WO2016/005522 (SEQ ID NO: 73).

SEQ ID NO: 105 is the mature amino acid sequence of a GH5 xylanase from Paenibacillus favisporus as described in WO2016/005522 (SEQ ID NO: 79).

SEQ ID NO: 106 is the mature amino acid sequence of a GH5 xylanase from Paenibacillus tundrae as described in WO2016/005522 (SEQ ID NO: 85).

SEQ ID NO: 107 is the mature amino acid sequence of a GH5 xylanase from Paenibacillus sp-62603 as described in WO2016/005522 (SEQ ID NO: 91).

SEQ ID NO: 108 is the mature amino acid sequence of a GH5 xylanase from Paenibacillus sp-62332 as described in WO2016/005522 (SEQ ID NO: 103).

SEQ ID NO: 109 is the mature amino acid sequence of a GH5 xylanase from Paenibacillus sp-62248 as described in WO2016/005522 (SEQ ID NO: 109).

SEQ ID NO: 110 is the mature amino acid sequence of a GH5 xylanase from compost metagenome as described in WO2016/005522 (SEQ ID NO: 127).

SEQ ID NO: 111 is the mature amino acid sequence of a GH30 xylanase from Bacillus subtilis as described in PCT/EP2017/065336 (SEQ ID NO: 1).

SEQ ID NO: 112 is the mature amino acid sequence of a GH30 xylanase from Bacillus amyloliquefaciens as described in PCT/EP2017/065336 (SEQ ID NO: 2).

SEQ ID NO: 113 is the mature amino acid sequence of a GH30 xylanase from Bacillus licheniformis as described in PCT/EP2017/065336 (SEQ ID NO: 3).

SEQ ID NO: 114 is the mature amino acid sequence of a GH30 xylanase from Bacillus subtilis as described in PCT/EP2017/065336 (SEQ ID NO: 4).

SEQ ID NO: 115 is the mature amino acid sequence of a GH30 xylanase from Paenibacillus pabuli as described in PCT/EP2017/065336 (SEQ ID NO: 5).

SEQ ID NO: 116 is the mature amino acid sequence of a GH30 xylanase from Bacillus amyloliquefaciens HB-26 as described in PCT/EP2017/065336 (SEQ ID NO: 6).

SEQ ID NO: 117 is the mature amino acid sequence of a GH30 xylanase from Pseudoalteromonas tetraodonis. as described in WO2017/103159 (SEQ ID NO: 6).

SEQ ID NO: 118 is the mature amino acid sequence of a GH30 xylanase from Paenibacillus sp-19179. as described in WO2017/103159 (SEQ ID NO: 12).

SEQ ID NO: 119 is the mature amino acid sequence of a GH30 xylanase from Pectobacterium carotovorum subsp. carotovorum as described in WO2017/103159 (SEQ ID NO: 18).

SEQ ID NO: 120 is the mature amino acid sequence of a GH30 xylanase from Ruminococcus sp. CAG:330 as described in WO2017/103159 (SEQ ID NO: 24).

SEQ ID NO: 121 is the mature amino acid sequence of a GH30 xylanase from Streptomyces sp-62627. as described in WO2017/103159 (SEQ ID NO: 30).

SEQ ID NO: 122 is the mature amino acid sequence of a GH30 xylanase from Clostridium saccharobutylicum as described in WO2017/103159 (SEQ ID NO: 36).

SEQ ID NO: 123 is the mature amino acid sequence of a GH30 xylanase from Paenibacillus panacisoli as described in WO2017/103159 (SEQ ID NO: 42).

SEQ ID NO: 124 is the mature amino acid sequence of a GH30 xylanase from Human Stool metagenome. as described in WO2017/103159 (SEQ ID NO: 48).

SEQ ID NO: 125 is the mature amino acid sequence of a GH30 xylanase from Vibrio rhizosphaerae. as described in WO2017/103159 (SEQ ID NO: 54).

SEQ ID NO: 126 is the mature amino acid sequence of a GH30 xylanase from Clostridium acetobutylicum. as described in WO2017/103159 (SEQ ID NO: 60).

### Definitions

Animal: The term “animal” refers to any animal except humans. Examples of animals are monogastric animals, including but not limited to pigs or swine (including, but not limited to, piglets, growing pigs, and sows); poultry such as turkeys, ducks, quail, guinea fowl, geese, pigeons (including squabs) and chicken (including but not limited to broiler chickens (referred to herein as broiles), chicks, layer hens (referred to herein as layers)); pet animals such as cats and dogs; horses (including but not limited to hotbloods, coldbloods and warm bloods) crustaceans (including but not limited to shrimps and prawns) and fish (including but not limited to amberjack, arapaima, barb, bass, bluefish, bocachico, bream, bullhead, cachama, carp, catfish, catla, chanos, char, cichlid, cobia, cod, crappie, dorada, drum, eel, goby, goldfish, gourami, grouper, guapote, halibut, Java, labeo, lai, loach, mackerel, milkfish, mojarra, mudfish, mullet, paco, pearlspot, pejerrey, perch, pike, pompano, roach, salmon, sampa, sauger, sea bass, seabream, shiner, sleeper, snakehead, snapper, snook, sole, spinefoot, sturgeon, sunfish, sweetfish, tench, terror, tilapia, trout, tuna, turbot, vendace, walleye and whitefish).

Animal feed: The term “animal feed” refers to any compound, preparation, or mixture suitable for, or intended for intake by an animal. Animal feed for a monogastric animal typically comprises concentrates as well as vitamins, minerals, enzymes, direct fed microbial, amino acids and/or other feed ingredients (such as in a premix) whereas animal feed for ruminants generally comprises forage (including roughage and silage) and may further comprise concentrates as well as vitamins, minerals, enzymes direct fed microbial, amino acid and/or other feed ingredients (such as in a premix).

Body Weight Gain: The term “body weight gain” means an increase in live weight of an animal during a given period of time e.g. the increase in weight from day 1 to day 21.

Concentrates: The term “concentrates” means feed with high protein and energy concentrations, such as fish meal, molasses, oligosaccharides, sorghum, seeds and grains (either whole or prepared by crushing, milling, etc. from e.g. corn, oats, rye, barley, wheat), oilseed press cake (e.g. from cottonseed, safflower, sunflower, soybean (such as soybean meal), rapeseed/canola, peanut or groundnut), palm kernel cake, yeast derived material and distillers grains (such as wet distillers grains (WDS) and dried distillers grains with solubles (DDGS)).

Feed Conversion Ratio (FCR): FCR is a measure of an animal's efficiency in converting feed mass into increases of the desired output. Animals raised for meat—such as swine, poultry and fish—the output is the mass gained by the animal. Specifically FCR is calculated as feed intake divided by body weight gain, all over a specified period. Improvement in FCR means reduction of the FCR value. A FCR improvement of 2% means that the FCR was reduced by 2%.

European Production Efficiency Factor (EPEF): The European Production Efficiency Factor is a way of comparing the performance of animals. This single-figure facilitates comparison of performance within and among farms and can be used to assess environmental, climatic and animal management variables. The EPEF is calculated as [(liveability (%)×Liveweight (kg))/(Age at depletion (days)×FCR)]×100, wherein livability is the percentage of animals alive at slaughter, Liveweight is the average weight of the animals at slaughter, age of depletion is the age of the animals at slaughter and FCR is the feed conversion ratio at slaughter.

Feed efficiency: The term “feed efficiency” means the amount of weight gain per unit of feed when the animal is fed ad-libitum or a specified amount of food during a period of time. By “increased feed efficiency” it is meant that the use of a feed additive composition according the present invention in feed results in an increased weight gain per unit of feed intake compared with an animal fed without said feed additive composition being present.

Forage: The term “forage” as defined herein also includes roughage. Forage is fresh plant material such as hay and silage from forage plants, grass and other forage plants, seaweed, sprouted grains and legumes, or any combination thereof. Examples of forage plants are Alfalfa (lucerne), birdsfoot trefoil, brassica (e.g. kale, rapeseed (canola), rutabaga (swede), turnip), clover (e.g. alsike clover, red clover, subterranean clover, white clover), grass (e.g. Bermuda grass, brome, false oat grass, fescue, heath grass, meadow grasses, orchard grass, ryegrass, Timothy-grass), corn (maize), millet, barley, oats, rye, sorghum, soybeans and wheat and vegetables such as beets. Forage further includes crop residues from grain production (such as corn stover; straw from wheat, barley, oat, rye and other grains); residues from vegetables like beet tops; residues from oilseed production like stems and leaves form soy beans, rapeseed and other legumes; and fractions from the refining of grains for animal or human consumption or from fuel production or other industries.

Fragment: The term “fragment” means a polypeptide or a catalytic domain having one or more (e.g., several) amino acids absent from the amino and/or carboxyl terminus of a mature polypeptide or domain; wherein the fragment has muramidase or xylanase activity.

In one aspect, a fragment of a GH24 muramidase (such as one of SEQ ID NO: 63 to 71) comprises at least 230 amino acids, such as at least 235 amino acids, at least 240 amino acids, or at least 245 amino acids and has muramidase activity. In another aspect, a fragment of a GH24 muramidase (such as one of SEQ ID NO: 63 to 71) comprises at least 90% of the length of the mature polypeptide, such as at least 92%, at least 94%, at least 96%, at least 98% or at least 99% of the length of the mature polypeptide and has muramidase activity.

In one aspect, a fragment of a GH25 muramidase (such as one of SEQ ID NO: 1 to 72) comprises at least 180 amino acids, such as at least 185 amino acids, at least 190 amino acids, at least 195 amino acids, at least 200 amino acids, at least 205 amino acids or at least 210 amino acids and has muramidase activity. In another aspect, a fragment of a GH25 muramidase (such as one of SEQ ID NO: 1 to 72) comprises at least 90% of the length of the mature polypeptide, such as at least 92%, at least 94%, at least 96%, at least 98% or at least 99% of the length of the mature polypeptide and has muramidase activity.

In one aspect, a fragment of a GH10 xylanase (such as one of SEQ ID NO: 72 to 79) comprises at least 90% of the length of the mature polypeptide, such as at least 92%, at least 94%, at least 96%, at least 98% or at least 99% of the length of the mature polypeptide and has muramidase activity.

In one aspect, a fragment of a GH11 xylanase (such as one of SEQ ID NO: 80 to 96) comprises at least 90% of the length of the mature polypeptide, such as at least 92%, at least 94%, at least 96%, at least 98% or at least 99% of the length of the mature polypeptide and has muramidase activity.

In one aspect, a fragment of a GH5 xylanase (such as one of SEQ ID NO: 97 to 110) comprises at least 90% of the length of the mature polypeptide, such as at least 92%, at least 94%, at least 96%, at least 98% or at least 99% of the length of the mature polypeptide and has muramidase activity.

In one aspect, a fragment of a GH30 xylanase (such as one of SEQ ID NO: 111 to 126) comprises at least 90% of the length of the mature polypeptide, such as at least 92%, at least 94%, at least 96%, at least 98% or at least 99% of the length of the mature polypeptide and has muramidase activity.

Isolated: The term “isolated” means a substance in a form or environment that does not occur in nature. Non-limiting examples of isolated substances include (1) any non-naturally occurring substance, (2) any substance including, but not limited to, any enzyme, variant, nucleic acid, protein, peptide or cofactor, that is at least partially removed from one or more or all of the naturally occurring constituents with which it is associated in nature; (3) any substance modified by the hand of man relative to that substance found in nature; or (4) any substance modified by increasing the amount of the substance relative to other components with which it is naturally associated (e.g., multiple copies of a gene encoding the substance; use of a stronger promoter than the promoter naturally associated with the gene encoding the substance). An isolated substance may be present in a fermentation broth sample.

Muramidase activity: The term “muramidase activity” means the enzymatic hydrolysis of the 1,4-beta-linkages between N-acetylmuramic acid and N-acetyl-D-glucosamine residues in a peptidoglycan or between N-acetyl-D-glucosamine residues in chitodextrins, resulting in bacteriolysis due to osmotic pressure. Muramidase belongs to the enzyme class EC 3.2.1.17. Muramidase activity is typically measured by turbidimetric determination. The method is based on the changes in turbidity of a suspension of Micrococcus luteus ATCC 4698 induced by the lytic action of muramidase. In appropriate experimental conditions these changes are proportional to the amount of muramidase in the medium (c.f. INS 1105 of the Combined Compendium of Food Additive Specifications of the Food and Agriculture Organisation of the UN (www.fao.org)). For the purpose of the present invention, muramidase activity is determined according to the turbidity assay described in example 3 (“Determination of Muramidase Activity”) and the polypeptide has muramidase activity if it shows activity against one or more bacteria, such as Micrococcus luteus ATCC 4698 and/or Exiguobacterium undea (DSM14481). In one aspect, the GH25 muramidase of the present invention has at least 20%, e.g., at least 40%, at least 50%, at least 60%, at least 70%, at least 80%, at least 90%, at least 95%, or at least 100% of the muramidase activity of SEQ ID NO: 1. In one aspect, the GH24 muramidase of the present invention have at least 20%, e.g., at least 40%, at least 50%, at least 60%, at least 70%, at least 80%, at least 90%, at least 95%, or at least 100% of the muramidase activity of SEQ ID NO: 63.

Mature polypeptide: The term “mature polypeptide” means a polypeptide in its final form following translation and any post-translational modifications, such as N-terminal processing, C-terminal truncation, glycosylation, phosphorylation, etc.

In one aspect, the mature polypeptide is amino acids 1 to 208 of SEQ ID NO: 1. In one aspect, the mature polypeptide is amino acids 1 to 213 of SEQ ID NO: 2. In one aspect, the mature polypeptide is amino acids 1 to 218 of SEQ ID NO: 3. In one aspect, the mature polypeptide is amino acids 1 to 208 of SEQ ID NO: 4. In one aspect, the mature polypeptide is amino acids 1 to 215 of SEQ ID NO: 5. In one aspect, the mature polypeptide is amino acids 1 to 207 of SEQ ID NO: 6. In one aspect, the mature polypeptide is amino acids 1 to 201 of SEQ ID NO: 7. In one aspect, the mature polypeptide is amino acids 1 to 201 of SEQ ID NO: 8. In one aspect, the mature polypeptide is amino acids 1 to 203 of SEQ ID NO: 9. In one aspect, the mature polypeptide is amino acids 1 to 208 of SEQ ID NO: 10. In one aspect, the mature polypeptide is amino acids 1 to 207 of SEQ ID NO: 11. In one aspect, the mature polypeptide is amino acids 1 to 208 of SEQ ID NO: 12. In one aspect, the mature polypeptide is amino acids 1 to 207 of SEQ ID NO: 13. In one aspect, the mature polypeptide is amino acids 1 to 207 of SEQ ID NO: 14. In one aspect, the mature polypeptide is amino acids 1 to 207 of SEQ ID NO: 15. In one aspect, the mature polypeptide is amino acids 1 to 208 of SEQ ID NO: 16. In one aspect, the mature polypeptide is amino acids 1 to 208 of SEQ ID NO: 17. In one aspect, the mature polypeptide is amino acids 1 to 206 of SEQ ID NO: 18. In one aspect, the mature polypeptide is amino acids 1 to 207 of SEQ ID NO: 19. In one aspect, the mature polypeptide is amino acids 1 to 216 of SEQ ID NO: 20. In one aspect, the mature polypeptide is amino acids 1 to 218 of SEQ ID NO: 21. In one aspect, the mature polypeptide is amino acids 1 to 204 of SEQ ID NO: 22. In one aspect, the mature polypeptide is amino acids 1 to 203 of SEQ ID NO: 23. In one aspect, the mature polypeptide is amino acids 1 to 208 of SEQ ID NO: 24. In one aspect, the mature polypeptide is amino acids 1 to 210 of SEQ ID NO: 25. In one aspect, the mature polypeptide is amino acids 1 to 207 of SEQ ID NO: 26. In one aspect, the mature polypeptide is amino acids 1 to 207 of SEQ ID NO: 27. In one aspect, the mature polypeptide is amino acids 1 to 208 of SEQ ID NO: 28. In one aspect, the mature polypeptide is amino acids 1 to 217 of SEQ ID NO: 29. In one aspect, the mature polypeptide is amino acids 1 to 208 of SEQ ID NO: 30. In one aspect, the mature polypeptide is amino acids 1 to 201 of SEQ ID NO: 31. In one aspect, the mature polypeptide is amino acids 1 to 202 of SEQ ID NO: 32. In one aspect, the mature polypeptide is amino acids 1 to 207 of SEQ ID NO: 33. In one aspect, the mature polypeptide is amino acids 1 to 202 of SEQ ID NO: 34. In one aspect, the mature polypeptide is amino acids 1 to 201 of SEQ ID NO: 35. In one aspect, the mature polypeptide is amino acids 1 to 202 of SEQ ID NO: 36. In one aspect, the mature polypeptide is amino acids 1 to 206 of SEQ ID NO: 37. In one aspect, the mature polypeptide is amino acids 1 to 202 of SEQ ID NO: 38. In one aspect, the mature polypeptide is amino acids 1 to 202 of SEQ ID NO: 39. In one aspect, the mature polypeptide is amino acids 1 to 202 of SEQ ID NO: 40. In one aspect, the mature polypeptide is amino acids 1 to 202 of SEQ ID NO: 41. In one aspect, the mature polypeptide is amino acids 1 to 206 of SEQ ID NO: 42. In one aspect, the mature polypeptide is amino acids 1 to 207 of SEQ ID NO: 43. In one aspect, the mature polypeptide is amino acids 1 to 208 of SEQ ID NO: 44. In one aspect, the mature polypeptide is amino acids 1 to 215 of SEQ ID NO: 45. In one aspect, the mature polypeptide is amino acids 1 to 217 of SEQ ID NO: 46. In one aspect, the mature polypeptide is amino acids 1 to 214 of SEQ ID NO: 47. In one aspect, the mature polypeptide is amino acids 1 to 208 of SEQ ID NO: 48. In one aspect, the mature polypeptide is amino acids 1 to 203 of SEQ ID NO: 49. In one aspect, the mature polypeptide is amino acids 1 to 216 of SEQ ID NO: 50. In one aspect, the mature polypeptide is amino acids 1 to 207 of SEQ ID NO: 51. In one aspect, the mature polypeptide is amino acids 1 to 208 of SEQ ID NO: 52. In one aspect, the mature polypeptide is amino acids 1 to 207 of SEQ ID NO: 53. In one aspect, the mature polypeptide is amino acids 1 to 208 of SEQ ID NO: 54. In one aspect, the mature polypeptide is amino acids 1 to 207 of SEQ ID NO: 55. In one aspect, the mature polypeptide is amino acids 1 to 207 of SEQ ID NO: 56. In one aspect, the mature polypeptide is amino acids 1 to 208 of SEQ ID NO: 57. In one aspect, the mature polypeptide is amino acids 1 to 207 of SEQ ID NO: 58. In one aspect, the mature polypeptide is amino acids 1 to 207 of SEQ ID NO: 59. In one aspect, the mature polypeptide is amino acids 1 to 207 of SEQ ID NO: 60. In one aspect, the mature polypeptide is amino acids 1 to 204 of SEQ ID NO: 61. In one aspect, the mature polypeptide is amino acids 1 to 216 of SEQ ID NO: 62. In one aspect, the mature polypeptide is amino acids 1 to 245 of SEQ ID NO: 63. In one aspect, the mature polypeptide is amino acids 1 to 249 of SEQ ID NO: 64. In one aspect, the mature polypeptide is amino acids 1 to 248 of SEQ ID NO: 65. In one aspect, the mature polypeptide is amino acids 1 to 245 of SEQ ID NO: 66. In one aspect, the mature polypeptide is amino acids 1 to 249 of SEQ ID NO: 67. In one aspect, the mature polypeptide is amino acids 1 to 245 of SEQ ID NO: 68. In one aspect, the mature polypeptide is amino acids 1 to 247 of SEQ ID NO: 69. In one aspect, the mature polypeptide is amino acids 1 to 250 of SEQ ID NO: 70. In one aspect, the mature polypeptide is amino acids 1 to 240 of SEQ ID NO: 71. In one aspect, the mature polypeptide is amino acids 1 to 384 of SEQ ID NO: 72. In one aspect, the mature polypeptide is amino acids 1 to 288 of SEQ ID NO: 73. In one aspect, the mature polypeptide is amino acids 1 to 308 of SEQ ID NO: 74. In one aspect, the mature polypeptide is amino acids 1 to 328 of SEQ ID NO: 75. In one aspect, the mature polypeptide is amino acids 1 to 337 of SEQ ID NO: 76. In one aspect, the mature polypeptide is amino acids 1 to 323 of SEQ ID NO: 77. In one aspect, the mature polypeptide is amino acids 1 to 381 of SEQ ID NO: 78. In one aspect, the mature polypeptide is amino acids 1 to 386 of SEQ ID NO: 79. In one aspect, the mature polypeptide is amino acids 1 to 208 of SEQ ID NO: 80. In one aspect, the mature polypeptide is amino acids 1 to 203 of SEQ ID NO: 81. In one aspect, the mature polypeptide is amino acids 1 to 206 of SEQ ID NO: 82. In one aspect, the mature polypeptide is amino acids 1 to 185 of SEQ ID NO: 83. In one aspect, the mature polypeptide is amino acids 1 to 190 of SEQ ID NO: 84. In one aspect, the mature polypeptide is amino acids 1 to 220 of SEQ ID NO: 85. In one aspect, the mature polypeptide is amino acids 1 to 204 of SEQ ID NO: 86. In one aspect, the mature polypeptide is amino acids 1 to 210 of SEQ ID NO: 87. In one aspect, the mature polypeptide is amino acids 1 to 185 of SEQ ID NO: 88. In one aspect, the mature polypeptide is amino acids 1 to 264 of SEQ ID NO: 89. In one aspect, the mature polypeptide is amino acids 1 to 195 of SEQ ID NO: 90. In one aspect, the mature polypeptide is amino acids 1 to 203 of SEQ ID NO: 91. In one aspect, the mature polypeptide is amino acids 1 to 182 of SEQ ID NO: 92. In one aspect, the mature polypeptide is amino acids 1 to 183 of SEQ ID NO: 93. In one aspect, the mature polypeptide is amino acids 1 to 299 of SEQ ID NO: 94. In one aspect, the mature polypeptide is amino acids 1 to 188 of SEQ ID NO: 95. In one aspect, the mature polypeptide is amino acids 1 to 189 of SEQ ID NO: 96. In one aspect, the mature polypeptide is amino acids 1 to 537 of SEQ ID NO: 97. In one aspect, the mature polypeptide is amino acids 1 to 547 of SEQ ID NO: 98. In one aspect, the mature polypeptide is amino acids 1 to 598 of SEQ ID NO: 99. In one aspect, the mature polypeptide is amino acids 1 to 550 of SEQ ID NO: 100. In one aspect, the mature polypeptide is amino acids 1 to 828 of SEQ ID NO: 101. In one aspect, the mature polypeptide is amino acids 1 to 577 of SEQ ID NO: 102. In one aspect, the mature polypeptide is amino acids 1 to 537 of SEQ ID NO: 103. In one aspect, the mature polypeptide is amino acids 1 to 536 of SEQ ID NO: 104. In one aspect, the mature polypeptide is amino acids 1 to 536 of SEQ ID NO: 105. In one aspect, the mature polypeptide is amino acids 1 to 535 of SEQ ID NO: 106. In one aspect, the mature polypeptide is amino acids 1 to 536 of SEQ ID NO: 107. In one aspect, the mature polypeptide is amino acids 1 to 536 of SEQ ID NO: 108. In one aspect, the mature polypeptide is amino acids 1 to 536 of SEQ ID NO: 109. In one aspect, the mature polypeptide is amino acids 1 to 536 of SEQ ID NO: 110. In one aspect, the mature polypeptide is amino acids 1 to 391 of SEQ ID NO: 111. In one aspect, the mature polypeptide is amino acids 1 to 391 of SEQ ID NO: 112. In one aspect, the mature polypeptide is amino acids 1 to 392 of SEQ ID NO: 113. In one aspect, the mature polypeptide is amino acids 1 to 391 of SEQ ID NO: 114. In one aspect, the mature polypeptide is amino acids 1 to 393 of SEQ ID NO: 115. In one aspect, the mature polypeptide is amino acids 1 to 391 of SEQ ID NO: 116. In one aspect, the mature polypeptide is amino acids 1 to 382 of SEQ ID NO: 117. In one aspect, the mature polypeptide is amino acids 1 to 391 of SEQ ID NO: 118. In one aspect, the mature polypeptide is amino acids 1 to 383 of SEQ ID NO: 119. In one aspect, the mature polypeptide is amino acids 1 to 565 of SEQ ID NO: 120. In one aspect, the mature polypeptide is amino acids 1 to 396 of SEQ ID NO: 121. In one aspect, the mature polypeptide is amino acids 1 to 392 of SEQ ID NO: 122. In one aspect, the mature polypeptide is amino acids 1 to 413 of SEQ ID NO: 123. In one aspect, the mature polypeptide is amino acids 1 to 398 of SEQ ID NO: 124. In one aspect, the mature polypeptide is amino acids 1 to 372 of SEQ ID NO: 125. In one aspect, the mature polypeptide is amino acids 1 to 557 of SEQ ID NO: 126.

Obtained or obtainable from: The term “obtained or obtainable from” means that the polypeptide may be found in an organism from a specific taxonomic rank. In one embodiment, the polypeptide is obtained or obtainable from the kingdom Fungi, wherein the term kingdom is the taxonomic rank. In a preferred embodiment, the polypeptide is obtained or obtainable from the phylum Ascomycota, wherein the term phylum is the taxonomic rank. In another preferred embodiment, the polypeptide is obtained or obtainable from the subphylum Pezizomycotina, wherein the term subphylum is the taxonomic rank. In another preferred embodiment, the polypeptide is obtained or obtainable from the class Eurotiomycetes, wherein the term class is the taxonomic rank.

If the taxonomic rank of a polypeptide is not known, it can easily be determined by a person skilled in the art by performing a BLASTP search of the polypeptide (using e.g. the National Center for Biotechnology Information (NCIB) website http://www.ncbi.nlm.nih.gov/) and comparing it to the closest homologues. The skilled person can also compare the sequence to those of the application as filed. An unknown polypeptide which is a fragment of a known polypeptide is considered to be of the same taxonomic species. An unknown natural polypeptide or artificial variant which comprises a substitution, deletion and/or insertion in up to 10 positions is considered to be from the same taxonomic species as the known polypeptide.

Roughage: The term “roughage” means dry plant material with high levels of fiber, such as fiber, bran, husks from seeds and grains and crop residues (such as stover, copra, straw, chaff, sugar beet waste).

Sequence identity: The relatedness between two amino acid sequences or between two nucleotide sequences is described by the parameter “sequence identity”.

For purposes of the present invention, the sequence identity between two amino acid sequences is determined using the Needleman-Wunsch algorithm (Needleman and Wunsch, 1970, J. Mol. Biol. 48: 443-453) as implemented in the Needle program of the EMBOSS package (EMBOSS: The European Molecular Biology Open Software Suite, Rice et al., 2000, Trends Genet. 16: 276-277), preferably version 5.0.0 or later. The parameters used are gap open penalty of 10, gap extension penalty of 0.5, and the EBLOSUM62 (EMBOSS version of BLOSUM62) substitution matrix. The output of Needle labeled “longest identity” (obtained using the—nobrief option) is used as the percent identity and is calculated as follows:

(Identical Residues×100)/(Length of Alignment−Total Number of Gaps in Alignment)

Substantially pure polypeptide: The term “substantially pure polypeptide” means a preparation that contains at most 10%, at most 8%, at most 6%, at most 5%, at most 4%, at most 3%, at most 2%, at most 1%, and at most 0.5% by weight of other polypeptide material with which it is natively or recombinantly associated. Preferably, the polypeptide is at least 92% pure, e.g., at least 94% pure, at least 95% pure, at least 96% pure, at least 97% pure, at least 98% pure, at least 99%, at least 99.5% pure, and 100% pure by weight of the total polypeptide material present in the preparation. The polypeptides of the present invention are preferably in a substantially pure form. This can be accomplished, for example, by preparing the polypeptide by well known recombinant methods or by classical purification methods.

Variant: The term “variant” means a polypeptide having muramidase or xylanase activity comprising an alteration, i.e., a substitution, insertion, and/or deletion, of one or more (several) amino acid residues at one or more (e.g., several) positions. A substitution means replacement of the amino acid occupying a position with a different amino acid; a deletion means removal of the amino acid occupying a position; and an insertion means adding 1, 2, or 3 amino acids adjacent to and immediately following the amino acid occupying the position.

In one aspect, a muramidase variant may comprise from 1 to 10 alterations, i.e. 1, 2, 3, 4, 5, 6, 7, 8, 9 or 10 alterations and have at least 20%, e.g., at least 40%, at least 50%, at least 60%, at least 70%, at least 80%, at least 90%, at least 95%, or at least 100% of the muramidase activity of the parent muramidase, such as SEQ ID NO: 1 or SEQ ID NO: 63.

In one aspect, a xylanase variant may comprise from 1 to 10 alterations, i.e. 1, 2, 3, 4, 5, 6, 7, 8, 9 or 10 alterations and have at least 20%, e.g., at least 40%, at least 50%, at least 60%, at least 70%, at least 80%, at least 90%, at least 95%, or at least 100% of the xylanase activity of the parent xylanase, such as SEQ ID NO: 72, SEQ ID NO: 80, SEQ ID NO: 97 or SEQ ID NO: 111.

Xylanase: The term “xylanase” means a glucuronoarabinoxylan endo-1,4-beta-xylanase (E.C. 3.2.1.136) that catalyses the endohydrolysis of 1,4-beta-D-xylosyl links in some glucuronoarabinoxylans. Xylanase activity can be determined with 0.2% AZCL-glucuronoxylan as substrate in 0.01% TRITON® X-100 and 200 mM sodium phosphate pH 6 at 37° C. One unit of xylanase activity is defined as 1.0 μmole of azurine produced per minute at 37° C., pH 6 from 0.2% AZCL-glucuronoxylan as substrate in 200 mM sodium phosphate pH 6. The term “xylanase” also means a 1,4-beta-D-xylan-xylohydrolase (E.C. 3.2.1.8) that catalyses the endohydrolysis of 1,4-beta-D-xylosidic linkages in xylans. Xylanase activity can be determined with 0.2% AZCL-arabinoxylan as substrate in 0.01% TRITON® X-100 and 200 mM sodium phosphate pH 6 at 37° C. One unit of xylanase activity is defined as 1.0 μmole of azurine produced per minute at 37° C., pH 6 from 0.2% AZCL-arabinoxylan as substrate in 200 mM sodium phosphate pH 6.

### DETAILED DESCRIPTION OF THE INVENTION

### Animal Feed Comprising Polypeptides Having Muramidase Activity and Polypeptides Having Xylanase Activity

It has been surprisingly found that supplementing an animal feed comprising an animal feed additive, one or more protein sources and one or more energy sources with a muramidase (preferably a fungal muramidase) and a xylanase gives an additional performance benefit in animals compared to the same animal feed but without muramidase and xylanase present.

Thus, in a first aspect, the invention relates to an animal feed comprising an animal feed additive, one or more protein sources and one or more energy sources characterised in the animal feed or the animal feed additive further comprises one or more polypeptides having xylanase activity and one or more fungal polypeptides having muramidase activity.

In one embodiment, the muramidase is a GH24 muramidase, preferably a fungal GH24 muramidase, preferably obtained or obtainable from the phylum Ascomycota, more preferably from the class Eurotiomycetes. In one embodiment, the muramidase is a GH25 muramidase, preferably a fungal GH25 muramidase, preferably obtained or obtainable from the phylum Ascomycota, more preferably from the class Eurotiomycetes. In one embodiment, the xylanase is a GH10 xylanase. In one embodiment, the xylanase is a GH11 xylanase. In one embodiment, the xylanase is a GH5 xylanase, preferably a GH5 subfamily 21 or 35 xylanase (herein written GH5_21 and GH5_35 respectively). In one embodiment, the xylanase is a GH30 xylanase, preferably a GH30 subfamily 8 xylanase (herein written GH30_8).

In one embodiment, the muramidase is a GH24 muramidase, preferably a fungal GH24 muramidase, and the xylanase is a GH10 xylanase. In one embodiment, the muramidase is a GH24 muramidase, preferably a fungal GH24 muramidase, and the xylanase is a GH11 xylanase. In one embodiment, the muramidase is a GH24 muramidase, preferably a fungal GH24 muramidase, and the xylanase is a GH5 xylanase. In one embodiment, the muramidase is a GH24 muramidase, preferably a fungal GH24 muramidase, and the xylanase is a GH5_21 xylanase. In one embodiment, the muramidase is a GH24 muramidase, preferably a fungal GH24 muramidase, and the xylanase is a GH5_35 xylanase. In one embodiment, the muramidase is a GH24 muramidase, preferably a fungal GH24 muramidase, and the xylanase is a GH30 xylanase. In one embodiment, the muramidase is a GH24 muramidase, preferably a fungal GH24 muramidase, and the xylanase is a GH30_8 xylanase. In one embodiment, the muramidase is obtained or obtainable from the phylum Ascomycota, more preferably from the class Eurotiomycetes.

In one embodiment, the muramidase is a GH25 muramidase, preferably a fungal GH25 muramidase, and the xylanase is a GH10 xylanase. In one embodiment, the muramidase is a GH25 muramidase, preferably a fungal GH25 muramidase, and the xylanase is a GH11 xylanase. In one embodiment, the muramidase is a GH25 muramidase, preferably a fungal GH25 muramidase, and the xylanase is a GH5 xylanase. In one embodiment, the muramidase is a GH25 muramidase, preferably a fungal GH25 muramidase, and the xylanase is a GH5_21 xylanase. In one embodiment, the muramidase is a GH25 muramidase, preferably a fungal GH25 muramidase, and the xylanase is a GH5_35 xylanase. In one embodiment, the muramidase is a GH25 muramidase, preferably a fungal GH25 muramidase, and the xylanase is a GH30 xylanase. In one embodiment, the muramidase is a GH25 muramidase, preferably a fungal GH25 muramidase, and the xylanase is a GH30_8 xylanase. In one embodiment, the muramidase is obtained or obtainable from the phylum Ascomycota, more preferably from the class Eurotiomycetes.

In one embodiment, the muramidase is a fungal GH24 muramidase that degrades cell wall debris from Lactobacillus johnsonii and the xylanase is a GH10 xylanase. In one embodiment, the muramidase is a fungal GH24 muramidase that degrades cell wall debris from Lactobacillus johnsonii and the xylanase is a GH11 xylanase. In one embodiment, the muramidase is a fungal GH24 muramidase that degrades cell wall debris from Lactobacillus johnsonii and the xylanase is a GH5 xylanase. In one embodiment, the muramidase is a fungal GH24 muramidase that degrades cell wall debris from Lactobacillus johnsonii and the xylanase is a GH5_21 xylanase. In one embodiment, the muramidase is a fungal GH24 muramidase that degrades cell wall debris from Lactobacillus johnsonii and the xylanase is a GH5_35 xylanase. In one embodiment, the muramidase is a fungal GH24 muramidase that degrades cell wall debris from Lactobacillus johnsonii and the xylanase is a GH30 xylanase. In one embodiment, the muramidase is a fungal GH24 muramidase that degrades cell wall debris from Lactobacillus johnsonii and the xylanase is a GH30_8 xylanase. In one embodiment, the muramidase is obtained or obtainable from the phylum Ascomycota, more preferably from the class Eurotiomycetes.

In one embodiment, the muramidase is a fungal GH25 muramidase that degrades cell wall debris from Lactobacillus johnsonii and the xylanase is a GH10 xylanase. In one embodiment, the muramidase is a fungal GH25 muramidase that degrades cell wall debris from Lactobacillus johnsonii and the xylanase is a GH11 xylanase. In one embodiment, the muramidase is a fungal GH25 muramidase that degrades cell wall debris from Lactobacillus johnsonii and the xylanase is a GH5 xylanase. In one embodiment, the muramidase is a fungal GH25 muramidase that degrades cell wall debris from Lactobacillus johnsonii and the xylanase is a GH5_21 xylanase. In one embodiment, the muramidase is a fungal GH25 muramidase that degrades cell wall debris from Lactobacillus johnsonii and the xylanase is a GH5_35 xylanase. In one embodiment, the muramidase is a fungal GH25 muramidase that degrades cell wall debris from Lactobacillus johnsonii and the xylanase is a GH30 xylanase. In one embodiment, the muramidase is a fungal GH25 muramidase that degrades cell wall debris from Lactobacillus johnsonii and the xylanase is a GH30_8 xylanase. In one embodiment, the muramidase is obtained or obtainable from the phylum Ascomycota, more preferably from the class Eurotiomycetes.

In one embodiment, the invention relates to an animal feed comprising an animal feed additive, one or more protein sources and one or more energy sources characterised in that the animal feed or the animal feed additive further comprises one or more polypeptides having xylanase activity and one or more fungal polypeptides having muramidase activity, wherein the fungal polypeptide having muramidase activity activity is selected from the group consisting of:
(a) a polypeptide having at least 80%, e.g., at least 85%, at least 90%, at least 95%, or 100% sequence identity to SEQ ID NO: 1;
(b) a polypeptide having at least 80%, e.g., at least 85%, at least 90%, at least 95%, or 100% sequence identity to SEQ ID NO: 2;
(c) a polypeptide having at least 80%, e.g., at least 85%, at least 90%, at least 95%, or 100% sequence identity to SEQ ID NO: 3;
(d) a polypeptide having at least 80%, e.g., at least 85%, at least 90%, at least 95%, or 100% sequence identity to SEQ ID NO: 4;
(e) a polypeptide having at least 80%, e.g., at least 85%, at least 90%, at least 95%, or 100% sequence identity to SEQ ID NO: 5;
(f) a polypeptide having at least 80%, e.g., at least 85%, at least 90%, at least 95%, or 100% sequence identity to SEQ ID NO: 6;
(g) a polypeptide having at least 80%, e.g., at least 85%, at least 90%, at least 95%, or 100% sequence identity to SEQ ID NO: 7;
(h) a polypeptide having at least 80%, e.g., at least 85%, at least 90%, at least 95%, or 100% sequence identity to SEQ ID NO: 8;
(i) a polypeptide having at least 80%, e.g., at least 85%, at least 90%, at least 95%, or 100% sequence identity to SEQ ID NO: 9;
(j) a polypeptide having at least 80%, e.g., at least 85%, at least 90%, at least 95%, or 100% sequence identity to SEQ ID NO: 10;
(k) a polypeptide having at least 80%, e.g., at least 85%, at least 90%, at least 95%, or 100% sequence identity to SEQ ID NO: 11;
(l) a polypeptide having at least 80%, e.g., at least 85%, at least 90%, at least 95%, or 100% sequence identity to SEQ ID NO: 12;
(m) a polypeptide having at least 80%, e.g., at least 85%, at least 90%, at least 95%, or 100% sequence identity to SEQ ID NO: 13;
(n) a polypeptide having at least 80%, e.g., at least 85%, at least 90%, at least 95%, or 100% sequence identity to SEQ ID NO: 14;
(o) a polypeptide having at least 80%, e.g., at least 85%, at least 90%, at least 95%, or 100% sequence identity to SEQ ID NO: 15;
(p) a polypeptide having at least 80%, e.g., at least 85%, at least 90%, at least 95%, or 100% sequence identity to SEQ ID NO: 16;
(q) a polypeptide having at least 80%, e.g., at least 85%, at least 90%, at least 95%, or 100% sequence identity to SEQ ID NO: 17;
(r) a polypeptide having at least 80%, e.g., at least 85%, at least 90%, at least 95%, or 100% sequence identity to SEQ ID NO: 18;
(s) a polypeptide having at least 80%, e.g., at least 85%, at least 90%, at least 95%, or 100% sequence identity to SEQ ID NO: 19;
(t) a polypeptide having at least 80%, e.g., at least 85%, at least 90%, at least 95%, or 100% sequence identity to SEQ ID NO: 20;
(u) a polypeptide having at least 80%, e.g., at least 85%, at least 90%, at least 95%, or 100% sequence identity to SEQ ID NO: 21;
(v) a polypeptide having at least 80%, e.g., at least 85%, at least 90%, at least 95%, or 100% sequence identity to SEQ ID NO: 22;
(w) a polypeptide having at least 80%, e.g., at least 85%, at least 90%, at least 95%, or 100% sequence identity to SEQ ID NO: 23;
(x) a polypeptide having at least 80%, e.g., at least 85%, at least 90%, at least 95%, or 100% sequence identity to SEQ ID NO: 24;
(y) a polypeptide having at least 80%, e.g., at least 85%, at least 90%, at least 95%, or 100% sequence identity to SEQ ID NO: 25;
(z) a polypeptide having at least 80%, e.g., at least 85%, at least 90%, at least 95%, or 100% sequence identity to SEQ ID NO: 26;
(aa) a polypeptide having at least 80%, e.g., at least 85%, at least 90%, at least 95%, or 100% sequence identity to SEQ ID NO: 27;
(ab) a polypeptide having at least 80%, e.g., at least 85%, at least 90%, at least 95%, or 100% sequence identity to SEQ ID NO: 28;
(ac) a polypeptide having at least 80%, e.g., at least 85%, at least 90%, at least 95%, or 100% sequence identity to SEQ ID NO: 29;
(ad) a polypeptide having at least 80%, e.g., at least 85%, at least 90%, at least 95%, or 100% sequence identity to SEQ ID NO: 30;
(ae) a polypeptide having at least 80%, e.g., at least 85%, at least 90%, at least 95%, or 100% sequence identity to SEQ ID NO: 31;
(af) a polypeptide having at least 80%, e.g., at least 85%, at least 90%, at least 95%, or 100% sequence identity to SEQ ID NO: 32;
(ag) a polypeptide having at least 80%, e.g., at least 85%, at least 90%, at least 95%, or 100% sequence identity to SEQ ID NO: 33;
(ah) a polypeptide having at least 80%, e.g., at least 85%, at least 90%, at least 95%, or 100% sequence identity to SEQ ID NO: 34;
(ai) a polypeptide having at least 80%, e.g., at least 85%, at least 90%, at least 95%, or 100% sequence identity to SEQ ID NO: 35;
(aj) a polypeptide having at least 80%, e.g., at least 85%, at least 90%, at least 95%, or 100% sequence identity to SEQ ID NO: 36;
(ak) a polypeptide having at least 80%, e.g., at least 85%, at least 90%, at least 95%, or 100% sequence identity to SEQ ID NO: 37;
(al) a polypeptide having at least 80%, e.g., at least 85%, at least 90%, at least 95%, or 100% sequence identity to SEQ ID NO: 38;
(am) a polypeptide having at least 80%, e.g., at least 85%, at least 90%, at least 95%, or 100% sequence identity to SEQ ID NO: 39;
(an) a polypeptide having at least 80%, e.g., at least 85%, at least 90%, at least 95%, or 100% sequence identity to SEQ ID NO: 40;
(ao) a polypeptide having at least 80%, e.g., at least 85%, at least 90%, at least 95%, or 100% sequence identity to SEQ ID NO: 41;
(ap) a polypeptide having at least 80%, e.g., at least 85%, at least 90%, at least 95%, or 100% sequence identity to SEQ ID NO: 42;
(aq) a polypeptide having at least 80%, e.g., at least 85%, at least 90%, at least 95%, or 100% sequence identity to SEQ ID NO: 43;
(ar) a polypeptide having at least 80%, e.g., at least 85%, at least 90%, at least 95%, or 100% sequence identity to SEQ ID NO: 44;
(as) a polypeptide having at least 80%, e.g., at least 85%, at least 90%, at least 95%, or 100% sequence identity to SEQ ID NO: 45;
(at) a polypeptide having at least 80%, e.g., at least 85%, at least 90%, at least 95%, or 100% sequence identity to SEQ ID NO: 46;
(au) a polypeptide having at least 80%, e.g., at least 85%, at least 90%, at least 95%, or 100% sequence identity to SEQ ID NO: 47;
(av) a polypeptide having at least 80%, e.g., at least 85%, at least 90%, at least 95%, or 100% sequence identity to SEQ ID NO: 48;
(aw) a polypeptide having at least 80%, e.g., at least 85%, at least 90%, at least 95%, or 100% sequence identity to SEQ ID NO: 49;
(ax) a polypeptide having at least 80%, e.g., at least 85%, at least 90%, at least 95%, or 100% sequence identity to SEQ ID NO: 50;
(ay) a polypeptide having at least 80%, e.g., at least 85%, at least 90%, at least 95%, or 100% sequence identity to SEQ ID NO: 51;
(az) a polypeptide having at least 80%, e.g., at least 85%, at least 90%, at least 95%, or 100% sequence identity to SEQ ID NO: 52;
(ba) a polypeptide having at least 80%, e.g., at least 85%, at least 90%, at least 95%, or 100% sequence identity to SEQ ID NO: 53;
(bb) a polypeptide having at least 80%, e.g., at least 85%, at least 90%, at least 95%, or 100% sequence identity to SEQ ID NO: 54;
(bc) a polypeptide having at least 80%, e.g., at least 85%, at least 90%, at least 95%, or 100% sequence identity to SEQ ID NO: 55;
(bd) a polypeptide having at least 80%, e.g., at least 85%, at least 90%, at least 95%, or 100% sequence identity to SEQ ID NO: 56;
(be) a polypeptide having at least 80%, e.g., at least 85%, at least 90%, at least 95%, or 100% sequence identity to SEQ ID NO: 57;
(bf) a polypeptide having at least 80%, e.g., at least 85%, at least 90%, at least 95%, or 100% sequence identity to SEQ ID NO: 58;
(bg) a polypeptide having at least 80%, e.g., at least 85%, at least 90%, at least 95%, or 100% sequence identity to SEQ ID NO: 59;
(bh) a polypeptide having at least 80%, e.g., at least 85%, at least 90%, at least 95%, or 100% sequence identity to SEQ ID NO: 60;
(bi) a polypeptide having at least 80%, e.g., at least 85%, at least 90%, at least 95%, or 100% sequence identity to SEQ ID NO: 61;
(bj) a polypeptide having at least 80%, e.g., at least 85%, at least 90%, at least 95%, or 100% sequence identity to SEQ ID NO: 62;
(bk) a polypeptide having at least 80%, e.g., at least 85%, at least 90%, at least 95%, or 100% sequence identity to SEQ ID NO: 63;
(bl) a polypeptide having at least 80%, e.g., at least 85%, at least 90%, at least 95%, or 100% sequence identity to SEQ ID NO: 64;
(bm) a polypeptide having at least 80%, e.g., at least 85%, at least 90%, at least 95%, or 100% sequence identity to SEQ ID NO: 65;
(bn) a polypeptide having at least 80%, e.g., at least 85%, at least 90%, at least 95%, or 100% sequence identity to SEQ ID NO: 66;
(bo) a polypeptide having at least 80%, e.g., at least 85%, at least 90%, at least 95%, or 100% sequence identity to SEQ ID NO: 67;
(bp) a polypeptide having at least 80%, e.g., at least 85%, at least 90%, at least 95%, or 100% sequence identity to SEQ ID NO: 68;
(bq) a polypeptide having at least 80%, e.g., at least 85%, at least 90%, at least 95%, or 100% sequence identity to SEQ ID NO: 69;
(br) a polypeptide having at least 80%, e.g., at least 85%, at least 90%, at least 95%, or 100% sequence identity to SEQ ID NO: 70;
(bs) a polypeptide having at least 80%, e.g., at least 85%, at least 90%, at least 95%, or 100% sequence identity to SEQ ID NO: 71;
(bt) a variant of SEQ ID NO: 1, SEQ ID NO: 2, SEQ ID NO: 3, SEQ ID NO: 4, SEQ ID NO: 5, SEQ ID NO: 6, SEQ ID NO: 7, SEQ ID NO: 8, SEQ ID NO: 9, SEQ ID NO: 10, SEQ ID NO: 11, SEQ ID NO: 12, SEQ ID NO: 13, SEQ ID NO: 14, SEQ ID NO: 15, SEQ ID NO: 16, SEQ ID NO: 17, SEQ ID NO: 18, SEQ ID NO: 19, SEQ ID NO: 20, SEQ ID NO: 21, SEQ ID NO: 22, SEQ ID NO: 23, SEQ ID NO: 24, SEQ ID NO: 25, SEQ ID NO: 26, SEQ ID NO: 27, SEQ ID NO: 28, SEQ ID NO: 29, SEQ ID NO: 30, SEQ ID NO: 31, SEQ ID NO: 32, SEQ ID NO: 33, SEQ ID NO: 34, SEQ ID NO: 35, SEQ ID NO: 36, SEQ ID NO: 37, SEQ ID NO: 38, SEQ ID NO: 39, SEQ ID NO: 40, SEQ ID NO: 41, SEQ ID NO: 42, SEQ ID NO: 43, SEQ ID NO: 44, SEQ ID NO: 45, SEQ ID NO: 46, SEQ ID NO: 47, SEQ ID NO: 48, SEQ ID NO: 49, SEQ ID NO: 50, SEQ ID NO: 51, SEQ ID NO: 52, SEQ ID NO: 53, SEQ ID NO: 54, SEQ ID NO: 55, SEQ ID NO: 56, SEQ ID NO: 57, SEQ ID NO: 58, SEQ ID NO: 59, SEQ ID NO: 60, SEQ ID NO: 61, SEQ ID NO: 62, SEQ ID NO: 63, SEQ ID NO: 64, SEQ ID NO: 65, SEQ ID NO: 66, SEQ ID NO: 67, SEQ ID NO: 68, SEQ ID NO: 69, SEQ ID NO: 70 or SEQ ID NO: 71 comprising one or more amino acid substitutions (preferably conservative substitutions), and/or one or more amino acid deletions, and/or one or more amino acid insertions or any combination thereof in 1, 2, 3, 4, 5, 6, 7, 8, 9 or 10 positions;
(bu) a polypeptide comprising the polypeptide of (a), (b), (c), (d), (e), (f), (g), (h), (i), (j), (k), (l), (m), (n), (o), (p), (q), (r), (s), (t), (u), (v), (w), (x), (y), (z), (aa), (ab), (ac), (ad), (ae), (af), (ag), (ah), (ai), (aj), (ak), (al), (am), (an), (ao), (ap), (aq), (ar), (as), (at), (au), (av), (aw), (ax), (ay), (az), (ba), (bb), (bc), (bd), (be), (bf), (bg), (bh), (bi), (bj), (bk), (bl), (bm), (bn), (bo), (bp), (bq), (br), (bs) or (bt) and a N-terminal and/or C-terminal extension of between 1 and 10 amino acids; and
(bv) a fragment of a polypeptide of (a), (b), (c), (d), (e), (f), (g), (h), (i), (j), (k), (l), (m), (n), (o), (p), (q), (r), (s), (t), (u), (v), (w), (x), (y), (z), (aa), (ab), (ac), (ad), (ae), (af), (ag), (ah), (ai), (aj), (ak), (al), (am), (an), (ao), (ap), (aq), (ar), (as), (at), (au), (av), (aw), (ax), (ay), (az), (ba), (bb), (bc), (bd), (be), (bf), (bg), (bh), (bi), (bj), (bk), (bl), (bm), (bn), (bo), (bp), (bq), (br), (bs) or (bt) having muramidase activity and having at least 90% of the length of the mature polypeptide.

In one embodiment, the muramidase comprises or consists of amino acids 1 to 208 of SEQ ID NO: 1, amino acids 1 to 213 of SEQ ID NO: 2, amino acids 1 to 218 of SEQ ID NO: 3, amino acids 1 to 208 of SEQ ID NO: 4, amino acids 1 to 215 of SEQ ID NO: 5, amino acids 1 to 207 of SEQ ID NO: 6, amino acids 1 to 201 of SEQ ID NO: 7, amino acids 1 to 201 of SEQ ID NO: 8, amino acids 1 to 203 of SEQ ID NO: 9, amino acids 1 to 208 of SEQ ID NO: 10, amino acids 1 to 207 of SEQ ID NO: 11, amino acids 1 to 208 of SEQ ID NO: 12, amino acids 1 to 207 of SEQ ID NO: 13, amino acids 1 to 207 of SEQ ID NO: 14, amino acids 1 to 207 of SEQ ID NO: 15, amino acids 1 to 208 of SEQ ID NO: 16, amino acids 1 to 208 of SEQ ID NO: 17, amino acids 1 to 206 of SEQ ID NO: 18, amino acids 1 to 207 of SEQ ID NO: 19, amino acids 1 to 216 of SEQ ID NO: 20, amino acids 1 to 218 of SEQ ID NO: 21, amino acids 1 to 204 of SEQ ID NO: 22, amino acids 1 to 203 of SEQ ID NO: 23, amino acids 1 to 208 of SEQ ID NO: 24, amino acids 1 to 210 of SEQ ID NO: 25, amino acids 1 to 207 of SEQ ID NO: 26, amino acids 1 to 207 of SEQ ID NO: 27, amino acids 1 to 208 of SEQ ID NO: 28, amino acids 1 to 217 of SEQ ID NO: 29, amino acids 1 to 208 of SEQ ID NO: 30, amino acids 1 to 201 of SEQ ID NO: 31, amino acids 1 to 202 of SEQ ID NO: 32, amino acids 1 to 207 of SEQ ID NO: 33, amino acids 1 to 202 of SEQ ID NO: 34, amino acids 1 to 201 of SEQ ID NO: 35, amino acids 1 to 202 of SEQ ID NO: 36, amino acids 1 to 206 of SEQ ID NO: 37, amino acids 1 to 202 of SEQ ID NO: 38, amino acids 1 to 202 of SEQ ID NO: 39, amino acids 1 to 202 of SEQ ID NO: 40, amino acids 1 to 202 of SEQ ID NO: 41, amino acids 1 to 206 of SEQ ID NO: 42, amino acids 1 to 207 of SEQ ID NO: 43, amino acids 1 to 208 of SEQ ID NO: 44, amino acids 1 to 215 of SEQ ID NO: 45, amino acids 1 to 217 of SEQ ID NO: 46, amino acids 1 to 214 of SEQ ID NO: 47, amino acids 1 to 208 of SEQ ID NO: 48, amino acids 1 to 203 of SEQ ID NO: 49, amino acids 1 to 216 of SEQ ID NO: 50, amino acids 1 to 207 of SEQ ID NO: 51, amino acids 1 to 208 of SEQ ID NO: 52, amino acids 1 to 207 of SEQ ID NO: 53, amino acids 1 to 208 of SEQ ID NO: 54, amino acids 1 to 207 of SEQ ID NO: 55, amino acids 1 to 207 of SEQ ID NO: 56, amino acids 1 to 208 of SEQ ID NO: 57, amino acids 1 to 207 of SEQ ID NO: 58, amino acids 1 to 207 of SEQ ID NO: 59, amino acids 1 to 207 of SEQ ID NO: 60, amino acids 1 to 204 of SEQ ID NO: 61, amino acids 1 to 216 of SEQ ID NO: 62, amino acids 1 to 245 of SEQ ID NO: 63, amino acids 1 to 249 of SEQ ID NO: 64, amino acids 1 to 248 of SEQ ID NO: 65, amino acids 1 to 245 of SEQ ID NO: 66, amino acids 1 to 249 of SEQ ID NO: 67, amino acids 1 to 245 of SEQ ID NO: 68, amino acids 1 to 247 of SEQ ID NO: 69, amino acids 1 to 250 of SEQ ID NO: 70 or amino acids 1 to 240 of SEQ ID NO: 71.

Examples of conservative substitutions are within the groups of basic amino acids (arginine, lysine and histidine), acidic amino acids (glutamic acid and aspartic acid), polar amino acids (glutamine and asparagine), hydrophobic amino acids (leucine, isoleucine and valine), aromatic amino acids (phenylalanine, tryptophan and tyrosine), and small amino acids (glycine, alanine, serine, threonine and methionine). Amino acid substitutions that do not generally alter specific activity are known in the art and are described, for example, by H. Neurath and R. L. Hill, 1979, In, The Proteins, Academic Press, New York. Common substitutions are Ala/Ser, Val/Ile, Asp/Glu, Thr/Ser, Ala/Gly, Ala/Thr, Ser/Asn, Ala/Val, Ser/Gly, Tyr/Phe, Ala/Pro, Lys/Arg, Asp/Asn, Leu/Ile, Leu/Val, Ala/Glu, and Asp/Gly. Other examples of conservative substitutions are G to A; A to G, S; V to I, L, A, T, S; I to V, L, M; L to I, M, V; M to L, I, V; P to A, S, N; F to Y, W, H; Y to F, W, H; W to Y, F, H; R to K, E, D; K to R, E, D; H to Q, N, S; D to N, E, K, R, Q; E to Q, D, K, R, N; S to T, A; T to S, V, A; C to S, T, A; N to D, Q, H, S; Q to E, N, H, K, R.

Essential amino acids in a polypeptide can be identified according to procedures known in the art, such as site-directed mutagenesis or alanine-scanning mutagenesis (Cunningham and Wells, 1989, Science 244: 1081-1085). In the latter technique, single alanine mutations are introduced at every residue in the molecule, and the resultant mutant molecules are tested for muramidase activity to identify amino acid residues that are critical to the activity of the molecule. See also, Hilton et al., 1996, J. Biol. Chem. 271: 4699-4708. The active site of the enzyme or other biological interaction can also be determined by physical analysis of structure, as determined by such techniques as nuclear magnetic resonance, crystallography, electron diffraction, or photoaffinity labelling, in conjunction with mutation of putative contact site amino acids. See, for example, de Vos et al., 1992, Science 255: 306-312; Smith et al., 1992, J. Mol. Biol. 224: 899-904; Wlodaver et al., 1992, FEBS Lett. 309: 59-64. The identity of essential amino acids can also be inferred from an alignment with a related polypeptide.

WO 2013/076253 disclosed that amino acid residues D95 and E97 of SEQ ID NO: 8 of WO 2013/076253 are catalytic residues. PCT/CN2017/075960 discloses the catalytic amino acids of 12 GH25 muramidases. This alignment can be used to determine the position of the catalytic amino acids for the claimed muramidases. In one embodiment, no alteration is made to an amino acid corresponding to E97 and D95 when using SEQ ID NO: 39 for numbering. The catalytic amino acids for the GH24 muramidases can be determined by aligning the sequences with known sequences where the catalytic amino acid(s) have already been determined (see www.uniprot.org).

In one embodiment, the invention relates to an animal feed comprising an animal feed additive, one or more protein sources and one or more energy sources characterised in that the animal feed or the animal feed additive further comprises one or more polypeptides having xylanase activity and one or more fungal polypeptides having muramidase activity, wherein the polypeptide having xylanase activity activity is selected from the group consisting of:
(a) a polypeptide having at least 80%, e.g., at least 85%, at least 90%, at least 95%, or 100% sequence identity to SEQ ID NO: 72;
(b) a polypeptide having at least 80%, e.g., at least 85%, at least 90%, at least 95%, or 100% sequence identity to SEQ ID NO: 73;
(c) a polypeptide having at least 80%, e.g., at least 85%, at least 90%, at least 95%, or 100% sequence identity to SEQ ID NO: 74;
(d) a polypeptide having at least 80%, e.g., at least 85%, at least 90%, at least 95%, or 100% sequence identity to SEQ ID NO: 75;
(e) a polypeptide having at least 80%, e.g., at least 85%, at least 90%, at least 95%, or 100% sequence identity to SEQ ID NO: 76;
(f) a polypeptide having at least 80%, e.g., at least 85%, at least 90%, at least 95%, or 100% sequence identity to SEQ ID NO: 77;
(g) a polypeptide having at least 80%, e.g., at least 85%, at least 90%, at least 95%, or 100% sequence identity to SEQ ID NO: 78;
(h) a polypeptide having at least 80%, e.g., at least 85%, at least 90%, at least 95%, or 100% sequence identity to SEQ ID NO: 79;
(i) a polypeptide having at least 80%, e.g., at least 85%, at least 90%, at least 95%, or 100% sequence identity to SEQ ID NO: 80;
(j) a polypeptide having at least 80%, e.g., at least 85%, at least 90%, at least 95%, or 100% sequence identity to SEQ ID NO: 81;
(k) a polypeptide having at least 80%, e.g., at least 85%, at least 90%, at least 95%, or 100% sequence identity to SEQ ID NO: 82;
(l) a polypeptide having at least 80%, e.g., at least 85%, at least 90%, at least 95%, or 100% sequence identity to SEQ ID NO: 83;
(m) a polypeptide having at least 80%, e.g., at least 85%, at least 90%, at least 95%, or 100% sequence identity to SEQ ID NO: 84;
(n) a polypeptide having at least 80%, e.g., at least 85%, at least 90%, at least 95%, or 100% sequence identity to SEQ ID NO: 85;
(o) a polypeptide having at least 80%, e.g., at least 85%, at least 90%, at least 95%, or 100% sequence identity to SEQ ID NO: 86;
(p) a polypeptide having at least 80%, e.g., at least 85%, at least 90%, at least 95%, or 100% sequence identity to SEQ ID NO: 87;
(q) a polypeptide having at least 80%, e.g., at least 85%, at least 90%, at least 95%, or 100% sequence identity to SEQ ID NO: 88;
(r) a polypeptide having at least 80%, e.g., at least 85%, at least 90%, at least 95%, or 100% sequence identity to SEQ ID NO: 89;
(s) a polypeptide having at least 80%, e.g., at least 85%, at least 90%, at least 95%, or 100% sequence identity to SEQ ID NO: 90;
(t) a polypeptide having at least 80%, e.g., at least 85%, at least 90%, at least 95%, or 100% sequence identity to SEQ ID NO: 91;
(u) a polypeptide having at least 80%, e.g., at least 85%, at least 90%, at least 95%, or 100% sequence identity to SEQ ID NO: 92;
(v) a polypeptide having at least 80%, e.g., at least 85%, at least 90%, at least 95%, or 100% sequence identity to SEQ ID NO: 93;
(w) a polypeptide having at least 80%, e.g., at least 85%, at least 90%, at least 95%, or 100% sequence identity to SEQ ID NO: 94;
(x) a polypeptide having at least 80%, e.g., at least 85%, at least 90%, at least 95%, or 100% sequence identity to SEQ ID NO: 95;
(y) a polypeptide having at least 80%, e.g., at least 85%, at least 90%, at least 95%, or 100% sequence identity to SEQ ID NO: 96;
(z) a polypeptide having at least 80%, e.g., at least 85%, at least 90%, at least 95%, or 100% sequence identity to SEQ ID NO: 97;
(aa) a polypeptide having at least 80%, e.g., at least 85%, at least 90%, at least 95%, or 100% sequence identity to SEQ ID NO: 98;
(ab) a polypeptide having at least 80%, e.g., at least 85%, at least 90%, at least 95%, or 100% sequence identity to SEQ ID NO: 99;
(ac) a polypeptide having at least 80%, e.g., at least 85%, at least 90%, at least 95%, or 100% sequence identity to SEQ ID NO: 100;
(ad) a polypeptide having at least 80%, e.g., at least 85%, at least 90%, at least 95%, or 100% sequence identity to SEQ ID NO: 101;
(ae) a polypeptide having at least 80%, e.g., at least 85%, at least 90%, at least 95%, or 100% sequence identity to SEQ ID NO: 102;
(af) a polypeptide having at least 80%, e.g., at least 85%, at least 90%, at least 95%, or 100% sequence identity to SEQ ID NO: 103;
(ag) a polypeptide having at least 80%, e.g., at least 85%, at least 90%, at least 95%, or 100% sequence identity to SEQ ID NO: 104;
(ah) a polypeptide having at least 80%, e.g., at least 85%, at least 90%, at least 95%, or 100% sequence identity to SEQ ID NO: 105;
(ai) a polypeptide having at least 80%, e.g., at least 85%, at least 90%, at least 95%, or 100% sequence identity to SEQ ID NO: 106;
(aj) a polypeptide having at least 80%, e.g., at least 85%, at least 90%, at least 95%, or 100% sequence identity to SEQ ID NO: 107;
(ak) a polypeptide having at least 80%, e.g., at least 85%, at least 90%, at least 95%, or 100% sequence identity to SEQ ID NO: 108;
(al) a polypeptide having at least 80%, e.g., at least 85%, at least 90%, at least 95%, or 100% sequence identity to SEQ ID NO: 109;
(am) a polypeptide having at least 80%, e.g., at least 85%, at least 90%, at least 95%, or 100% sequence identity to SEQ ID NO: 110;
(an) a polypeptide having at least 80%, e.g., at least 85%, at least 90%, at least 95%, or 100% sequence identity to SEQ ID NO: 111;
(ao) a polypeptide having at least 80%, e.g., at least 85%, at least 90%, at least 95%, or 100% sequence identity to SEQ ID NO: 112;
(ap) a polypeptide having at least 80%, e.g., at least 85%, at least 90%, at least 95%, or 100% sequence identity to SEQ ID NO: 113;
(aq) a polypeptide having at least 80%, e.g., at least 85%, at least 90%, at least 95%, or 100% sequence identity to SEQ ID NO: 114;
(ar) a polypeptide having at least 80%, e.g., at least 85%, at least 90%, at least 95%, or 100% sequence identity to SEQ ID NO: 115;
(as) a polypeptide having at least 80%, e.g., at least 85%, at least 90%, at least 95%, or 100% sequence identity to SEQ ID NO: 116;
(at) a polypeptide having at least 80%, e.g., at least 85%, at least 90%, at least 95%, or 100% sequence identity to SEQ ID NO: 117;
(au) a polypeptide having at least 80%, e.g., at least 85%, at least 90%, at least 95%, or 100% sequence identity to SEQ ID NO: 118;
(av) a polypeptide having at least 80%, e.g., at least 85%, at least 90%, at least 95%, or 100% sequence identity to SEQ ID NO: 119;
(aw) a polypeptide having at least 80%, e.g., at least 85%, at least 90%, at least 95%, or 100% sequence identity to SEQ ID NO: 120;
(ax) a polypeptide having at least 80%, e.g., at least 85%, at least 90%, at least 95%, or 100% sequence identity to SEQ ID NO: 121;
(ay) a polypeptide having at least 80%, e.g., at least 85%, at least 90%, at least 95%, or 100% sequence identity to SEQ ID NO: 122;
(az) a polypeptide having at least 80%, e.g., at least 85%, at least 90%, at least 95%, or 100% sequence identity to SEQ ID NO: 123;
(ba) a polypeptide having at least 80%, e.g., at least 85%, at least 90%, at least 95%, or 100% sequence identity to SEQ ID NO: 124;
(bb) a polypeptide having at least 80%, e.g., at least 85%, at least 90%, at least 95%, or 100% sequence identity to SEQ ID NO: 125;
(bc) a polypeptide having at least 80%, e.g., at least 85%, at least 90%, at least 95%, or 100% sequence identity to SEQ ID NO: 126;
(bd) a variant of SEQ ID NO: 72, SEQ ID NO: 73, SEQ ID NO: 74, SEQ ID NO: 75, SEQ ID NO: 76, SEQ ID NO: 77, SEQ ID NO: 78, SEQ ID NO: 79, SEQ ID NO: 80, SEQ ID NO: 81, SEQ ID NO: 82, SEQ ID NO: 83, SEQ ID NO: 84, SEQ ID NO: 85, SEQ ID NO: 86, SEQ ID NO: 87, SEQ ID NO: 88, SEQ ID NO: 89, SEQ ID NO: 90, SEQ ID NO: 91, SEQ ID NO: 92, SEQ ID NO: 93, SEQ ID NO: 94, SEQ ID NO: 95, SEQ ID NO: 96, SEQ ID NO: 97, SEQ ID NO: 98, SEQ ID NO: 99, SEQ ID NO: 100, SEQ ID NO: 101, SEQ ID NO: 102, SEQ ID NO: 103, SEQ ID NO: 104, SEQ ID NO: 105, SEQ ID NO: 106, SEQ ID NO: 107, SEQ ID NO: 108, SEQ ID NO: 109, SEQ ID NO: 110, SEQ ID NO: 111, SEQ ID NO: 112, SEQ ID NO: 113, SEQ ID NO: 114, SEQ ID NO: 115, SEQ ID NO: 116, SEQ ID NO: 117, SEQ ID NO: 118, SEQ ID NO: 119, SEQ ID NO: 120, SEQ ID NO: 121, SEQ ID NO: 122, SEQ ID NO: 123, SEQ ID NO: 124, SEQ ID NO: 125 or SEQ ID NO: 126 comprising one or more amino acid substitutions (preferably conservative substitutions), and/or one or more amino acid deletions, and/or one or more amino acid insertions or any combination thereof in 1, 2, 3, 4, 5, 6, 7, 8, 9 or 10 positions;
(be) a polypeptide comprising the polypeptide of (a), (b), (c), (d), (e), (f), (g), (h), (i), (j), (k), (l), (m), (n), (o), (p), (q), (r), (s), (t), (u), (v), (w), (x), (y), (z), (aa), (ab), (ac), (ad), (ae), (af), (ag), (ah), (ai), (aj), (ak), (al), (am), (an), (ao), (ap), (aq), (ar), (as), (at), (au), (av), (aw), (ax), (ay), (az), (ba), (bb), (bc) or (bd) and a N-terminal and/or C-terminal extension of between 1 and 10 amino acids; and
(bf) a fragment of the polypeptide of (a), (b), (c), (d), (e), (f), (g), (h), (i), (j), (k), (l), (m), (n), (o), (p), (q), (r), (s), (t), (u), (v), (w), (x), (y), (z), (aa), (ab), (ac), (ad), (ae), (af), (ag), (ah), (ai), (aj), (ak), (al), (am), (an), (ao), (ap), (aq), (ar), (as), (at), (au), (av), (aw), (ax), (ay), (az), (ba), (bb), (bc) or (bd) having xylanase activity and having at least 90% of the length of the mature polypeptide.

In one embodiment, the xylanase comprises or consists of amino acids amino acids 1 to 384 of SEQ ID NO: 72, amino acids 1 to 288 of SEQ ID NO: 73, amino acids 1 to 308 of SEQ ID NO: 74, amino acids 1 to 328 of SEQ ID NO: 75, amino acids 1 to 337 of SEQ ID NO: 76, amino acids 1 to 323 of SEQ ID NO: 77, amino acids 1 to 381 of SEQ ID NO: 78, amino acids 1 to 386 of SEQ ID NO: 79, amino acids 1 to 208 of SEQ ID NO: 80, amino acids 1 to 203 of SEQ ID NO: 81, amino acids 1 to 206 of SEQ ID NO: 82, amino acids 1 to 185 of SEQ ID NO: 83, amino acids 1 to 190 of SEQ ID NO: 84, amino acids 1 to 220 of SEQ ID NO: 85, amino acids 1 to 204 of SEQ ID NO: 86, amino acids 1 to 210 of SEQ ID NO: 87, amino acids 1 to 185 of SEQ ID NO: 88, amino acids 1 to 264 of SEQ ID NO: 89, amino acids 1 to 195 of SEQ ID NO: 90, amino acids 1 to 203 of SEQ ID NO: 91, amino acids 1 to 182 of SEQ ID NO: 92, amino acids 1 to 183 of SEQ ID NO: 93, amino acids 1 to 299 of SEQ ID NO: 94, amino acids 1 to 188 of SEQ ID NO: 95, amino acids 1 to 189 of SEQ ID NO: 96, amino acids 1 to 537 of SEQ ID NO: 97, amino acids 1 to 547 of SEQ ID NO: 98, amino acids 1 to 598 of SEQ ID NO: 99, amino acids 1 to 550 of SEQ ID NO: 100, amino acids 1 to 828 of SEQ ID NO: 101, amino acids 1 to 577 of SEQ ID NO: 102, amino acids 1 to 537 of SEQ ID NO: 103, amino acids 1 to 536 of SEQ ID NO: 104, amino acids 1 to 536 of SEQ ID NO: 105, amino acids 1 to 535 of SEQ ID NO: 106, amino acids 1 to 536 of SEQ ID NO: 107, amino acids 1 to 536 of SEQ ID NO: 108, amino acids 1 to 536 of SEQ ID NO: 109, amino acids 1 to 536 of SEQ ID NO: 110, amino acids 1 to 391 of SEQ ID NO: 111, amino acids 1 to 391 of SEQ ID NO: 112, amino acids 1 to 392 of SEQ ID NO: 113, amino acids 1 to 391 of SEQ ID NO: 114, amino acids 1 to 393 of SEQ ID NO: 115, amino acids 1 to 391 of SEQ ID NO: 116, amino acids 1 to 382 of SEQ ID NO: 117, amino acids 1 to 391 of SEQ ID NO: 118, amino acids 1 to 383 of SEQ ID NO: 119, amino acids 1 to 565 of SEQ ID NO: 120, amino acids 1 to 396 of SEQ ID NO: 121, amino acids 1 to 392 of SEQ ID NO: 122, amino acids 1 to 413 of SEQ ID NO: 123, amino acids 1 to 398 of SEQ ID NO: 124, amino acids 1 to 372 of SEQ ID NO: 125 or amino acids 1 to 557 of SEQ ID NO: 126.

Examples of conservative substitutions are within the groups of basic amino acids (arginine, lysine and histidine), acidic amino acids (glutamic acid and aspartic acid), polar amino acids (glutamine and asparagine), hydrophobic amino acids (leucine, isoleucine and valine), aromatic amino acids (phenylalanine, tryptophan and tyrosine), and small amino acids (glycine, alanine, serine, threonine and methionine). Amino acid substitutions that do not generally alter specific activity are known in the art and are described, for example, by H. Neurath and R. L. Hill, 1979, In, The Proteins, Academic Press, New York. Common substitutions are Ala/Ser, Val/Ile, Asp/Glu, Thr/Ser, Ala/Gly, Ala/Thr, Ser/Asn, Ala/Val, Ser/Gly, Tyr/Phe, Ala/Pro, Lys/Arg, Asp/Asn, Leu/Ile, Leu/Val, Ala/Glu, and Asp/Gly. Other examples of conservative substitutions are G to A; A to G, S; V to I, L, A, T, S; I to V, L, M; L to I, M, V; M to L, I, V; P to A, S, N; F to Y, W, H; Y to F, W, H; W to Y, F, H; R to K, E, D; K to R, E, D; H to Q, N, S; D to N, E, K, R, Q; E to Q, D, K, R, N; S to T, A; T to S, V, A; C to S, T, A; N to D, Q, H, S; Q to E, N, H, K, R.

Essential amino acids in a polypeptide can be identified according to procedures known in the art, such as site-directed mutagenesis or alanine-scanning mutagenesis (Cunningham and Wells, 1989, Science 244: 1081-1085). In the latter technique, single alanine mutations are introduced at every residue in the molecule, and the resultant mutant molecules are tested for muramidase activity to identify amino acid residues that are critical to the activity of the molecule. See also, Hilton et al., 1996, J. Biol. Chem. 271: 4699-4708. The active site of the enzyme or other biological interaction can also be determined by physical analysis of structure, as determined by such techniques as nuclear magnetic resonance, crystallography, electron diffraction, or photoaffinity labelling, in conjunction with mutation of putative contact site amino acids. See, for example, de Vos et al., 1992, Science 255: 306-312; Smith et al., 1992, J. Mol. Biol. 224: 899-904; Wlodaver et al., 1992, FEBS Lett. 309: 59-64. The identity of essential amino acids can also be inferred from an alignment with a related polypeptide.

WO 2017/103159 disclosed that amino acid residues E139 and E229 of Bacillus subtilis sp. 168 are catalytic residues as well as the catalytic amino acids of 10 GH30_8 xylanases. This alignment can be used to determine the position of the catalytic amino acids for the claimed GH30_8 xylanases. In one embodiment, no alteration is made to an amino acid corresponding to E139 and E229 when using SEQ ID NO: 111 for numbering. The catalytic amino acids for the GH5, GH10 and GH11 xylanases can be determined by aligning the sequences with known sequences where the catalytic amino acid(s) have already been determined (see www.uniprot.org).

In a specific embodiment, the invention relates to the muramidase of SEQ ID NO: 1 and the xylanase of SEQ ID NO: 90.

In one embodiment, the BWG is improved by at least 1%, such as by at least 1.0%, at least 1.5% or at least 2.0%. In another embodiment, the BWG is improved by between 1% and 5%, such as between 1.5% and 4%, between 2% and 3%, or any combination of these intervals.

In one embodiment, the FCR is improved by at least 1%, such as by at least 1.0%, at least 1.5% or at least 2.0%. In another embodiment, the FCR is improved by between 1% and 5%, such as between 1.5% and 4%, between 2% and 3%, or any combination of these intervals.

In one embodiment, the EPEF is improved by at least 1%, such as by at least 1.0%, at least 1.5% or at least 2.0%. In another embodiment, the EPEF is improved by between 1% and 5%, such as between 1.5% and 4%, between 2% and 3%, or any combination of these intervals.

In one embodiment, the polypeptide having xylanase activity is dosed at a level of 10 to 1000 mg enzyme protein per kg animal feed, such as 10 to 900 mg, 20 to 800 mg, 30 to 700 mg, 40 to 600 mg, 50 to 500 mg, 60 to 400 mg, 70 to 300 mg, 80 to 200 mg, 90 to 180 mg, 100 to 150 mg enzyme protein per kg animal feed, or any combination of these intervals.

In one embodiment, the polypeptide having muramidase activity is dosed at a level of 100 to 1000 mg enzyme protein per kg animal feed, such as 200 to 900 mg, 300 to 800 mg, 400 to 700 mg or 500 to 600 mg enzyme protein per kg animal feed, or any combination of these intervals.

In one embodiment, the animal is a mono-gastric animal, e.g. pigs or swine (including, but not limited to, piglets, growing pigs, and sows); poultry (including but not limited to poultry, turkey, duck, quail, guinea fowl, goose, pigeon, squab, chicken, broiler, layer, pullet and chick); pet animals (such as cats and dogs); fish (including but not limited to amberjack, arapaima, barb, bass, bluefish, bocachico, bream, bullhead, cachama, carp, catfish, catla, chanos, char, cichlid, cobia, cod, crappie, dorada, drum, eel, goby, goldfish, gourami, grouper, guapote, halibut, java, labeo, lai, loach, mackerel, milkfish, mojarra, mudfish, mullet, paco, pearlspot, pejerrey, perch, pike, pompano, roach, salmon, sampa, sauger, sea bass, seabream, shiner, sleeper, snakehead, snapper, snook, sole, spinefoot, sturgeon, sunfish, sweetfish, tench, terror, tilapia, trout, tuna, turbot, vendace, walleye and whitefish); and crustaceans (including but not limited to shrimps and prawns). In a more preferred embodiment, the animal is selected from the group consisting of swine, poultry, crustaceans and fish. In an even more preferred embodiment, the animal is selected from the group consisting of swine, piglet, growing pig, sow, chicken, broiler, layer, pullet and chick.

In one embodiment, the polypeptide having xylanase activity is formulated as a granule; the polypeptide having muramidase activity is formulated as a granule; or both the polypeptide having xylanase activity is formulated as a granule and the polypeptide having muramidase activity is formulated as a granule. The granule may be formulated as described herein.

In one embodiment, the polypeptide having xylanase activity is formulated as a liquid; the polypeptide having muramidase activity is formulated as a liquid; or both the polypeptide having xylanase activity is formulated as a liquid and the polypeptide having muramidase activity is formulated as a liquid.

In one embodiment, the polypeptide having muramidase activity is dosed between 0.001% to 25% w/w of liquid formulation, preferably 0.01% to 25% w/w, more preferably 0.05% to 20% w/w, more preferably 0.2% to 15% w/w, even more preferably 0.5% to 15% w/w or most preferably 1.0% to 10% w/w polypeptide. In one embodiment, the polypeptide having xylanase activity is dosed between 0.001% to 25% w/w of liquid formulation, preferably 0.01% to 25% w/w, more preferably 0.05% to 20% w/w, more preferably 0.2% to 15% w/w, even more preferably 0.5% to 15% w/w or most preferably 1.0% to 10% w/w polypeptide. In one embodiment, the polypeptide having muramidase activity and the polypeptide having xylanase activity are both dosed between 0.001% to 25% w/w of liquid formulation, preferably 0.01% to 25% w/w, more preferably 0.05% to 20% w/w, more preferably 0.2% to 15% w/w, even more preferably 0.5% to 15% w/w or most preferably 1.0% to 10% w/w polypeptide.

In one embodiment, the liquid formulation further comprises 20%-80% polyol (i.e. total amount of polyol), preferably 25%-75% polyol, more preferably 30%-70% polyol, more preferably 35%-65% polyol or most preferably 40%-60% polyol. In one embodiment, the liquid formulation comprises 20%-80% polyol, preferably 25%-75% polyol, more preferably 30%-70% polyol, more preferably 35%-65% polyol or most preferably 40%-60% polyol wherein the polyol is selected from the group consisting of glycerol, sorbitol, propylene glycol (MPG), ethylene glycol, diethylene glycol, triethylene glycol, 1, 2-propylene glycol or 1, 3-propylene glycol, dipropylene glycol, polyethylene glycol (PEG) having an average molecular weight below about 600 and polypropylene glycol (PPG) having an average molecular weight below about 600. In one embodiment, the liquid formulation comprises 20%-80% polyol (i.e. total amount of polyol), preferably 25%-75% polyol, more preferably 30%-70% polyol, more preferably 35%-65% polyol or most preferably 40%-60% polyol wherein the polyol is selected from the group consisting of glycerol, sorbitol and propylene glycol (MPG).

In one embodiment, the liquid formulation further comprises preservative, preferably selected from the group consisting of sodium sorbate, potassium sorbate, sodium benzoate and potassion benzoate or any combination thereof. In one embodiment, the liquid formulation comprises 0.02% to 1.5% w/w preservative, more preferably 0.05% to 1.0% w/w preservative or most preferably 0.1% to 0.5% w/w preservative. In one embodiment, the liquid formulation comprises 0.001% to 2.0% w/w preservative (i.e. total amount of preservative), preferably 0.02% to 1.5% w/w preservative, more preferably 0.05% to 1.0% w/w preservative or most preferably 0.1% to 0.5% w/w preservative wherein the preservative is selected from the group consisting of sodium sorbate, potassium sorbate, sodium benzoate and potassium benzoate or any combination thereof.

In one embodiment, the liquid formulation comprises one or more formulating agents (such as those described herein), preferably a formulating agent selected from the list consisting of glycerol, ethylene glycol, 1, 2-propylene glycol or 1, 3-propylene glycol, sodium chloride, sodium benzoate, potassium sorbate, sodium sulfate, potassium sulfate, magnesium sulfate, sodium thiosulfate, calcium carbonate, sodium citrate, dextrin, glucose, sucrose, sorbitol, lactose, starch, PVA, acetate and phosphate, preferably selected from the list consisting of 1, 2-propylene glycol, 1, 3-propylene glycol, sodium sulfate, dextrin, cellulose, sodium thiosulfate, kaolin and calcium carbonate.

In one embodiment, the protein source is selected from the group consisting of soybean, wild soybean, beans, lupin, tepary bean, scarlet runner bean, slimjim bean, lima bean, French bean, Broad bean (fava bean), chickpea, lentil, peanut, Spanish peanut, canola, sunflower seed, cotton seed, rapeseed (oilseed rape) or pea or in a processed form such as soybean meal, full fat soy bean meal, soy protein concentrate (SPC), fermented soybean meal (FSBM), sunflower meal, cotton seed meal, rapeseed meal, fish meal, bone meal, feather meal, whey or any combination thereof.

In one embodiment, the energy source is selected from the group consisting of maize, corn, sorghum, barley, wheat, oats, rice, triticale, rye, beet, sugar beet, spinach, potato, cassava, quinoa, cabbage, switchgrass, millet, pearl millet, foxtail millet or in a processed form such as milled corn, milled maize, potato starch, cassava starch, milled sorghum, milled switchgrass, milled millet, milled foxtail millet, milled pearl millet, or any combination thereof.

In one embodiment, the animal feed additive further comprises one or more components selected from the list consisting of one or more additional enzymes; one or more microbes; one or more vitamins; one or more minerals; one or more amino acids; and one or more other feed ingredients, as described herein.

In one embodiment, the animal feed additive further comprises one or more additional enzymes, preferably wherein the enzyme is selected from the group consisting of phytase, galactanase, alpha-galactosidase, beta-galactosidase, protease, phospholipase A1, phospholipase A2, lysophospholipase, phospholipase C, phospholipase D, amylase, arabinofuranosidase, beta-xylosidase, acetyl xylan esterase, feruloyl esterase, cellulase, cellobiohydrolases, beta-glucosidase, pullulanase, mannosidase, mannanase and beta-glucanase or any combination thereof.

In one embodiment, the animal feed additive further comprises one or more microbes, preferably wherein the microbe is selected from the group consisting of Bacillus subtilis, Bacillus licheniformis, Bacillus amyloliquefaciens, Bacillus cereus, Bacillus pumilus, Bacillus polymyxa, Bacillus megaterium, Bacillus coagulans, Bacillus circulans, Bifidobacterium bifidum, Bifidobacterium animalis, Bifidobacterium sp., Carnobacterium sp., Clostridium butyricum, Clostridium sp., Enterococcus faecium, Enterococcus sp., Lactobacillus sp., Lactobacillus acidophilus, Lactobacillus farciminus, Lactobacillus rhamnosus, Lactobacillus reuteri, Lactobacillus salivarius, Lactococcus lactis, Lactococcus sp., Leuconostoc sp., Megasphaera elsdenii, Megasphaera sp., Pediococcus acidilactici, Pediococcus sp., Propionibacterium thoenii, Propionibacterium sp. and Streptococcus sp. or any combination thereof.

In one embodiment, the animal feed additive further comprises one or more vitamins as described herein. In one embodiment, the animal feed additive further comprises one or more minerals as described herein. In one embodiment, the animal feed additive further comprises one or more eubiotics as described herein. In one embodiment, the animal feed additive further comprises one or more prebiotics as described herein. In one embodiment, the animal feed additive further comprises one or more organic acids as described herein. In one embodiment, the animal feed additive further comprises one or more eubiotics as described herein.

### Formulation

The polypeptides having xylanase activity and/or the polypeptides having muramidase activity of the invention may be formulated as a liquid or a solid. For a liquid formulation, the formulating agent may comprise a polyol (such as e.g. glycerol, ethylene glycol or propylene glycol), a salt (such as e.g. sodium chloride, sodium benzoate, potassium sorbate) or a sugar or sugar derivative (such as e.g. dextrin, glucose, sucrose, and sorbitol). Thus in one embodiment, the composition is a liquid composition comprising the polypeptide of the invention and one or more formulating agents selected from the list consisting of glycerol, ethylene glycol, 1,2-propylene glycol, 1,3-propylene glycol, sodium chloride, sodium benzoate, potassium sorbate, dextrin, glucose, sucrose, and sorbitol. The liquid formulation may be sprayed onto the feed after it has been pelleted or may be added to drinking water given to the animals.

For a solid formulation, the formulation may be for example as a granule, spray dried powder or agglomerate (e.g. as disclosed in WO2000/70034). The formulating agent may comprise a salt (organic or inorganic zinc, sodium, potassium or calcium salts such as e.g. such as calcium acetate, calcium benzoate, calcium carbonate, calcium chloride, calcium citrate, calcium sorbate, calcium sulfate, potassium acetate, potassium benzoate, potassium carbonate, potassium chloride, potassium citrate, potassium sorbate, potassium sulfate, sodium acetate, sodium benzoate, sodium carbonate, sodium chloride, sodium citrate, sodium sulfate, zinc acetate, zinc benzoate, zinc carbonate, zinc chloride, zinc citrate, zinc sorbate, zinc sulfate), starch or a sugar or sugar derivative (such as e.g. sucrose, dextrin, glucose, lactose, sorbitol).

In one embodiment, the composition is a solid composition, such as a spray dried composition, comprising the xylanase of the invention and one or more formulating agents selected from the list consisting of sodium chloride, sodium benzoate, potassium sorbate, sodium sulfate, potassium sulfate, magnesium sulfate, sodium thiosulfate, calcium carbonate, sodium citrate, dextrin, glucose, sucrose, sorbitol, lactose, starch and cellulose. In a preferred embodiment, the formulating agent is selected from one or more of the following compounds: sodium sulfate, dextrin, cellulose, sodium thiosulfate, magnesium sulfate and calcium carbonate.

The present invention also relates to enzyme granules/particles comprising the polypeptides having xylanase activity and the polypeptides having muramidase activity of the invention optionally combined with one or more additional enzymes. The granule is composed of a core, and optionally one or more coatings (outer layers) surrounding the core.

Typically the granule/particle size, measured as equivalent spherical diameter (volume based average particle size), of the granule is 20-2000 μm, particularly 50-1500 μm, 100-1500 μm or 250-1200 μm.

The core can be prepared by granulating a blend of the ingredients, e.g., by a method comprising granulation techniques such as crystallization, precipitation, pan-coating, fluid bed coating, fluid bed agglomeration, rotary atomization, extrusion, prilling, spheronization, size reduction methods, drum granulation, and/or high shear granulation.

Methods for preparing the core can be found in Handbook of Powder Technology; Particle size enlargement by C. E. Capes; Volume 1; 1980; Elsevier. Preparation methods include known feed and granule formulation technologies, e.g.:

a) spray dried products, wherein a liquid enzyme-containing solution is atomized in a spray drying tower to form small droplets which during their way down the drying tower dry to form an enzyme-containing particulate material;

b) layered products, wherein the enzyme is coated as a layer around a pre-formed inert core particle, wherein an enzyme-containing solution is atomized, typically in a fluid bed apparatus wherein the pre-formed core particles are fluidized, and the enzyme-containing solution adheres to the core particles and dries up to leave a layer of dry enzyme on the surface of the core particle. Particles of a desired size can be obtained this way if a useful core particle of the desired size can be found. This type of product is described in, e.g., WO 97/23606;

c) absorbed core particles, wherein rather than coating the enzyme as a layer around the core, the enzyme is absorbed onto and/or into the surface of the core. Such a process is described in WO 97/39116.

d) extrusion or pelletized products, wherein an enzyme-containing paste is pressed to pellets or under pressure is extruded through a small opening and cut into particles which are subsequently dried. Such particles usually have a considerable size because of the material in which the extrusion opening is made (usually a plate with bore holes) sets a limit on the allowable pressure drop over the extrusion opening. Also, very high extrusion pressures when using a small opening increase heat generation in the enzyme paste, which is harmful to the enzyme;

e) prilled products, wherein an enzyme-containing powder is suspended in molten wax and the suspension is sprayed, e.g., through a rotating disk atomiser, into a cooling chamber where the droplets quickly solidify (Michael S. Showell (editor); Powdered detergents; Surfactant Science Series; 1998; vol. 71; page 140-142; Marcel Dekker). The product obtained is one wherein the enzyme is uniformly distributed throughout an inert material instead of being concentrated on its surface. Also U.S. Pat. Nos. 4,016,040 and 4,713,245 are documents relating to this technique;

f) mixer granulation products, wherein a liquid is added to a dry powder composition of, e.g., conventional granulating components, the enzyme being introduced either via the liquid or the powder or both. The liquid and the powder are mixed and as the moisture of the liquid is absorbed in the dry powder, the components of the dry powder will start to adhere and agglomerate and particles will build up, forming granulates comprising the enzyme. Such a process is described in U.S. Pat. No. 4,106,991 and related documents EP 170360, EP 304332, EP 304331, WO 90/09440 and WO 90/09428. In a particular product of this process wherein various high-shear mixers can be used as granulators, granulates consisting of enzyme as enzyme, fillers and binders etc. are mixed with cellulose fibres to reinforce the particles to give the so-called T-granulate. Reinforced particles, being more robust, release less enzymatic dust.

g) size reduction, wherein the cores are produced by milling or crushing of larger particles, pellets, tablets, briquettes etc. containing the enzyme. The wanted core particle fraction is obtained by sieving the milled or crushed product. Over and undersized particles can be recycled. Size reduction is described in (Martin Rhodes (editor); Principles of Powder Technology; 1990; Chapter 10; John Wiley & Sons);

h) fluid bed granulation, which involves suspending particulates in an air stream and spraying a liquid onto the fluidized particles via nozzles. Particles hit by spray droplets get wetted and become tacky. The tacky particles collide with other particles and adhere to them and form a granule;

i) the cores may be subjected to drying, such as in a fluid bed drier. Other known methods for drying granules in the feed or detergent industry can be used by the skilled person. The drying preferably takes place at a product temperature of from 25 to 90° C. For some enzymes it is important the cores comprising the enzyme contain a low amount of water before coating. If water sensitive enzymes are coated before excessive water is removed, it will be trapped within the core and it may affect the activity of the enzyme negatively. After drying, the cores preferably contain 0.1-10% w/w water.

The core may include additional materials such as fillers, fibre materials (cellulose or synthetic fibres), stabilizing agents, solubilizing agents, suspension agents, viscosity regulating agents, light spheres, plasticizers, salts, lubricants and fragrances.

The core may include a binder, such as synthetic polymer, wax, fat, or carbohydrate.

The core may include a salt of a multivalent cation, a reducing agent, an antioxidant, a peroxide decomposing catalyst and/or an acidic buffer component, typically as a homogenous blend.

In one embodiment, the core comprises a material selected from the group consisting of salts (such as calcium acetate, calcium benzoate, calcium carbonate, calcium chloride, calcium citrate, calcium sorbate, calcium sulfate, potassium acetate, potassium benzoate, potassium carbonate, potassium chloride, potassium citrate, potassium sorbate, potassium sulfate, sodium acetate, sodium benzoate, sodium carbonate, sodium chloride, sodium citrate, sodium sulfate, zinc acetate, zinc benzoate, zinc carbonate, zinc chloride, zinc citrate, zinc sorbate, zinc sulfate), starch or a sugar or sugar derivative (such as e.g. sucrose, dextrin, glucose, lactose, sorbitol), sugar or sugar derivative (such as e.g. sucrose, dextrin, glucose, lactose, sorbitol), small organic molecules, starch, flour, cellulose and minerals and clay minerals (also known as hydrous aluminium phyllosilicates). In one embodiment, the core comprises a clay mineral such as kaolinite or kaolin.

The core may include an inert particle with the enzyme absorbed into it, or applied onto the surface, e.g., by fluid bed coating.

The core may have a diameter of 20-2000 μm, particularly 50-1500 μm, 100-1500 μm or 250-1200 μm.

The core may be surrounded by at least one coating, e.g., to improve the storage stability, to reduce dust formation during handling, or for coloring the granule. The optional coating(s) may include a salt and/or wax and/or flour coating, or other suitable coating materials.

The coating may be applied in an amount of at least 0.1% by weight of the core, e.g., at least 0.5%, 1% or 5%. The amount may be at most 100%, 70%, 50%, 40% or 30%.

The coating is preferably at least 0.1 μm thick, particularly at least 0.5 μm, at least 1 μm or at least 5 μm. In some embodiments the thickness of the coating is below 100 μm, such as below 60 μm, or below 40 μm.

The coating should encapsulate the core unit by forming a substantially continuous layer. A substantially continuous layer is to be understood as a coating having few or no holes, so that the core unit is encapsulated or enclosed with few or no uncoated areas. The layer or coating should in particular be homogeneous in thickness.

The coating can further contain other materials as known in the art, e.g., fillers, antisticking agents, pigments, dyes, plasticizers and/or binders, such as titanium dioxide, kaolin, calcium carbonate or talc.

A salt coating may comprise at least 60% by weight of a salt, e.g., at least 65%, at least 70%, at least 75%, at least 80%, at least 85%, at least 90%, at least 95% or at least 99% by weight.

The salt may be added from a salt solution where the salt is completely dissolved or from a salt suspension wherein the fine particles are less than 50 μm, such as less than 10 μm or less than 5 μm.

The salt coating may comprise a single salt or a mixture of two or more salts. The salt may be water soluble, in particular having a solubility at least 0.1 g in 100 g of water at 20° C., preferably at least 0.5 g per 100 g water, e.g., at least 1 g per 100 g water, e.g., at least 5 g per 100 g water.

The salt may be an inorganic salt, e.g., salts of sulfate, sulfite, phosphate, phosphonate, nitrate, chloride or carbonate or salts of simple organic acids (less than 10 carbon atoms, e.g., 6 or less carbon atoms) such as citrate, malonate or acetate. Examples of cations in these salts are alkali or earth alkali metal ions, the ammonium ion or metal ions of the first transition series, such as sodium, potassium, magnesium, calcium, zinc or aluminium. Examples of anions include chloride, bromide, iodide, sulfate, sulfite, bisulfite, thiosulfate, phosphate, monobasic phosphate, dibasic phosphate, hypophosphite, dihydrogen pyrophosphate, tetraborate, borate, carbonate, bicarbonate, metasilicate, citrate, malate, maleate, malonate, succinate, sorbate, lactate, formate, acetate, butyrate, propionate, benzoate, tartrate, ascorbate or gluconate. In particular alkali- or earth alkali metal salts of sulfate, sulfite, phosphate, phosphonate, nitrate, chloride or carbonate or salts of simple organic acids such as citrate, malonate or acetate may be used.

The salt in the coating may have a constant humidity at 20° C. above 60%, particularly above 70%, above 80% or above 85%, or it may be another hydrate form of such a salt (e.g., anhydrate). The salt coating may be as described in WO1997/05245, WO1998/54980, WO1998/55599, WO2000/70034, WO2006/034710, WO2008/017661, WO2008/017659, WO2000/020569, WO2001/004279, WO1997/05245, WO2000/01793, WO2003/059086, WO2003/059087, WO2007/031483, WO2007/031485, WO2007/044968, WO2013/192043, WO2014/014647 and WO2015/197719 or polymer coating such as described in WO 2001/00042.

Specific examples of suitable salts are NaCl (CH20° C.=76%), Na2CO3 (CH20° C.=92%), NaNO3 (CH20° C.=73%), Na2HPO4 (CH20° C.=95%), Na3PO4 (CH25° C.=92%), NH4Cl (CH20° C.=79.5%), (NH4)2HPO4 (CH20° C.=93.0%), NH4H2PO4 (CH20° C.=93.1%), (NH4)2SO4 (CH20° C.=81.1%), KCl (CH20° C.=85%), K2HPO4 (CH20° C.=92%), KH2PO4 (CH20° C.=96.5%), KNO3 (CH20° C.=93.5%), Na2SO4 (CH20° C.=93%), K2504 (CH20° C.=98%), KHSO4 (CH20° C.=86%), MgSO4 (CH20° C.=90%), ZnSO4 (CH20° C.=90%) and sodium citrate (CH25° C.=86%). Other examples include NaH2PO4, (NH4)H2PO4, CuSO4, Mg(NO3)2, magnesium acetate, calcium acetate, calcium benzoate, calcium carbonate, calcium chloride, calcium citrate, calcium sorbate, calcium sulfate, potassium acetate, potassium benzoate, potassium carbonate, potassium chloride, potassium citrate, potassium sorbate, sodium acetate, sodium benzoate, sodium citrate, sodium sulfate, zinc acetate, zinc benzoate, zinc carbonate, zinc chloride, zinc citrate and zinc sorbate.

The salt may be in anhydrous form, or it may be a hydrated salt, i.e. a crystalline salt hydrate with bound water(s) of crystallization, such as described in WO 99/32595. Specific examples include anhydrous sodium sulfate (Na2SO4), anhydrous magnesium sulfate (MgSO4), magnesium sulfate heptahydrate (MgSO4.7H2O), zinc sulfate heptahydrate (ZnSO4.7H2O), sodium phosphate dibasic heptahydrate (Na2HPO4.7H2O), magnesium nitrate hexahydrate (Mg(NO3)2(6H2O)), sodium citrate dihydrate and magnesium acetate tetrahydrate.

Preferably the salt is applied as a solution of the salt, e.g., using a fluid bed.

A wax coating may comprise at least 60% by weight of a wax, e.g., at least 65%, at least 70%, at least 75%, at least 80%, at least 85%, at least 90%, at least 95% or at least 99% by weight.

Specific examples of waxes are polyethylene glycols; polypropylenes; Carnauba wax; Candelilla wax; bees wax; hydrogenated plant oil or animal tallow such as polyethylene glycol (PEG), methyl hydroxy-propyl cellulose (MHPC), polyvinyl alcohol (PVA), hydrogenated ox tallow, hydrogenated palm oil, hydrogenated cotton seeds and/or hydrogenated soy bean oil; fatty acid alcohols; mono-glycerides and/or di-glycerides, such as glyceryl stearate, wherein stearate is a mixture of stearic and palmitic acid; micro-crystalline wax; paraffin's; and fatty acids, such as hydrogenated linear long chained fatty acids and derivatives thereof. A preferred wax is palm oil or hydrogenated palm oil.

The granule may comprise a core comprising the xylanase of the invention, one or more salt coatings and one or more wax coatings. Examples of enzyme granules with multiple coatings are shown in WO1993/07263, WO1997/23606 and WO2016/149636.

Non-dusting granulates may be produced, e.g., as disclosed in U.S. Pat. Nos. 4,106,991 and 4,661,452 and may optionally be coated by methods known in the art. The coating materials can be waxy coating materials and film-forming coating materials. Examples of waxy coating materials are poly(ethylene oxide) products (polyethyleneglycol, PEG) with mean molar weights of 1000 to 20000; ethoxylated nonylphenols having from 16 to 50 ethylene oxide units; ethoxylated fatty alcohols in which the alcohol contains from 12 to 20 carbon atoms and in which there are 15 to 80 ethylene oxide units; fatty alcohols; fatty acids; and mono- and di- and triglycerides of fatty acids. Examples of film-forming coating materials suitable for application by fluid bed techniques are given in GB 1483591.

The granulate may further comprise one or more additional enzymes. Each enzyme will then be present in more granules securing a more uniform distribution of the enzymes, and also reduces the physical segregation of different enzymes due to different particle sizes. Methods for producing multi-enzyme co-granulates is disclosed in the ip.com disclosure IPCOM000200739D.

Another example of formulation of enzymes by the use of co-granulates is disclosed in WO 2013/188331.

The present invention also relates to protected enzymes prepared according to the method disclosed in EP 238,216.

Thus, in a further aspect, the present invention provides a granule, which comprises:

(a) a core comprising the polypeptides having xylanase activity and the polypeptides having muramidase activity according to the invention, and

(b) a coating consisting of one or more layer(s) surrounding the core.

In one embodiment, the coating comprises a salt coating as described herein. In one embodiment, the coating comprises a wax coating as described herein. In one embodiment, the coating comprises a salt coating followed by a wax coating as described herein. In one embodiment, the polypeptide having xylanase activity and the polypeptide having muramidase activity are co-granulated.

Co-Granules Comprising Polypeptides Having Muramidase Activity and Polypeptides Having xylanase activity

In a second aspect, the invention relates to co-granule comprising one or more polypeptides having xylanase activity and one or more fungal polypeptides having muramidase activity.

In one embodiment, the muramidase is a GH24 muramidase, preferably a fungal GH24 muramidase, preferably obtained or obtainable from the phylum Ascomycota, more preferably from the class Eurotiomycetes. In one embodiment, the muramidase is a GH25 muramidase, preferably a fungal GH25 muramidase, preferably obtained or obtainable from the phylum Ascomycota, more preferably from the class Eurotiomycetes. In one embodiment, the xylanase is a GH10 xylanase. In one embodiment, the xylanase is a GH11 xylanase. In one embodiment, the xylanase is a GH5 xylanase, preferably a GH5 subfamily 21 or 35 xylanase (herein written GH5_21 and GH5_35 respectively). In one embodiment, the xylanase is a GH30 xylanase, preferably a GH30 subfamily 8 xylanase (herein written GH30_8).

In one embodiment, the muramidase is a GH24 muramidase, preferably a fungal GH24 muramidase, and the xylanase is a GH10 xylanase. In one embodiment, the muramidase is a GH24 muramidase, preferably a fungal GH24 muramidase, and the xylanase is a GH11 xylanase. In one embodiment, the muramidase is a GH24 muramidase, preferably a fungal GH24 muramidase, and the xylanase is a GH5 xylanase. In one embodiment, the muramidase is a GH24 muramidase, preferably a fungal GH24 muramidase, and the xylanase is a GH5_21 xylanase. In one embodiment, the muramidase is a GH24 muramidase, preferably a fungal GH24 muramidase, and the xylanase is a GH5_35 xylanase. In one embodiment, the muramidase is a GH24 muramidase, preferably a fungal GH24 muramidase, and the xylanase is a GH30 xylanase. In one embodiment, the muramidase is a GH24 muramidase, preferably a fungal GH24 muramidase, and the xylanase is a GH30_8 xylanase. In one embodiment, the muramidase is obtained or obtainable from the phylum Ascomycota, more preferably from the class Eurotiomycetes.

In one embodiment, the muramidase is a GH25 muramidase, preferably a fungal GH25 muramidase, and the xylanase is a GH10 xylanase. In one embodiment, the muramidase is a GH25 muramidase, preferably a fungal GH25 muramidase, and the xylanase is a GH11 xylanase. In one embodiment, the muramidase is a GH25 muramidase, preferably a fungal GH25 muramidase, and the xylanase is a GH5 xylanase. In one embodiment, the muramidase is a GH25 muramidase, preferably a fungal GH25 muramidase, and the xylanase is a GH5_21 xylanase. In one embodiment, the muramidase is a GH25 muramidase, preferably a fungal GH25 muramidase, and the xylanase is a GH5_35 xylanase. In one embodiment, the muramidase is a GH25 muramidase, preferably a fungal GH25 muramidase, and the xylanase is a GH30 xylanase. In one embodiment, the muramidase is a GH25 muramidase, preferably a fungal GH25 muramidase, and the xylanase is a GH30_8 xylanase. In one embodiment, the muramidase is obtained or obtainable from the phylum Ascomycota, more preferably from the class Eurotiomycetes.

In one embodiment, the muramidase is a fungal GH24 muramidase that degrades cell wall debris from Lactobacillus johnsonii and the xylanase is a GH10 xylanase. In one embodiment, the muramidase is a fungal GH24 muramidase that degrades cell wall debris from Lactobacillus johnsonii and the xylanase is a GH11 xylanase. In one embodiment, the muramidase is a fungal GH24 muramidase that degrades cell wall debris from Lactobacillus johnsonii and the xylanase is a GH5 xylanase. In one embodiment, the muramidase is a fungal GH24 muramidase that degrades cell wall debris from Lactobacillus johnsonii and the xylanase is a GH5_21 xylanase. In one embodiment, the muramidase is a fungal GH24 muramidase that degrades cell wall debris from Lactobacillus johnsonii and the xylanase is a GH5_35 xylanase. In one embodiment, the muramidase is a fungal GH24 muramidase that degrades cell wall debris from Lactobacillus johnsonii and the xylanase is a GH30 xylanase. In one embodiment, the muramidase is a fungal GH24 muramidase that degrades cell wall debris from Lactobacillus johnsonii and the xylanase is a GH30_8 xylanase. In one embodiment, the muramidase is obtained or obtainable from the phylum Ascomycota, more preferably from the class Eurotiomycetes.

In one embodiment, the muramidase is a fungal GH25 muramidase that degrades cell wall debris from Lactobacillus johnsonii and the xylanase is a GH10 xylanase. In one embodiment, the muramidase is a fungal GH25 muramidase that degrades cell wall debris from Lactobacillus johnsonii and the xylanase is a GH11 xylanase. In one embodiment, the muramidase is a fungal GH25 muramidase that degrades cell wall debris from Lactobacillus johnsonii and the xylanase is a GH5 xylanase. In one embodiment, the muramidase is a fungal GH25 muramidase that degrades cell wall debris from Lactobacillus johnsonii and the xylanase is a GH5_21 xylanase. In one embodiment, the muramidase is a fungal GH25 muramidase that degrades cell wall debris from Lactobacillus johnsonii and the xylanase is a GH5_35 xylanase. In one embodiment, the muramidase is a fungal GH25 muramidase that degrades cell wall debris from Lactobacillus johnsonii and the xylanase is a GH30 xylanase. In one embodiment, the muramidase is a fungal GH25 muramidase that degrades cell wall debris from Lactobacillus johnsonii and the xylanase is a GH30_8 xylanase. In one embodiment, the muramidase is obtained or obtainable from the phylum Ascomycota, more preferably from the class Eurotiomycetes.

In one embodiment, the muramidase is selected from the list as described in the first aspect. In one embodiment, the xylanase is selected from the list as described in the first aspect.

In one embodiment, the co-granule further comprises one or more components selected from the list consisting of one or more carriers; one or more additional enzymes; one or more microbes; one or more vitamins; one or more minerals, as described herein.

In one embodiment, the co-granule comprises one or more carriers, preferably wherein the carrier is selected from the group consisting of water, glycerol, ethylene glycol, 1, 2-propylene glycol or 1, 3-propylene glycol, sodium chloride, sodium benzoate, potassium sorbate, sodium sulfate, potassium sulfate, magnesium sulfate, sodium thiosulfate, calcium carbonate, sodium citrate, dextrin, maltodextrin, glucose, sucrose, sorbitol, lactose, wheat flour, wheat bran, corn gluten meal, starch, kaolin and cellulose or any combination thereof.

In one embodiment, the co-granule comprises one or more additional enzymes, preferably wherein the enzyme is selected from the group consisting of phytase, galactanase, alpha-galactosidase, beta-galactosidase, protease, phospholipase A1, phospholipase A2, lysophospholipase, phospholipase C, phospholipase D, amylase, arabinofuranosidase, beta-xylosidase, acetyl xylan esterase, feruloyl esterase, cellulase, cellobiohydrolases, beta-glucosidase, pullulanase, mannosidase, mannanase and beta-glucanase or any combination thereof.

In one embodiment, the co-granule comprises one or more microbes, preferably wherein the microbe is selected from the group consisting of Bacillus subtilis, Bacillus licheniformis, Bacillus amyloliquefaciens, Bacillus cereus, Bacillus pumilus, Bacillus polymyxa, Bacillus megaterium, Bacillus coagulans, Bacillus circulans, Bifidobacterium bifidum, Bifidobacterium animalis, Bifidobacterium sp., Carnobacterium sp., Clostridium butyricum, Clostridium sp., Enterococcus faecium, Enterococcus sp., Lactobacillus sp., Lactobacillus acidophilus, Lactobacillus farciminus, Lactobacillus rhamnosus, Lactobacillus reuteri, Lactobacillus salivarius, Lactococcus lactis, Lactococcus sp., Leuconostoc sp., Megasphaera elsdenii, Megasphaera sp., Pediococsus acidilactici, Pediococcus sp., Propionibacterium thoenii, Propionibacterium sp. and Streptococcus sp. or any combination thereof.

### Liquid Formulations Comprising Polypeptides Having Muramidase Activity and Polypeptides Having Xylanase Activity

In a third aspect, the invention relates to a liquid formulation comprising one or more polypeptides having xylanase activity, one or more fungal polypeptides having muramidase activity and one or more polyols.

In one embodiment, the muramidase is a GH24 muramidase, preferably a fungal GH24 muramidase, preferably obtained or obtainable from the phylum Ascomycota, more preferably from the class Eurotiomycetes. In one embodiment, the muramidase is a GH25 muramidase, preferably a fungal GH25 muramidase, preferably obtained or obtainable from the phylum Ascomycota, more preferably from the class Eurotiomycetes. In one embodiment, the xylanase is a GH10 xylanase. In one embodiment, the xylanase is a GH11 xylanase. In one embodiment, the xylanase is a GH5 xylanase, preferably a GH5 subfamily 21 or 35 xylanase (herein written GH5_21 and GH5_35 respectively). In one embodiment, the xylanase is a GH30 xylanase, preferably a GH30 subfamily 8 xylanase (herein written GH30_8).

In one embodiment, the muramidase is a GH24 muramidase, preferably a fungal GH24 muramidase, and the xylanase is a GH10 xylanase. In one embodiment, the muramidase is a GH24 muramidase, preferably a fungal GH24 muramidase, and the xylanase is a GH11 xylanase. In one embodiment, the muramidase is a GH24 muramidase, preferably a fungal GH24 muramidase, and the xylanase is a GH5 xylanase. In one embodiment, the muramidase is a GH24 muramidase, preferably a fungal GH24 muramidase, and the xylanase is a GH5_21 xylanase. In one embodiment, the muramidase is a GH24 muramidase, preferably a fungal GH24 muramidase, and the xylanase is a GH5_35 xylanase. In one embodiment, the muramidase is a GH24 muramidase, preferably a fungal GH24 muramidase, and the xylanase is a GH30 xylanase. In one embodiment, the muramidase is a GH24 muramidase, preferably a fungal GH24 muramidase, and the xylanase is a GH30_8 xylanase. In one embodiment, the muramidase is obtained or obtainable from the phylum Ascomycota, more preferably from the class Eurotiomycetes.

In one embodiment, the muramidase is a GH25 muramidase, preferably a fungal GH25 muramidase, and the xylanase is a GH10 xylanase. In one embodiment, the muramidase is a GH25 muramidase, preferably a fungal GH25 muramidase, and the xylanase is a GH11 xylanase. In one embodiment, the muramidase is a GH25 muramidase, preferably a fungal GH25 muramidase, and the xylanase is a GH5 xylanase. In one embodiment, the muramidase is a GH25 muramidase, preferably a fungal GH25 muramidase, and the xylanase is a GH5_21 xylanase. In one embodiment, the muramidase is a GH25 muramidase, preferably a fungal GH25 muramidase, and the xylanase is a GH5_35 xylanase. In one embodiment, the muramidase is a GH25 muramidase, preferably a fungal GH25 muramidase, and the xylanase is a GH30 xylanase. In one embodiment, the muramidase is a GH25 muramidase, preferably a fungal GH25 muramidase, and the xylanase is a GH30_8 xylanase. In one embodiment, the muramidase is obtained or obtainable from the phylum Ascomycota, more preferably from the class Eurotiomycetes.

In one embodiment, the muramidase is a fungal GH24 muramidase that degrades cell wall debris from Lactobacillus johnsonii and the xylanase is a GH10 xylanase. In one embodiment, the muramidase is a fungal GH24 muramidase that degrades cell wall debris from Lactobacillus johnsonii and the xylanase is a GH11 xylanase. In one embodiment, the muramidase is a fungal GH24 muramidase that degrades cell wall debris from Lactobacillus johnsonii and the xylanase is a GH5 xylanase. In one embodiment, the muramidase is a fungal GH24 muramidase that degrades cell wall debris from Lactobacillus johnsonii and the xylanase is a GH5_21 xylanase. In one embodiment, the muramidase is a fungal GH24 muramidase that degrades cell wall debris from Lactobacillus johnsonii and the xylanase is a GH5_35 xylanase. In one embodiment, the muramidase is a fungal GH24 muramidase that degrades cell wall debris from Lactobacillus johnsonii and the xylanase is a GH30 xylanase. In one embodiment, the muramidase is a fungal GH24 muramidase that degrades cell wall debris from Lactobacillus johnsonii and the xylanase is a GH30_8 xylanase. In one embodiment, the muramidase is obtained or obtainable from the phylum Ascomycota, more preferably from the class Eurotiomycetes.

In one embodiment, the muramidase is a fungal GH25 muramidase that degrades cell wall debris from Lactobacillus johnsonii and the xylanase is a GH10 xylanase. In one embodiment, the muramidase is a fungal GH25 muramidase that degrades cell wall debris from Lactobacillus johnsonii and the xylanase is a GH11 xylanase. In one embodiment, the muramidase is a fungal GH25 muramidase that degrades cell wall debris from Lactobacillus johnsonii and the xylanase is a GH5 xylanase. In one embodiment, the muramidase is a fungal GH25 muramidase that degrades cell wall debris from Lactobacillus johnsonii and the xylanase is a GH5_21 xylanase. In one embodiment, the muramidase is a fungal GH25 muramidase that degrades cell wall debris from Lactobacillus johnsonii and the xylanase is a GH5_35 xylanase. In one embodiment, the muramidase is a fungal GH25 muramidase that degrades cell wall debris from Lactobacillus johnsonii and the xylanase is a GH30 xylanase. In one embodiment, the muramidase is a fungal GH25 muramidase that degrades cell wall debris from Lactobacillus johnsonii and the xylanase is a GH30_8 xylanase. In one embodiment, the muramidase is obtained or obtainable from the phylum Ascomycota, more preferably from the class Eurotiomycetes.

In one embodiment, the muramidase is selected from the list as described in the first aspect. In one embodiment, the xylanase is selected from the list as described in the first aspect.

In one embodiment, the polypeptide having muramidase activity is dosed between 0.001% to 25% w/w of liquid formulation, preferably 0.01% to 25% w/w, more preferably 0.05% to 20% w/w, more preferably 0.2% to 15% w/w, even more preferably 0.5% to 15% w/w or most preferably 1.0% to 10% w/w polypeptide. In one embodiment, the polypeptide having xylanase activity is dosed between 0.001% to 25% w/w of liquid formulation, preferably 0.01% to 25% w/w, more preferably 0.05% to 20% w/w, more preferably 0.2% to 15% w/w, even more preferably 0.5% to 15% w/w or most preferably 1.0% to 10% w/w polypeptide. In one embodiment, the polypeptide having muramidase activity and the polypeptide having xylanase activity are both dosed between 0.001% to 25% w/w of liquid formulation, preferably 0.01% to 25% w/w, more preferably 0.05% to 20% w/w, more preferably 0.2% to 15% w/w, even more preferably 0.5% to 15% w/w or most preferably 1.0% to 10% w/w polypeptide.

In one embodiment, the liquid formulation comprises 20%-80% polyol (i.e. total amount of polyol), preferably 25%-75% polyol, more preferably 30%-70% polyol, more preferably 35%-65% polyol or most preferably 40%-60% polyol. In one embodiment, the liquid formulation comprises 20%-80% polyol, preferably 25%-75% polyol, more preferably 30%-70% polyol, more preferably 35%-65% polyol or most preferably 40%-60% polyol wherein the polyol is selected from the group consisting of glycerol, sorbitol, propylene glycol (MPG), ethylene glycol, diethylene glycol, triethylene glycol, 1, 2-propylene glycol or 1, 3-propylene glycol, dipropylene glycol, polyethylene glycol (PEG) having an average molecular weight below about 600 and polypropylene glycol (PPG) having an average molecular weight below about 600. In one embodiment, the liquid formulation comprises 20%-80% polyol (i.e. total amount of polyol), preferably 25%-75% polyol, more preferably 30%-70% polyol, more preferably 35%-65% polyol or most preferably 40%-60% polyol wherein the polyol is selected from the group consisting of glycerol, sorbitol and propylene glycol (MPG).

In one embodiment, the liquid formulation further comprises preservative, preferably selected from the group consisting of sodium sorbate, potassium sorbate, sodium benzoate and potassion benzoate or any combination thereof. In one embodiment, the liquid formulation comprises 0.02% to 1.5% w/w preservative, more preferably 0.05% to 1.0% w/w preservative or most preferably 0.1% to 0.5% w/w preservative. In one embodiment, the liquid formulation comprises 0.001% to 2.0% w/w preservative (i.e. total amount of preservative), preferably 0.02% to 1.5% w/w preservative, more preferably 0.05% to 1.0% w/w preservative or most preferably 0.1% to 0.5% w/w preservative wherein the preservative is selected from the group consisting of sodium sorbate, potassium sorbate, sodium benzoate and potassium benzoate or any combination thereof.

In one embodiment, the liquid formulation comprises one or more formulating agents (such as those described herein), preferably a formulating agent selected from the list consisting of glycerol, ethylene glycol, 1, 2-propylene glycol or 1, 3-propylene glycol, sodium chloride, sodium benzoate, potassium sorbate, sodium sulfate, potassium sulfate, magnesium sulfate, sodium thiosulfate, calcium carbonate, sodium citrate, dextrin, glucose, sucrose, sorbitol, lactose, starch, PVA, acetate and phosphate, preferably selected from the list consisting of 1, 2-propylene glycol, 1, 3-propylene glycol, sodium sulfate, dextrin, cellulose, sodium thiosulfate, kaolin and calcium carbonate.

In one embodiment, the liquid formulation further comprises one or more components selected from the list consisting of one or more additional enzymes; one or more microbes; one or more vitamins; one or more minerals, as described herein.

In one embodiment, the liquid formulation comprises one or more additional enzymes, preferably wherein the enzyme is selected from the group consisting of phytase, galactanase, alpha-galactosidase, beta-galactosidase, protease, phospholipase A1, phospholipase A2, lysophospholipase, phospholipase C, phospholipase D, amylase, arabinofuranosidase, beta-xylosidase, acetyl xylan esterase, feruloyl esterase, cellulase, cellobiohydrolases, beta-glucosidase, pullulanase, mannosidase, mannanase and beta-glucanase or any combination thereof.

In one embodiment, the liquid formulation comprises one or more microbes, preferably wherein the microbe is selected from the group consisting of Bacillus subtilis, Bacillus licheniformis, Bacillus amyloliquefaciens, Bacillus cereus, Bacillus pumilus, Bacillus polymyxa, Bacillus megaterium, Bacillus coagulans, Bacillus circulans, Bifidobacterium bifidum, Bifidobacterium animalis, Bifidobacterium sp., Carnobacterium sp., Clostridium butyricum, Clostridium sp., Enterococcus faecium, Enterococcus sp., Lactobacillus sp., Lactobacillus acidophilus, Lactobacillus farciminus, Lactobacillus rhamnosus, Lactobacillus reuteri, Lactobacillus salivarius, Lactococcus lactis, Lactococcus sp., Leuconostoc sp., Megasphaera elsdenii, Megasphaera sp., Pediococcus acidilactici, Pediococcus sp., Propionibacterium thoenii, Propionibacterium sp. and Streptococcus sp. or any combination thereof.

### Packaging

In a fourth aspect, the invention relates to packaging comprising granules comprising a polypeptide having xylanase activity and granules comprising a fungal polypeptide having muramidase activity, as described herein in the first and second aspects. In one embodiment, the packaging is a bottle, a can, a drum or a big bag.

### Animal Feed

Animal feed compositions or diets have a relatively high content of protein. Poultry and pig diets can be characterised as indicated in Table B of WO 01/58275, columns 2-3. Fish diets can be characterised as indicated in column 4 of this Table B. Furthermore such fish diets usually have a crude fat content of 200-310 g/kg.

An animal feed composition according to the invention has a crude protein content of 50-800 g/kg, and furthermore comprises one or more polypeptides having xylanase activity and one or more polypeptides having muramidase activity as described herein.

Furthermore, or in the alternative (to the crude protein content indicated above), the animal feed composition of the invention has a content of metabolisable energy of 10-30 MJ/kg; and/or a content of calcium of 0.1-200 g/kg; and/or a content of available phosphorus of 0.1-200 g/kg; and/or a content of methionine of 0.1-100 g/kg; and/or a content of methionine plus cysteine of 0.1-150 g/kg; and/or a content of lysine of 0.5-50 g/kg.

In particular embodiments, the content of metabolisable energy, crude protein, calcium, phosphorus, methionine, methionine plus cysteine, and/or lysine is within any one of ranges 2, 3, 4 or 5 in Table B of WO 01/58275 (R. 2-5).

Crude protein is calculated as nitrogen (N) multiplied by a factor 6.25, i.e. Crude protein (g/kg)=N (g/kg)×6.25. The nitrogen content is determined by the Kjeldahl method (A.O.A.C., 1984, Official Methods of Analysis 14th ed., Association of Official Analytical Chemists, Washington D.C.).

Metabolisable energy can be calculated on the basis of the NRC publication Nutrient requirements in swine, ninth revised edition 1988, subcommittee on swine nutrition, committee on animal nutrition, board of agriculture, national research council. National Academy Press, Washington, D.C., pp. 2-6, and the European Table of Energy Values for Poultry Feed-stuffs, Spelderholt centre for poultry research and extension, 7361 DA Beekbergen, The Netherlands. Grafisch bedrijf Ponsen & looijen by, Wageningen. ISBN 90-71463-12-5.

The dietary content of calcium, available phosphorus and amino acids in complete animal diets is calculated on the basis of feed tables such as Veevoedertabel 1997, gegevens over chemische samenstelling, verteerbaarheid en voederwaarde van voedermiddelen, Central Veevoederbureau, Runderweg 6, 8219 pk Lelystad. ISBN 90-72839-13-7.

In a particular embodiment, the animal feed composition of the invention contains at least one vegetable protein as defined above.

The animal feed composition of the invention may also contain animal protein, such as Meat and Bone Meal, Feather meal, and/or Fish Meal, typically in an amount of 0-25%. The animal feed composition of the invention may also comprise Dried Distillers Grains with Solubles (DDGS), typically in amounts of 0-30%.

In still further particular embodiments, the animal feed composition of the invention contains 0-80% maize; and/or 0-80% sorghum; and/or 0-70% wheat; and/or 0-70% Barley; and/or 0-30% oats; and/or 0-40% soybean meal; and/or 0-25% fish meal; and/or 0-25% meat and bone meal; and/or 0-20% whey.

The animal feed may comprise vegetable proteins. In particular embodiments, the protein content of the vegetable proteins is at least 10, 20, 30, 40, 50, 60, 70, 80, or 90% (w/w). Vegetable proteins may be derived from vegetable protein sources, such as legumes and cereals, for example, materials from plants of the families Fabaceae (Leguminosae), Cruciferaceae, Chenopodiaceae, and Poaceae, such as soy bean meal, lupin meal, rapeseed meal, and combinations thereof.

In a particular embodiment, the vegetable protein source is material from one or more plants of the family Fabaceae, e.g., soybean, lupine, pea, or bean. In another particular embodiment, the vegetable protein source is material from one or more plants of the family Chenopodiaceae, e.g. beet, sugar beet, spinach or quinoa. Other examples of vegetable protein sources are rapeseed, and cabbage. In another particular embodiment, soybean is a preferred vegetable protein source. Other examples of vegetable protein sources are cereals such as barley, wheat, rye, oat, maize (corn), rice, and sorghum.

Animal diets can e.g. be manufactured as mash feed (non-pelleted) or pelleted feed. Typically, the milled feed-stuffs are mixed and sufficient amounts of essential vitamins and minerals are added according to the specifications for the species in question. Enzymes can be added as solid or liquid enzyme formulations. For example, for mash feed a solid or liquid enzyme formulation may be added before or during the ingredient mixing step. For pelleted feed the (liquid or solid) xylanase/muramidase/enzyme preparation may also be added before or during the feed ingredient step. Typically a liquid enzyme preparation comprises the xylanase, the muramidase or both the xylanase and muramidase of the invention optionally with a polyol, such as glycerol, ethylene glycol or propylene glycol, and is added after the pelleting step, such as by spraying the liquid formulation onto the pellets. The xylanase and/or muramidase may also be incorporated in a feed additive or premix.

Alternatively, the xylanase/muramidase can be prepared by freezing a mixture of liquid enzyme solution with a bulking agent such as ground soybean meal, and then lyophilizing the mixture.

In an embodiment, the composition comprises one or more additional enzymes. In an embodiment, the composition comprises one or more microbes. In an embodiment, the composition comprises one or more vitamins. In an embodiment, the composition comprises one or more minerals. In an embodiment, the composition comprises one or more amino acids. In an embodiment, the composition comprises one or more other feed ingredients.

In another embodiment, the composition comprises one or more of the polypeptides of the invention, one or more formulating agents and one or more additional enzymes. In an embodiment, the composition comprises one or more of the polypeptides of the invention, one or more formulating agents and one or more microbes. In an embodiment, the composition comprises one or more of the polypeptides of the invention, one or more formulating agents and one or more vitamins. In an embodiment, the composition comprises one or more of the polypeptides of the invention and one or more minerals. In an embodiment, the composition comprises the polypeptide of the invention, one or more formulating agents and one or more amino acids. In an embodiment, the composition comprises one or more of the polypeptides of the invention, one or more formulating agents and one or more other feed ingredients.

In a further embodiment, the composition comprises one or more of the polypeptides of the invention, one or more formulating agents and one or more components selected from the list consisting of: one or more additional enzymes; one or more microbes; one or more vitamins; one or more minerals; one or more amino acids; and one or more other feed ingredients.

The final muramidase concentration in the diet is within the range of 100 to 1000 mg enzyme protein per kg animal feed, such as 200 to 900 mg, 300 to 800 mg, 400 to 700 mg or 500 to 600 mg enzyme protein per kg animal feed, or any combination of these intervals.

The final xylanase concentration in the diet is within the range of 10 to 1000 mg enzyme protein per kg animal feed, such as 10 to 900 mg, 20 to 800 mg, 30 to 700 mg, 40 to 600 mg, 50 to 500 mg, 60 to 400 mg, 70 to 300 mg, 80 to 200 mg, 90 to 180 mg, 100 to 150 mg enzyme protein per kg animal feed, or any combination of these intervals.

For determining mg muramidase or xylanase protein per kg feed, the muramidase or xylanase is purified from the feed composition, and the specific activity of the purified muramidase or xylanase is determined using a relevant assay (see under muramidase or xylanase activity). The muramidase or xylanase activity of the feed composition as such is also determined using the same assay, and on the basis of these two determinations, the dosage in mg muramidase or xylanase protein per kg feed is calculated.

In a particular embodiment, the animal feed additive of the invention is intended for being included (or prescribed as having to be included) in animal diets or feed at levels of 0.01 to 10.0%; more particularly 0.05 to 5.0%; or 0.2 to 1.0% (% meaning g additive per 100 g feed). This is so in particular for premixes.

The same principles apply for determining mg muramidase/xylanase protein in feed additives. Of course, if a sample is available of the muramidase/xylanase used for preparing the feed additive or the feed, the specific activity is determined from this sample (no need to purify the muramidase/xylanase from the feed composition or the additive).

### Additional Enzymes

In another embodiment, the compositions described herein optionally include one or more enzymes. Enzymes can be classified on the basis of the handbook Enzyme Nomenclature from NC-IUBMB, 1992), see also the ENZYME site at the internet: http://www.expasy.ch/enzyme/. ENZYME is a repository of information relative to the nomenclature of enzymes. It is primarily based on the recommendations of the Nomenclature Committee of the International Union of Biochemistry and Molecular Biology (IUB-MB), Academic Press, Inc., 1992, and it describes each type of characterized enzyme for which an EC (Enzyme Commission) number has been provided (Bairoch A. The ENZYME database, 2000, Nucleic Acids Res 28:304-305). This IUB-MB Enzyme nomenclature is based on their substrate specificity and occasionally on their molecular mechanism; such a classification does not reflect the structural features of these enzymes.

Another classification of certain glycoside hydrolase enzymes, such as endoglucanase, galactanase, mannanase, dextranase, and galactosidase is described in Henrissat et al, “The carbohydrate-active enzymes database (CAZy) in 2013”, Nucl. Acids Res. (1 Jan. 2014) 42 (D1): D490-D495; see also www.cazy.org.

Thus the composition of the invention may also comprise at least one other enzyme selected from the group comprising of acetylxylan esterase (EC 3.1.1.23), acylglycerol lipase (EC 3.1.1.72), alpha-amylase (EC 3.2.1.1), beta-amylase (EC 3.2.1.2), arabinofuranosidase (EC 3.2.1.55), cellobiohydrolases (EC 3.2.1.91), cellulase (EC 3.2.1.4), feruloyl esterase (EC 3.1.1.73), galactanase (EC 3.2.1.89), alpha-galactosidase (EC 3.2.1.22), beta-galactosidase (EC 3.2.1.23), beta-glucanase (EC 3.2.1.6), beta-glucosidase (EC 3.2.1.21), triacylglycerol lipase (EC 3.1.1.3), lysophospholipase (EC 3.1.1.5), alpha-mannosidase (EC 3.2.1.24), beta-mannosidase (mannanase) (EC 3.2.1.25), phytase (EC 3.1.3.8, EC 3.1.3.26, EC 3.1.3.72), phospholipase A1 (EC 3.1.1.32), phospholipase A2 (EC 3.1.1.4), phospholipase D (EC 3.1.4.4), protease (EC 3.4), pullulanase (EC 3.2.1.41), pectinesterase (EC 3.1.1.11), beta-xylosidase (EC 3.2.1.37), or any combination thereof.

In a particular embodiment the composition of the invention comprises a galactanase (EC 3.2.1.89) and a beta-galactosidase (EC 3.2.1.23).

In a particular embodiment, the composition of the invention comprises a phytase (EC 3.1.3.8 or 3.1.3.26). Examples of commercially available phytases include Bio-Feed™ Phytase (Novozymes), Ronozyme® P, Ronozyme® NP and Ronozyme® HiPhos (DSM Nutritional Products), Natuphos™ (BASF), Natuphos™ E (BASF), Finase® and Quantum® Blue (AB Enzymes), OptiPhos® (Huvepharma), AveMix® Phytase (Aveve Biochem), Phyzyme® XP (Verenium/DuPont) and Axtra® PHY (DuPont). Other preferred phytases include those described in e.g. WO 98/28408, WO 00/43503, and WO 03/066847.

In a particular embodiment, the composition of the invention comprises a xylanase (EC 3.2.1.8). Examples of commercially available xylanases include Ronozyme® WX (DSM Nutritional Products), Econase® XT and Barley (AB Vista), Xylathin® (Verenium), Hostazym® X (Huvepharma), Axtra® XB (Xylanase/beta-glucanase, DuPont) and Axtra® XAP
(Xylanase/amylase/protease, DuPont), AveMix® XG 10 (xylanase/glucanase) and AveMix® 02 CS (xylanase/glucanase/pectinase, Aveve Biochem), and Naturgrain (BASF).

In a particular embodiment, the composition of the invention comprises a protease (EC 3.4). Examples of commercially available proteases include Ronozyme® ProAct (DSM Nutritional Products), Winzyme Pro Plus® (Suntaq International Limited) and Cibenza® DP100 (Novus International).

In a particular embodiment, the composition of the invention comprises an alpha-amylase (EC 3.2.1.1). Examples of commercially available alpha-amylases include Ronozyme® A and RONOZYME® RumiStar™ (DSM Nutritional Products).

In one embodiment, the composition of the invention comprises a multicomponent enzyme product, such as FRA® Octazyme (Framelco), Ronozyme® G2, Ronozyme® VP and Ronozyme® MultiGrain (DSM Nutritional Products), Rovabio® Excel or Rovabio® Advance (Adisseo).

### Eubiotics

Eubiotics are compounds which are designed to give a healthy balance of the micro-flora in the gastrointestinal tract. Eubiotics cover a number of different feed additives, such as probiotics, prebiotics, phytogenics (essential oils) and organic acids which are described in more detail below.

### Probiotics

In an embodiment, the animal feed composition further comprises one or more additional probiotic. In a particular embodiment, the animal feed composition further comprises a bacterium from one or more of the following genera: Lactobacillus, Lactococcus, Streptococcus, Bacillus, Pediococcus, Enterococcus, Leuconostoc, Carnobacterium, Propionibacterium, Bifidobacterium, Clostridium and Megasphaera or any combination thereof.

In a preferred embodiment, animal feed composition further comprises a bacterium from one or more of the following strains: Bacillus subtilis, Bacillus licheniformis, Bacillus amyloliquefaciens, Bacillus cereus, Bacillus pumilus, Bacillus polymyxa, Bacillus megaterium, Bacillus coagulans, Bacillus circulans, Enterococcus faecium, Enterococcus spp, and Pediococcus spp, Lactobacillus spp, Bifidobacterium spp, Lactobacillus acidophilus, Pediococsus acidilactici, Lactococcus lactis, Bifidobacterium bifidum, Propionibacterium thoenii, Lactobacillus farciminus, Lactobacillus rhamnosus, Clostridium butyricum, Bifidobacterium animalis ssp. animalis, Lactobacillus reuteri, Lactobacillus salivarius ssp. salivarius, Megasphaera elsdenii, Propionibacteria sp.

In a more preferred embodiment, composition, animal feed additive or animal feed further comprises a bacterium from one or more of the following strains of Bacillus subtilis: 3A-P4 (PTA-6506), 15A-P4 (PTA-6507), 22C-P1 (PTA-6508), 2084 (NRRL B-500130), LSSA01 (NRRL-B-50104), BS27 (NRRL B-501 05), BS 18 (NRRL B-50633), BS 278 (NRRL B-50634), DSM 29870, DSM 29871, DSM 32315, NRRL B-50136, NRRL B-50605, NRRL B-50606, NRRL B-50622 and PTA-7547.

In a more preferred embodiment, composition, animal feed additive or animal feed further comprises a bacterium from one or more of the following strains of Bacillus pumilus: NRRL B-50016, ATCC 700385, NRRL B-50885 or NRRL B-50886.

In a more preferred embodiment, composition, animal feed additive or animal feed further comprises a bacterium from one or more of the following strains of Bacillus lichenformis: NRRL B 50015, NRRL B-50621 or NRRL B-50623.

In a more preferred embodiment, composition, animal feed additive or animal feed further comprises a bacterium from one or more of the following strains of Bacillus amyloliquefaciens: DSM 29869, DSM 29869, NRRL B 50607, PTA-7543, PTA-7549, NRRL B-50349, NRRL B-50606, NRRL B-50013, NRRL B-50151, NRRL B-50141, NRRL B-50147 or NRRL B-50888.

The bacterial count of each of the bacterial strains in the animal feed composition is between 1×10⁴ and 1×10¹⁴ CFU/kg of dry matter, preferably between 1×10⁶ and 1×10¹² CFU/kg of dry matter, and more preferably between 1×10⁷ and 1×10¹¹ CFU/kg of dry matter. In a more preferred embodiment the bacterial count of each of the bacterial strains in the animal feed composition is between 1×10⁸ and 1×10¹⁰ CFU/kg of dry matter.

The bacterial count of each of the bacterial strains in the animal feed composition is between 1×10⁵ and 1×10¹⁵ CFU/animal/day, preferably between 1×10⁷ and 1×10¹³ CFU/animal/day, and more preferably between 1×10⁸ and 1×10¹² CFU/animal/day. In a more preferred embodiment the bacterial count of each of the bacterial strains in the animal feed composition is between 1×10⁹ and 1×10¹¹ CFU/animal/day. In one embodiment, the amount of probiotics is 0.001% to 10% by weight of the composition.

In another embodiment, the one or more bacterial strains are present in the form of a stable spore.

Examples of commercial products are Cylactin® (DSM Nutritional Products), Alterion (Adisseo), Enviva PRO (DuPont Animal Nutrition), Syncra® (mix enzyme+probiotic, DuPont Animal Nutrition), Ecobiol® and Fecinor® (Norel/Evonik) and GutCare® PY1 (Evonik).

### Prebiotics

Prebiotics are substances that induce the growth or activity of microorganisms (e.g., bacteria and fungi) that contribute to the well-being of their host. Prebiotics are typically non-digestible fiber compounds that pass undigested through the upper part of the gastrointestinal tract and stimulate the growth or activity of advantageous bacteria that colonize the large bowel by acting as substrate for them. Normally, prebiotics increase the number or activity of bifidobacteria and lactic acid bacteria in the GI tract.

Yeast derivatives (inactivated whole yeasts or yeast cell walls) can also be considered as prebiotics. They often comprise mannan-oligosaccharids, yeast beta-glucans or protein contents and are normally derived from the cell wall of the yeast, Saccharomyces cerevisiae.

In one embodiment, the amount of prebiotics is 0.001% to 10% by weight of the composition. Examples of yeast products are Yang® and Agrimos (Lallemand Animal Nutrition).

### Phytogenics

Phytogenics are a group of natural growth promoters or non-antibiotic growth promoters used as feed additives, derived from herbs, spices or other plants. Phytogenics can be single substances prepared from essential oils/extracts, essential oils/extracts, single plants and mixture of plants (herbal products) or mixture of essential oils/extracts/plants (specialized products).

Examples of phytogenics are rosemary, sage, oregano, thyme, clove, and lemongrass. Examples of essential oils are thymol, eugenol, meta-cresol, vaniline, salicylate, resorcine, guajacol, gingerol, lavender oil, ionones, irone, eucalyptol, menthol, peppermint oil, alpha-pinene; limonene, anethol, linalool, methyl dihydrojasmonate, carvacrol, propionic acid/propionate, acetic acid/acetate, butyric acid/butyrate, rosemary oil, clove oil, geraniol, terpineol, citronellol, amyl and/or benzyl salicylate, cinnamaldehyde, plant polyphenol (tannin), turmeric and curcuma extract.

In one embodiment, the amount of phytogeneics is 0.001% to 10% by weight of the composition. Examples of commercial products are Crina® (DSM Nutritional Products); Cinergy™, Biacid™, ProHacid™ Classic and ProHacid™ Advance™ (all Promivi/Cargill) and Envivo EO (DuPont Animal Nutrition).

### Organic Acids

Organic acids (C1-C7) are widely distributed in nature as normal constituents of plants or animal tissues. They are also formed through microbial fermentation of carbohydrates mainly in the large intestine. They are often used in swine and poultry production as a replacement of antibiotic growth promoters since they have a preventive effect on the intestinal problems like necrotic enteritis in chickens and Escherichia coli infection in young pigs. Organic acids can be sold as mono component or mixtures of typically 2 or 3 different organic acids. Examples of organic acids are short chain fatty acids (e.g. formic acid, acetic acid, propionic acid, butyric acid), medium chain fatty acids (e.g. caproic acid, caprylic acid, capric acid, lauric acid), di/tri-carboxylic acids (e.g. fumaric acid), hydroxy acids (e.g. lactic acid), aromatic acids (e.g. benzoic acid), citric acid, sorbic acid, malic acid, and tartaric acid or their salt (typically sodium or potassium salt such as potassium diformate or sodium butyrate).

In one embodiment, the amount of organic acid is 0.001% to 10% by weight of the composition. Examples of commercial products are VevoVitall® (DSM Nutritional Products), Amasil®, Luprisil®, Lupro-Grain®, Lupro-Cid®, Lupro-Mix® (BASF), n-Butyric Acid AF (OXEA) and Adimix Precision (Nutriad).

### Premix

The incorporation of the composition of feed additives as exemplified herein above to animal feeds, for example poultry feeds, is in practice carried out using a concentrate or a premix. A premix designates a preferably uniform mixture of one or more microingredients with diluent and/or carrier. Premixes are used to facilitate uniform dispersion of micro-ingredients in a larger mix. A premix according to the invention can be added to feed ingredients or to the drinking water as solids (for example as water soluble powder) or liquids.

### Amino Acids

The composition of the invention may further comprise one or more amino acids. Examples of amino acids which are used in animal feed are lysine, alanine, beta-alanine, threonine, methionine and tryptophan. In one embodiment, the amount of amino acid is 0.001% to 10% by weight of the composition.

### Vitamins and Minerals

In another embodiment, the animal feed may include one or more vitamins, such as one or more fat-soluble vitamins and/or one or more water-soluble vitamins. In another embodiment, the animal feed may optionally include one or more minerals, such as one or more trace minerals and/or one or more macro minerals.

Usually fat- and water-soluble vitamins, as well as trace minerals form part of a so-called premix intended for addition to the feed, whereas macro minerals are usually separately added to the feed.

Non-limiting examples of fat-soluble vitamins include vitamin A, vitamin D3, vitamin E, and vitamin K, e.g., vitamin K3.

Non-limiting examples of water-soluble vitamins include vitamin C, vitamin B12, biotin and choline, vitamin B1, vitamin B2, vitamin B6, niacin, folic acid and panthothenate, e.g., Ca-D-panthothenate.

Non-limiting examples of trace minerals include boron, cobalt, chloride, chromium, copper, fluoride, iodine, iron, manganese, molybdenum, iodine, selenium and zinc.

Non-limiting examples of macro minerals include calcium, magnesium, phosphorus, potassium and sodium.

In one embodiment, the amount of vitamins is 0.001% to 10% by weight of the composition. In one embodiment, the amount of minerals is 0.001% to 10% by weight of the composition.

The nutritional requirements of these components (exemplified with poultry and piglets/pigs) are listed in Table A of WO 01/58275. Nutritional requirement means that these components should be provided in the diet in the concentrations indicated.

In the alternative, the animal feed additive of the invention comprises at least one of the individual components specified in Table A of WO 01/58275. At least one means either of, one or more of, one, or two, or three, or four and so forth up to all thirteen, or up to all fifteen individual components. More specifically, this at least one individual component is included in the additive of the invention in such an amount as to provide an in-feed-concentration within the range indicated in column four, or column five, or column six of Table A.

In a still further embodiment, the animal feed additive of the invention comprises at least one of the below vitamins, preferably to provide an in-feed-concentration within the ranges specified in the below Table 1 (for piglet diets, and broiler diets, respectively).

TABLE 1
Typical vitamin recommendations

| Vitamin | Piglet diet | | Broiler diet | |
|---|---|---|---|---|
| Vitamin A | 10,000-15,000 | IU/kg feed | 8-12,500 | IU/kg feed |
| Vitamin D3 | 1800-2000 | IU/kg feed | 3000-5000 | IU/kg feed |
| Vitamin E | 60-100 | mg/kg feed | 150-240 | mg/kg feed |
| Vitamin K3 | 2-4 | mg/kg feed | 2-4 | mg/kg feed |
| Vitamin B1 | 2-4 | mg/kg feed | 2-3 | mg/kg feed |
| Vitamin B2 | 6-10 | mg/kg feed | 7-9 | mg/kg feed |
| Vitamin B6 | 4-8 | mg/kg feed | 3-6 | mg/kg feed |
| Vitamin B12 | 0.03-0.05 | mg/kg feed | 0.015-0.04 | mg/kg feed |
| Niacin | 30-50 | mg/kg feed | 50-80 | mg/kg feed |
| (Vitamin B3) | | | | |
| Pantothenic | 20-40 | mg/kg feed | 10-18 | mg/kg feed |
| acid | | | | |
| Folic acid | 1-2 | mg/kg feed | 1-2 | mg/kg feed |
| Biotin | 0.15-0.4 | mg/kg feed | 0.15-0.3 | mg/kg feed |
| Choline | 200-400 | mg/kg feed | 300-600 | mg/kg feed |
| chloride | | | | |

### Other Feed Ingredients

The composition of the invention may further comprise colouring agents, stabilisers, growth improving additives and aroma compounds/flavourings, polyunsaturated fatty acids (PUFAs); reactive oxygen generating species, antioxidants, anti-microbial peptides, anti-fungal polypeptides and mycotoxin management compounds.

Examples of colouring agents are carotenoids such as beta-carotene, astaxanthin, and lutein.

Examples of aroma compounds/flavourings are creosol, anethol, deca-, undeca- and/or dodeca-lactones, ionones, irone, gingerol, piperidine, propylidene phatalide, butylidene phatalide, capsaicin and tannin.

Examples of antimicrobial peptides (AMP's) are CAP18, Leucocin A, Tritrpticin, Protegrin-1, Thanatin, Defensin, Lactoferrin, Lactoferricin, and Ovispirin such as Novispirin (Robert Lehrer, 2000), Plectasins, and Statins, including the compounds and polypeptides disclosed in WO 03/044049 and WO 03/048148, as well as variants or fragments of the above that retain antimicrobial activity.

Examples of antifungal polypeptides (AFP's) are the Aspergillus giganteus, and Aspergillus niger peptides, as well as variants and fragments thereof which retain antifungal activity, as disclosed in WO 94/01459 and WO 02/090384.

Examples of polyunsaturated fatty acids are C18, C20 and C22 polyunsaturated fatty acids, such as arachidonic acid, docosohexaenoic acid, eicosapentaenoic acid and gamma-linoleic acid.

Examples of reactive oxygen generating species are chemicals such as perborate, persulphate, or percarbonate; and enzymes such as an oxidase, an oxygenase or a syntethase.

Antioxidants can be used to limit the number of reactive oxygen species which can be generated such that the level of reactive oxygen species is in balance with antioxidants.

Mycotoxins, such as deoxynivalenol, aflatoxin, zearalenone and fumonisin can be found in animal feed and can result in negative animal performance or illness. Compounds which can manage the levels of mycotoxin, such as via deactivation of the mycotoxin or via binding of the mycotoxin, can be added to the feed to ameliorate these negative effects. Examples of mycotoxin management compounds are Vitafix®, Vitafix Ultra (Nuscience), Mycofix®, Mycofix® Secure, FUMzyme®, Biomin® BBSH, Biomin® MTV (Biomin), Mold-Nil®, Toxy-Nil® and Unike® Plus (Nutriad).

### Methods of Preparing an Animal Feed

In a fifth aspect, the invention relates to a method of preparing the animal feed of the first aspect, comprising the steps of:

(a) mixing an animal feed additive with one or more protein sources and one or more energy sources;

(b) optionally steam treating the animal feed of (a) followed by pressing the steam treated mixture to form pellets; and

(c) optionally spraying a liquid formulation onto the animal feed of (a) or (b).

In one embodiment, the polypeptide having muramidase activity is added in step (a) and the polypeptide having xylanase activity is added in step (c). In one embodiment, the polypeptide having muramidase activity is added in step (c) and the polypeptide having xylanase activity is added in step (a). In one embodiment, the polypeptide having muramidase activity and the polypeptide having muramidase activity is added in step (a). In one embodiment, the polypeptide having muramidase activity and the polypeptide having muramidase activity is added in step (c).

In one embodiment, the animal feed is pelleted by steam treating the mixture of (a) to obtain a moisture content below 20% by weight of the mixture, and pressing the steam treated mixture to form pellets. In one embodiment, the animal feed is pelleted by steam treating the mixture of (a) to obtain a moisture content below 20% by weight of the mixture wherein the steam treatment is between 60° C. and 100° C. when measured at the outlet of the conditioner, and pressing the steam treated mixture to form pellets. In one embodiment, the total residence time in step b) is between 1 second and 10 minutes. In one embodiment, the temperature of the pellets after pelleting of the steam treated mixture is between 70° C. and 105° C.

### Methods of Improving Growth Performance of Animals

In a sixth aspect, the present invention further relates to a method of improving growth performance of an animal comprising administering to the animal an animal feed or animal feed additive of the invention, which comprises one or more polypeptides having xylanase activity and one or more polypeptides having muramidase activity.

Particularly, the present invention further relates to methods of improving Body Weight Gain (BWG), Feed Conversion Ratio (FCR) and/or European Production Efficiency Factor (EPEF) of an animal comprising administering to the animal the animal feed or the animal feed additive of the invention.

In a preferred embodiment, the improvement is compared to the same feed but excluding the muramidase and xylanase.

In one embodiment, the BWG is improved by at least 1%, such as by at least 1.0%, at least 1.5% or at least 2.0%. In another embodiment, the BWG is improved by between 1% and 5%, such as between 1.5% and 4%, between 2% and 3%, or any combination of these intervals. In one embodiment, the FCR is improved by at least 1%, such as by at least 1.0%, at least 1.5% or at least 2.0%. In another embodiment, the FCR is improved by between 1% and 5%, such as between 1.5% and 4%, between 2% and 3%, or any combination of these intervals.

In one embodiment, the EPEF is improved by at least 1%, such as by at least 1.0%, at least 1.5% or at least 2.0%. In another embodiment, the EPEF is improved by between 1% and 5%, such as between 1.5% and 4%, between 2% and 3%, or any combination of these intervals.

In one embodiment, the polypeptide having xylanase activity is dosed at a level of 10 to 1000 mg enzyme protein per kg animal feed, such as 10 to 900 mg, 20 to 800 mg, 30 to 700 mg, 40 to 600 mg, 50 to 500 mg, 60 to 400 mg, 70 to 300 mg, 80 to 200 mg, 90 to 180 mg, 100 to 150 mg enzyme protein per kg animal feed, or any combination of these intervals.

In one embodiment, the polypeptide having muramidase activity is dosed at a level of 100 to 1000 mg enzyme protein per kg animal feed, such as 200 to 900 mg, 300 to 800 mg, 400 to 700 mg or 500 to 600 mg enzyme protein per kg animal feed, or any combination of these intervals.

In one embodiment, the animal is a mono-gastric animal, e.g. pigs or swine (including, but not limited to, piglets, growing pigs, and sows); poultry (including but not limited to poultry, turkey, duck, quail, guinea fowl, goose, pigeon, squab, chicken, broiler, layer, pullet and chick); pet animals (such as cats and doges); fish (including but not limited to amberjack, arapaima, barb, bass, bluefish, bocachico, bream, bullhead, cachama, carp, catfish, catla, chanos, char, cichlid, cobia, cod, crappie, dorada, drum, eel, goby, goldfish, gourami, grouper, guapote, halibut, java, labeo, lai, loach, mackerel, milkfish, mojarra, mudfish, mullet, paco, pearlspot, pejerrey, perch, pike, pompano, roach, salmon, sampa, sauger, sea bass, seabream, shiner, sleeper, snakehead, snapper, snook, sole, spinefoot, sturgeon, sunfish, sweetfish, tench, terror, tilapia, trout, tuna, turbot, vendace, walleye and whitefish); and crustaceans (including but not limited to shrimps and prawns). In a more preferred embodiment, the animal is selected from the group consisting of swine, poultry, crustaceans and fish. In an even more preferred embodiment, the animal is selected from the group consisting of swine, piglet, growing pig, sow, chicken, broiler, layer, pullet and chick.

### Use in Improving Growth Performance of Animals

In a seventh aspect, the present invention further relates to use of the animal feed or animal feed additive of the invention, which comprises one or more polypeptides having xylanase activity and one or more polypeptides having muramidase activity, in improving growth performance of an animal.

Particularly, the present invention further relates to use of the animal feed or animal feed additive of the invention, which comprises one or more polypeptides having xylanase activity and one or more polypeptides having muramidase activity, in improving Body Weight Gain (BWG), Feed Conversion Ratio (FCR) and/or European Production Efficiency Factor (EPEF) of an animal.

In a preferred embodiment, the improvement is compared to the same feed but excluding the muramidase and xylanase.

In one embodiment, the BWG is improved by at least 1%, such as by at least 1.0%, at least 1.5% or at least 2.0%. In another embodiment, the BWG is improved by between 1% and 5%, such as between 1.5% and 4%, between 2% and 3%, or any combination of these intervals.

In one embodiment, the FCR is improved by at least 1%, such as by at least 1.0%, at least 1.5% or at least 2.0%. In another embodiment, the FCR is improved by between 1% and 5%, such as between 1.5% and 4%, between 2% and 3%, or any combination of these intervals.

In one embodiment, the EPEF is improved by at least 1%, such as by at least 1.0%, at least 1.5% or at least 2.0%. In another embodiment, the EPEF is improved by between 1% and 5%, such as between 1.5% and 4%, between 2% and 3%, or any combination of these intervals.

In one embodiment, the polypeptide having xylanase activity is dosed at a level of 10 to 1000 mg enzyme protein per kg animal feed, such as 10 to 900 mg, 20 to 800 mg, 30 to 700 mg, 40 to 600 mg, 50 to 500 mg, 60 to 400 mg, 70 to 300 mg, 80 to 200 mg, 90 to 180 mg, 100 to 150 mg enzyme protein per kg animal feed, or any combination of these intervals.

In one embodiment, the polypeptide having muramidase activity is dosed at a level of 100 to 1000 mg enzyme protein per kg animal feed, such as 200 to 900 mg, 300 to 800 mg, 400 to 700 mg or 500 to 600 mg enzyme protein per kg animal feed, or any combination of these intervals.

In one embodiment, the animal is a mono-gastric animal, e.g. pigs or swine (including, but not limited to, piglets, growing pigs, and sows); poultry (including but not limited to poultry, turkey, duck, quail, guinea fowl, goose, pigeon, squab, chicken, broiler, layer, pullet and chick); pet animals (such as cats and doges); fish (including but not limited to amberjack, arapaima, barb, bass, bluefish, bocachico, bream, bullhead, cachama, carp, catfish, catla, chanos, char, cichlid, cobia, cod, crappie, dorada, drum, eel, goby, goldfish, gourami, grouper, guapote, halibut, Java, labeo, lai, loach, mackerel, milkfish, mojarra, mudfish, mullet, paco, pearlspot, pejerrey, perch, pike, pompano, roach, salmon, sampa, sauger, sea bass, seabream, shiner, sleeper, snakehead, snapper, snook, sole, spinefoot, sturgeon, sunfish, sweetfish, tench, terror, tilapia, trout, tuna, turbot, vendace, walleye and whitefish); and crustaceans (including but not limited to shrimps and prawns). In a more preferred embodiment, the animal is selected from the group consisting of swine, poultry, crustaceans and fish. In an even more preferred embodiment, the animal is selected from the group consisting of swine, piglet, growing pig, sow, chicken, broiler, layer, pullet and chick.

The present invention will be further illustrated by the following examples.

### EXAMPLES

### Example 1: Determination of Muramidase Activity

The activity of muramidase was determined by measuring the decrease (drop) in absorbance/optical density of a solution of suspended Micrococcus lysodeikticus ATTC No. 4698 (Sigma-Aldrich M3770) measured in a microplate reader (Tecan Infinite M200) at 450 nm.

Preparation of Micrococcus lysodeikticus Substrate

Before use the cells were suspended in deionized water to a concentration of 10 mg cells/mL and the absorbance/optical density (OD) at 450 nm was measured. The cell suspension was then adjusted so that the cell concentration in the turbidity assay (180 μL buffer+20 μL sample+20 μL substrate) equaled an OD450=1 0.0. The adjusted cell suspension was then stored at ambient temperature before use. Suspended cells were used within 3 hours.

### Preparation of Citric Acid-Phosphate Buffer pH 4

61.45 mL 0.1 M citric acid was mixed with 38.55 mL 0.2 M disodium hydrogen phosphate, and the pH was adjusted with hydrochloric acid or sodium hydroxide to pH 4.

### Measurement of Muramidase Antimicrobial Activity in the Turbidity Assay

The muramidase sample to be measured was diluted to a concentration of 50 mg enzyme protein/L in deionized water, and kept on ice until use. In a 96 well microtiter plate (Nunc) 180 μL citric acid-phosphate buffer pH 4 and 20 μL of the diluted muramidase sample was added and kept cold (5° C.). To start the activity measurement 20 μL of the substrate (Micrococcus lysodeikticus) was added to each well, and kinetic measurement of absorbance at 450 nm was initiated for 1 hour at 37° C. in a microplate reader. The measured absorbance at 450 nm was monitored for each well and over time a drop in absorbance was seen if the muramidase has muramidase activity.

Following incubation, the muramidase activity against Micrococcus lysodeikticus was determined as Δ absorbance at 450 nm (start value−end value) of each well after 1 hour. Significance was calculated using Dunnett's with control test p level 0.05 in JMP® version 12.1.0 statistical software package from SAS Institute Inc.

### Example 2: Cloning, Expression and Purification of the Muramidases

The GH25 muramidases of SEQ ID NO: 1 to SEQ ID NO: 2 were cloned and expressed as described in example 2 of WO 2013/076253. The GH25 muramidase of SEQ ID NO: 3 may be cloned using basic molecular techniques (Ausubel et al., 2003, Curr. Prot. Mol. Biol., John Wiley & Sons, Cambridge, USA; Christgau et al. 1995, Curr. Genet. 27, 135-141). The GH25 muramidase of SEQ ID NO: 4 may be cloned and expressed as described in WO2009/102755. The GH25 muramidase of SEQ ID NO: 5 was cloned and expressed as described in WO2005/080559. The GH25 muramidases of SEQ ID NO: 6 to SEQ ID NO: 59 were cloned and expressed as described in PCT/CN2017/075978. The GH25 muramidases of SEQ ID NO: 60 to SEQ ID NO: 62 were cloned and expressed as described in PCT/CN2017/075960. The GH24 muramidases of SEQ ID NO: 63 to SEQ ID NO: 71 were cloned and expressed as described in WO2017/000922.

### Example 3: Cloning, Expression and Purification of the Xylanases

The GH10 xylanase of SEQ ID NO: 72 was cloned and expressed as described in WO1994/021785. The GH10 xylanase of SEQ ID NO: 73 may be cloned and expressed as described in Appl. Environ. Microbiol. 1987, 53(4):644. The GH10 xylanase of SEQ ID NO: 74 was cloned and expressed as described in WO2005/059084. The GH10 xylanase of SEQ ID NO: 75 may be cloned and expressed as described in WO2013/068550. The GH10 xylanase of SEQ ID NO: 76 and SEQ ID NO: 77 were cloned and expressed as described in WO2016/095856. The GH10 xylanase of SEQ ID NO: 78 may be cloned and expressed as described in WO2001/42433. The GH10 xylanase of SEQ ID NO: 78 may be cloned and expressed as described in WO2002/24926.

The GH11 xylanase of SEQ ID NO: 80 was cloned and expressed as described in WO2009/018537. The GH11 xylanase of SEQ ID NO: 81 was cloned and expressed as described in WO2016/095856. The GH11 xylanase of SEQ ID NO: 82 may be cloned and expressed as described in WO1999/57325. The GH11 xylanase of SEQ ID NO: 83 may be cloned and expressed as described in WO2001/66711. The GH11 xylanase of SEQ ID NO: 84 may be cloned and expressed as described in WO2002/38746. The GH11 xylanase of SEQ ID NO: 85 may be cloned and expressed as described in WO2005100557. The GH11 xylanase of SEQ ID NO: 86 and SEQ ID NO: 87 may be cloned and expressed as described in WO1993/24621. The GH11 xylanase of SEQ ID NO: 88 may be cloned and expressed as described in U.S. Pat. No. 5,306,633. The GH11 xylanase of SEQ ID NO: 89 may be cloned and expressed as described in WO2007/146944.

The GH5_21 and GH5_35 xylanases of SEQ ID NO: 111 to 116 were cloned and expressed as described in WO2016/005522. The GH30_8 xylanases of SEQ ID NO: 111 to 116 were cloned and expressed as described in PCT/EP2017/065336. The GH30_8 xylanases of SEQ ID NO: 117 to 126 were cloned and expressed as described in WO2017/103159.

### Example 4: In Vivo Broiler Trial

### Materials and Methods

The trial was performed at the Research Center for Animal Nutrition (DSM Nutritional Products France, F-68305 Village-Neuf) according to the official French guidelines for experiments with live animals. Day-old male broiler chickens (“ROSS PM3”), were supplied by a commercial hatchery (Joseph Grelier S. A., Elevage avicole de la Bohadière, F-49290 Saint-Laurent de la Plaine, France).

### Animals and Housing

On the day of arrival (day 1), the chickens were divided by weight into groups of 18 birds. Each group was placed in one floor-pen littered with wood shavings and allocated to one of the different treatments.

Each treatment was replicated with 8 groups. The chickens were housed in an environmentally controlled room. The room temperature was adapted to the age of the birds. In the first few days an additional infra-red electric heating lamp was placed in each pen. Moreover, in the first week feed was offered to the birds as crumbled pellets, afterwards as pelleted feed. The birds had free access to feed and water.

### Feeding and Treatments

The experimental diets (Starter and Grower) were based on soybean meal, wheat and rye (12%) as main ingredients (Table 2). The diets were formulated to contain 222 g crude protein and 12.5 MJ/kg ME_{N} for the starter period and 204 g crude protein and 12.7 MJ/kg ME_{N} for the grower period. The basal diets did not contain any coccidiostat.

TABLE 2
Composition and nutrient contents of the basal experimental diets

| Ingredients (%) | Starter (d 1-22) | Grower (d 22-36) |
|---|---|---|
| Soybean meal | 37.65 | 32.80 |
| Corn | 22.35 | 23.05 |
| Wheat | 20.00 | 24.20 |
| Rye | 12.00 | 12.00 |
| Soya oil | 3.90 | 4.00 |
| DL-Methionine | 0.20 | 0.10 |
| L-Lysine | — | — |
| NaCl | 0.20 | 0.15 |
| DCP | 1.80 | 1.80 |
| CaCO3 | 0.90 | 0.90 |
| Premix¹ | 1.00 | 1.00 |
| Calculated content | | |
| Crude protein (%) | 22.2 | 20.4 |
| Metabolizable energy (MJ/kg)² | 12.5 | 12.6 |
| Analyzed content | | |
| Crude protein (%) | 22.4 | 20.1 |
| Metabolizable energy (MJ/kg)³ | 12.8 | 12.7 |

¹Vitamin-mineral premix provided per kilogram of diet: Vitamin A: 10,000 I.U.; vitamin E: 40 I.U.; vitamin K3: 3.0 mg; vitamin C: 100 mg; vitamin B1: 2.50 mg; vitamin B2: 8.00 mg; vitamin B6: 5.00 mg; vitamin B12: 0.03 mg; niacin: 50.0 mg; pantothenate calcium: 12.0 mg; folic acid: 1.50 mg; biotin 0.15 mg; cholin: 450 mg; ethoxyquine: 54 mg; Na: 1.17 g; Mg: 0.8 g; Mn: 80 mg; Fe: 60 mg; Cu: 30 mg; Zn: 54 mg; I: 1.24 mg; Co: 0.6 mg; Se: 0.3 mg
¹Without coccidiostat;
²Calculated with EC-equation;
³Calculated with EC-equation based on analysed crude nutrients

The diets were fed either unsupplemented or supplemented with the GH25 muramidase (SEQ ID NO: 1) and/or Ronozyme WX as follows:

TABLE 3
Feeding Regime

| | Treatment | Dosage | |
|---|---|---|---|
| A | Control (NC) | — | |
| B | Muramidase GH25 new T. reesei | 25 000 | LSU/kg |
| C | Muramidase GH25 new T. reesei | 35 000 | LSU /kg |
| D | Ronozyme WX | 150 | mg/kg |

| | | |
|---|---|---|
| E | Muramidase GH25 new T. reesei + | 25 000 LSU/kg + 150 mg/kg |
| | Ronozyme WX | |
| F | Muramidase GH25 new T. reesei + | 35 000 LSU/kg + 150 mg/kg |
| | Ronozyme WX | |

### Experimental Parameters and Analyses

For the two experiments, the birds were weighed (as replicate group) on days 1, 22 and 36. The feed consumption for the intermediate periods was determined. Body weight gain, feed conversion ratio (feed/gain) and EPEF were calculated.

The analyses of the nutrient content in the feed samples were performed according to standard methods (VDLUFA 1976). Nitrogen analysis was carried out with a Leco N analyzer (CP=N*6.25).

### Statistical Analysis

For the statistical evaluation of performance data, a one-factorial analysis of variance (factor: treatment) was carried out. The software ‘Stat Box Pro Agri’, version 7.1.9 (Grimmer soft, 1985-2011) was used. Where significant treatment effects (p<0.05) were indicated, the differences among treatment means were subsequently determined with the Newman-Keuls test.

### Results and Discussion

The results of the growth performance are summarized in table 4.

TABLE 4
Growth performance data¹ of male broiler chickens fed graded inclusion
levels of microbial muramidase and xylanse (Ronozyme WX)

| | | BWG (g/bird) | FCR | Mortality (%) | EPEF |
|---|---|---|---|---|---|
| A | NC | 2467 | 1.621 | 12.5 | 370 |
| B | GH25-25 | 2484 | 1.593 | 10.4 | 388 |
| C | GH25-35 | 2463 | 1.576 | 12.5 | 380 |
| D | WX | 2456 | 1.600 | 8.3 | 391 |
| E | GH25-25 + WX | 2547 | 1.600 | 10.4 | 396 |
| F | GH25-35 + WX | 2516 | 1.541 | 10.4 | 406 |

Weight Gain
GH25+WX: BWG was improved by 3.2% and 2.0% respectively, with the combination of Ronozyme WX with either GH25 at 25000 or 35 000 LSU, compared to NC diet

FCR
GH25: Improvement by 1.7%, 2.7% with GH25 at 25 000 LSU and 35000 LSU
Ronozyme WX: Improvement by 1.3%
GH25+WX: An additive effect by 2.2% was obtained when GH25 at 35 000 LSU was combined with Ronozyme WX, compared to the supplementation of GH25 at 35 000 LSU alone

EPEF
GH25: Improvement by 4.9%, 2.7% with GH25 at 25 000 LSU and 35000 LSU
Ronozyme WX: Improvement by 5.7%
GH25+WX: EPEF was also improved by 7% and 9.7% respectively with the combination of Ronozyme WX with either GH25 at 25000 or 35 000 LSU, compared to NC diet.

### CONCLUSION

The results obtained in the study showed that the inclusion of the xylanse in combination with the microbial muramidase was effective in improving the growth performance of broilers.

The invention described and claimed herein is not to be limited in scope by the specific aspects herein disclosed, since these aspects are intended as illustrations of several aspects of the invention. Any equivalent aspects are intended to be within the scope of this invention. Indeed, various modifications of the invention in addition to those shown and described herein will become apparent to those skilled in the art from the foregoing description. Such modifications are also intended to fall within the scope of the appended claims. In the case of conflict, the present disclosure including definitions will control.

1. An animal feed comprising an animal feed additive, one or more protein sources and one or more energy sources characterised in the animal feed or the animal feed additive further comprises one or more polypeptides having xylanase activity and one or more fungal polypeptides having muramidase activity.

2. The animal feed of claim 1, wherein the polypeptide having muramidase activity is a fungal GH24 muramidase or GH25 muramidase.

3. The animal feed of claim 1, wherein the polypeptide having xylanase activity is a GH10 xylanase, GH11 xylanase, GH5 xylanase or GH30 xylanase.

4. The animal feed of claim 1, wherein the polypeptide having muramidase activity degrades cell wall debris from Lactobacillus johnsonii.

5. The animal feed of claim 1, wherein the polypeptide having muramidase activity is selected from the group consisting of:
(a) a polypeptide having at least 80%, e.g., at least 85%, at least 90%, at least 95%, or 100% sequence identity to SEQ ID NO: 1;
(b) a polypeptide having at least 80%, e.g., at least 85%, at least 90%, at least 95%, or 100% sequence identity to SEQ ID NO: 2;
(c) a polypeptide having at least 80%, e.g., at least 85%, at least 90%, at least 95%, or 100% sequence identity to SEQ ID NO: 3;
(d) a polypeptide having at least 80%, e.g., at least 85%, at least 90%, at least 95%, or 100% sequence identity to SEQ ID NO: 4;
(e) a polypeptide having at least 80%, e.g., at least 85%, at least 90%, at least 95%, or 100% sequence identity to SEQ ID NO: 5;
(f) a polypeptide having at least 80%, e.g., at least 85%, at least 90%, at least 95%, or 100% sequence identity to SEQ ID NO: 6;
(g) a polypeptide having at least 80%, e.g., at least 85%, at least 90%, at least 95%, or 100% sequence identity to SEQ ID NO: 7;
(h) a polypeptide having at least 80%, e.g., at least 85%, at least 90%, at least 95%, or 100% sequence identity to SEQ ID NO: 8;
(i) a polypeptide having at least 80%, e.g., at least 85%, at least 90%, at least 95%, or 100% sequence identity to SEQ ID NO: 9;
(j) a polypeptide having at least 80%, e.g., at least 85%, at least 90%, at least 95%, or 100% sequence identity to SEQ ID NO: 10;
(k) a polypeptide having at least 80%, e.g., at least 85%, at least 90%, at least 95%, or 100% sequence identity to SEQ ID NO: 11;
(l) a polypeptide having at least 80%, e.g., at least 85%, at least 90%, at least 95%, or 100% sequence identity to SEQ ID NO: 12;
(m) a polypeptide having at least 80%, e.g., at least 85%, at least 90%, at least 95%, or 100% sequence identity to SEQ ID NO: 13;
(n) a polypeptide having at least 80%, e.g., at least 85%, at least 90%, at least 95%, or 100% sequence identity to SEQ ID NO: 14;
(o) a polypeptide having at least 80%, e.g., at least 85%, at least 90%, at least 95%, or 100% sequence identity to SEQ ID NO: 15;
(p) a polypeptide having at least 80%, e.g., at least 85%, at least 90%, at least 95%, or 100% sequence identity to SEQ ID NO: 16;
(q) a polypeptide having at least 80%, e.g., at least 85%, at least 90%, at least 95%, or 100% sequence identity to SEQ ID NO: 17;
(r) a polypeptide having at least 80%, e.g., at least 85%, at least 90%, at least 95%, or 100% sequence identity to SEQ ID NO: 18;
(s) a polypeptide having at least 80%, e.g., at least 85%, at least 90%, at least 95%, or 100% sequence identity to SEQ ID NO: 19;
(t) a polypeptide having at least 80%, e.g., at least 85%, at least 90%, at least 95%, or 100% sequence identity to SEQ ID NO: 20;
(u) a polypeptide having at least 80%, e.g., at least 85%, at least 90%, at least 95%, or 100% sequence identity to SEQ ID NO: 21;
(v) a polypeptide having at least 80%, e.g., at least 85%, at least 90%, at least 95%, or 100% sequence identity to SEQ ID NO: 22;
(w) a polypeptide having at least 80%, e.g., at least 85%, at least 90%, at least 95%, or 100% sequence identity to SEQ ID NO: 23;
(x) a polypeptide having at least 80%, e.g., at least 85%, at least 90%, at least 95%, or 100% sequence identity to SEQ ID NO: 24;
(y) a polypeptide having at least 80%, e.g., at least 85%, at least 90%, at least 95%, or 100% sequence identity to SEQ ID NO: 25;
(z) a polypeptide having at least 80%, e.g., at least 85%, at least 90%, at least 95%, or 100% sequence identity to SEQ ID NO: 26;
(aa) a polypeptide having at least 80%, e.g., at least 85%, at least 90%, at least 95%, or 100% sequence identity to SEQ ID NO: 27;
(ab) a polypeptide having at least 80%, e.g., at least 85%, at least 90%, at least 95%, or 100% sequence identity to SEQ ID NO: 28;
(ac) a polypeptide having at least 80%, e.g., at least 85%, at least 90%, at least 95%, or 100% sequence identity to SEQ ID NO: 29;
(ad) a polypeptide having at least 80%, e.g., at least 85%, at least 90%, at least 95%, or 100% sequence identity to SEQ ID NO: 30;
(ae) a polypeptide having at least 80%, e.g., at least 85%, at least 90%, at least 95%, or 100% sequence identity to SEQ ID NO: 31;
(af) a polypeptide having at least 80%, e.g., at least 85%, at least 90%, at least 95%, or 100% sequence identity to SEQ ID NO: 32;
(ag) a polypeptide having at least 80%, e.g., at least 85%, at least 90%, at least 95%, or 100% sequence identity to SEQ ID NO: 33;
(ah) a polypeptide having at least 80%, e.g., at least 85%, at least 90%, at least 95%, or 100% sequence identity to SEQ ID NO: 34;
(ai) a polypeptide having at least 80%, e.g., at least 85%, at least 90%, at least 95%, or 100% sequence identity to SEQ ID NO: 35;
(aj) a polypeptide having at least 80%, e.g., at least 85%, at least 90%, at least 95%, or 100% sequence identity to SEQ ID NO: 36;
(ak) a polypeptide having at least 80%, e.g., at least 85%, at least 90%, at least 95%, or 100% sequence identity to SEQ ID NO: 37;
(al) a polypeptide having at least 80%, e.g., at least 85%, at least 90%, at least 95%, or 100% sequence identity to SEQ ID NO: 38;
(am) a polypeptide having at least 80%, e.g., at least 85%, at least 90%, at least 95%, or 100% sequence identity to SEQ ID NO: 39;
(an) a polypeptide having at least 80%, e.g., at least 85%, at least 90%, at least 95%, or 100% sequence identity to SEQ ID NO: 40;
(ao) a polypeptide having at least 80%, e.g., at least 85%, at least 90%, at least 95%, or 100% sequence identity to SEQ ID NO: 41;
(ap) a polypeptide having at least 80%, e.g., at least 85%, at least 90%, at least 95%, or 100% sequence identity to SEQ ID NO: 42;
(aq) a polypeptide having at least 80%, e.g., at least 85%, at least 90%, at least 95%, or 100% sequence identity to SEQ ID NO: 43;
(ar) a polypeptide having at least 80%, e.g., at least 85%, at least 90%, at least 95%, or 100% sequence identity to SEQ ID NO: 44;
(as) a polypeptide having at least 80%, e.g., at least 85%, at least 90%, at least 95%, or 100% sequence identity to SEQ ID NO: 45;
(at) a polypeptide having at least 80%, e.g., at least 85%, at least 90%, at least 95%, or 100% sequence identity to SEQ ID NO: 46;
(au) a polypeptide having at least 80%, e.g., at least 85%, at least 90%, at least 95%, or 100% sequence identity to SEQ ID NO: 47;
(av) a polypeptide having at least 80%, e.g., at least 85%, at least 90%, at least 95%, or 100% sequence identity to SEQ ID NO: 48;
(aw) a polypeptide having at least 80%, e.g., at least 85%, at least 90%, at least 95%, or 100% sequence identity to SEQ ID NO: 49;
(ax) a polypeptide having at least 80%, e.g., at least 85%, at least 90%, at least 95%, or 100% sequence identity to SEQ ID NO: 50;
(ay) a polypeptide having at least 80%, e.g., at least 85%, at least 90%, at least 95%, or 100% sequence identity to SEQ ID NO: 51;
(az) a polypeptide having at least 80%, e.g., at least 85%, at least 90%, at least 95%, or 100% sequence identity to SEQ ID NO: 52;
(ba) a polypeptide having at least 80%, e.g., at least 85%, at least 90%, at least 95%, or 100% sequence identity to SEQ ID NO: 53;
(bb) a polypeptide having at least 80%, e.g., at least 85%, at least 90%, at least 95%, or 100% sequence identity to SEQ ID NO: 54;
(bc) a polypeptide having at least 80%, e.g., at least 85%, at least 90%, at least 95%, or 100% sequence identity to SEQ ID NO: 55;
(bd) a polypeptide having at least 80%, e.g., at least 85%, at least 90%, at least 95%, or 100% sequence identity to SEQ ID NO: 56;
(be) a polypeptide having at least 80%, e.g., at least 85%, at least 90%, at least 95%, or 100% sequence identity to SEQ ID NO: 57;
(bf) a polypeptide having at least 80%, e.g., at least 85%, at least 90%, at least 95%, or 100% sequence identity to SEQ ID NO: 58;
(bg) a polypeptide having at least 80%, e.g., at least 85%, at least 90%, at least 95%, or 100% sequence identity to SEQ ID NO: 59;
(bh) a polypeptide having at least 80%, e.g., at least 85%, at least 90%, at least 95%, or 100% sequence identity to SEQ ID NO: 60;
(bi) a polypeptide having at least 80%, e.g., at least 85%, at least 90%, at least 95%, or 100% sequence identity to SEQ ID NO: 61;
(bj) a polypeptide having at least 80%, e.g., at least 85%, at least 90%, at least 95%, or 100% sequence identity to SEQ ID NO: 62;
(bk) a polypeptide having at least 80%, e.g., at least 85%, at least 90%, at least 95%, or 100% sequence identity to SEQ ID NO: 63;
(bl) a polypeptide having at least 80%, e.g., at least 85%, at least 90%, at least 95%, or 100% sequence identity to SEQ ID NO: 64;
(bm) a polypeptide having at least 80%, e.g., at least 85%, at least 90%, at least 95%, or 100% sequence identity to SEQ ID NO: 65;
(bn) a polypeptide having at least 80%, e.g., at least 85%, at least 90%, at least 95%, or 100% sequence identity to SEQ ID NO: 66;
(bo) a polypeptide having at least 80%, e.g., at least 85%, at least 90%, at least 95%, or 100% sequence identity to SEQ ID NO: 67;
(bp) a polypeptide having at least 80%, e.g., at least 85%, at least 90%, at least 95%, or 100% sequence identity to SEQ ID NO: 68;
(bq) a polypeptide having at least 80%, e.g., at least 85%, at least 90%, at least 95%, or 100% sequence identity to SEQ ID NO: 69;
(br) a polypeptide having at least 80%, e.g., at least 85%, at least 90%, at least 95%, or 100% sequence identity to SEQ ID NO: 70;
(bs) a polypeptide having at least 80%, e.g., at least 85%, at least 90%, at least 95%, or 100% sequence identity to SEQ ID NO: 71;
(bt) a variant of SEQ ID NO: 1, SEQ ID NO: 2, SEQ ID NO: 3, SEQ ID NO: 4, SEQ ID NO: 5, SEQ ID NO: 6, SEQ ID NO: 7, SEQ ID NO: 8, SEQ ID NO: 9, SEQ ID NO: 10, SEQ ID NO: 11, SEQ ID NO: 12, SEQ ID NO: 13, SEQ ID NO: 14, SEQ ID NO: 15, SEQ ID NO: 16, SEQ ID NO: 17, SEQ ID NO: 18, SEQ ID NO: 19, SEQ ID NO: 20, SEQ ID NO: 21, SEQ ID NO: 22, SEQ ID NO: 23, SEQ ID NO: 24, SEQ ID NO: 25, SEQ ID NO: 26, SEQ ID NO: 27, SEQ ID NO: 28, SEQ ID NO: 29, SEQ ID NO: 30, SEQ ID NO: 31, SEQ ID NO: 32, SEQ ID NO: 33, SEQ ID NO: 34, SEQ ID NO: 35, SEQ ID NO: 36, SEQ ID NO: 37, SEQ ID NO: 38, SEQ ID NO: 39, SEQ ID NO: 40, SEQ ID NO: 41, SEQ ID NO: 42, SEQ ID NO: 43, SEQ ID NO: 44, SEQ ID NO: 45, SEQ ID NO: 46, SEQ ID NO: 47, SEQ ID NO: 48, SEQ ID NO: 49, SEQ ID NO: 50, SEQ ID NO: 51, SEQ ID NO: 52, SEQ ID NO: 53, SEQ ID NO: 54, SEQ ID NO: 55, SEQ ID NO: 56, SEQ ID NO: 57, SEQ ID NO: 58, SEQ ID NO: 59, SEQ ID NO: 60, SEQ ID NO: 61, SEQ ID NO: 62, SEQ ID NO: 63, SEQ ID NO: 64, SEQ ID NO: 65, SEQ ID NO: 66, SEQ ID NO: 67, SEQ ID NO: 68, SEQ ID NO: 69, SEQ ID NO: 70 or SEQ ID NO: 71 comprising one or more amino acid substitutions (preferably conservative substitutions), and/or one or more amino acid deletions, and/or one or more amino acid insertions or any combination thereof in 1, 2, 3, 4, 5, 6, 7, 8, 9 or 10 positions;
(bu) a polypeptide comprising the polypeptide of (a), (b), (c), (d), (e), (f), (g), (h), (i), (j), (k), (1), (m), (n), (o), (p), (q), (r), (s), (t), (u), (v), (w), (x), (y), (z), (aa), (ab), (ac), (ad), (ae), (af), (ag), (ah), (ai), (aj), (ak), (al), (am), (an), (ao), (ap), (aq), (ar), (as), (at), (au), (av), (aw), (ax), (ay), (az), (ba), (bb), (bc), (bd), (be), (bf), (bg), (bh), (bi), (bj), (bk), (bl), (bm), (bn), (bo), (bp), (bq), (br), (bs) or (bt) and a N-terminal and/or C-terminal extension of between 1 and 10 amino acids; and
(bv) a fragment of a polypeptide of (a), (b), (c), (d), (e), (f), (g), (h), (i), (j), (k), (1), (m), (n), (o), (p), (q), (r), (s), (t), (u), (v), (w), (x), (y), (z), (aa), (ab), (ac), (ad), (ae), (af), (ag), (ah), (ai), (aj), (ak), (al), (am), (an), (ao), (ap), (aq), (ar), (as), (at), (au), (av), (aw), (ax), (ay), (az), (ba), (bb), (bc), (bd), (be), (bf), (bg), (bh), (bi), (bj), (bk), (bl), (bm), (bn), (bo), (bp), (bq), (br), (bs) or (bt) having muramidase activity and having at least 90% of the length of the mature polypeptide.

6. The animal feed of claim 1, wherein the polypeptide having xylanase activity is selected from the group consisting of:
(a) a polypeptide having at least 80%, e.g., at least 85%, at least 90%, at least 95%, or 100% sequence identity to SEQ ID NO: 72;
(b) a polypeptide having at least 80%, e.g., at least 85%, at least 90%, at least 95%, or 100% sequence identity to SEQ ID NO: 73;
(c) a polypeptide having at least 80%, e.g., at least 85%, at least 90%, at least 95%, or 100% sequence identity to SEQ ID NO: 74;
(d) a polypeptide having at least 80%, e.g., at least 85%, at least 90%, at least 95%, or 100% sequence identity to SEQ ID NO: 75;
(e) a polypeptide having at least 80%, e.g., at least 85%, at least 90%, at least 95%, or 100% sequence identity to SEQ ID NO: 76;
(f) a polypeptide having at least 80%, e.g., at least 85%, at least 90%, at least 95%, or 100% sequence identity to SEQ ID NO: 77;
(g) a polypeptide having at least 80%, e.g., at least 85%, at least 90%, at least 95%, or 100% sequence identity to SEQ ID NO: 78;
(h) a polypeptide having at least 80%, e.g., at least 85%, at least 90%, at least 95%, or 100% sequence identity to SEQ ID NO: 79;
(i) a polypeptide having at least 80%, e.g., at least 85%, at least 90%, at least 95%, or 100% sequence identity to SEQ ID NO: 80;
(j) a polypeptide having at least 80%, e.g., at least 85%, at least 90%, at least 95%, or 100% sequence identity to SEQ ID NO: 81;
(k) a polypeptide having at least 80%, e.g., at least 85%, at least 90%, at least 95%, or 100% sequence identity to SEQ ID NO: 82;
(l) a polypeptide having at least 80%, e.g., at least 85%, at least 90%, at least 95%, or 100% sequence identity to SEQ ID NO: 83;
(m) a polypeptide having at least 80%, e.g., at least 85%, at least 90%, at least 95%, or 100% sequence identity to SEQ ID NO: 84;
(n) a polypeptide having at least 80%, e.g., at least 85%, at least 90%, at least 95%, or 100% sequence identity to SEQ ID NO: 85;
(o) a polypeptide having at least 80%, e.g., at least 85%, at least 90%, at least 95%, or 100% sequence identity to SEQ ID NO: 86;
(p) a polypeptide having at least 80%, e.g., at least 85%, at least 90%, at least 95%, or 100% sequence identity to SEQ ID NO: 87;
(q) a polypeptide having at least 80%, e.g., at least 85%, at least 90%, at least 95%, or 100% sequence identity to SEQ ID NO: 88;
(r) a polypeptide having at least 80%, e.g., at least 85%, at least 90%, at least 95%, or 100% sequence identity to SEQ ID NO: 89;
(s) a polypeptide having at least 80%, e.g., at least 85%, at least 90%, at least 95%, or 100% sequence identity to SEQ ID NO: 90;
(t) a polypeptide having at least 80%, e.g., at least 85%, at least 90%, at least 95%, or 100% sequence identity to SEQ ID NO: 91;
(u) a polypeptide having at least 80%, e.g., at least 85%, at least 90%, at least 95%, or 100% sequence identity to SEQ ID NO: 92;
(v) a polypeptide having at least 80%, e.g., at least 85%, at least 90%, at least 95%, or 100% sequence identity to SEQ ID NO: 93;
(w) a polypeptide having at least 80%, e.g., at least 85%, at least 90%, at least 95%, or 100% sequence identity to SEQ ID NO: 94;
(x) a polypeptide having at least 80%, e.g., at least 85%, at least 90%, at least 95%, or 100% sequence identity to SEQ ID NO: 95;
(y) a polypeptide having at least 80%, e.g., at least 85%, at least 90%, at least 95%, or 100% sequence identity to SEQ ID NO: 96;
(z) a polypeptide having at least 80%, e.g., at least 85%, at least 90%, at least 95%, or 100% sequence identity to SEQ ID NO: 97;
(aa) a polypeptide having at least 80%, e.g., at least 85%, at least 90%, at least 95%, or 100% sequence identity to SEQ ID NO: 98;
(ab) a polypeptide having at least 80%, e.g., at least 85%, at least 90%, at least 95%, or 100% sequence identity to SEQ ID NO: 99;
(ac) a polypeptide having at least 80%, e.g., at least 85%, at least 90%, at least 95%, or 100% sequence identity to SEQ ID NO: 100;
(ad) a polypeptide having at least 80%, e.g., at least 85%, at least 90%, at least 95%, or 100% sequence identity to SEQ ID NO: 101;
(ae) a polypeptide having at least 80%, e.g., at least 85%, at least 90%, at least 95%, or 100% sequence identity to SEQ ID NO: 102;
(af) a polypeptide having at least 80%, e.g., at least 85%, at least 90%, at least 95%, or 100% sequence identity to SEQ ID NO: 103;
(ag) a polypeptide having at least 80%, e.g., at least 85%, at least 90%, at least 95%, or 100% sequence identity to SEQ ID NO: 104;
(ah) a polypeptide having at least 80%, e.g., at least 85%, at least 90%, at least 95%, or 100% sequence identity to SEQ ID NO: 105;
(al) a polypeptide having at least 80%, e.g., at least 85%, at least 90%, at least 95%, or 100% sequence identity to SEQ ID NO: 106;
(aj) a polypeptide having at least 80%, e.g., at least 85%, at least 90%, at least 95%, or 100% sequence identity to SEQ ID NO: 107;
(ak) a polypeptide having at least 80%, e.g., at least 85%, at least 90%, at least 95%, or 100% sequence identity to SEQ ID NO: 108;
(al) a polypeptide having at least 80%, e.g., at least 85%, at least 90%, at least 95%, or 100% sequence identity to SEQ ID NO: 109;
(am) a polypeptide having at least 80%, e.g., at least 85%, at least 90%, at least 95%, or 100% sequence identity to SEQ ID NO: 110;
(an) a polypeptide having at least 80%, e.g., at least 85%, at least 90%, at least 95%, or 100% sequence identity to SEQ ID NO: 111;
(ao) a polypeptide having at least 80%, e.g., at least 85%, at least 90%, at least 95%, or 100% sequence identity to SEQ ID NO: 112;
(ap) a polypeptide having at least 80%, e.g., at least 85%, at least 90%, at least 95%, or 100% sequence identity to SEQ ID NO: 113;
(aq) a polypeptide having at least 80%, e.g., at least 85%, at least 90%, at least 95%, or 100% sequence identity to SEQ ID NO: 114;
(ar) a polypeptide having at least 80%, e.g., at least 85%, at least 90%, at least 95%, or 100% sequence identity to SEQ ID NO: 115;
(as) a polypeptide having at least 80%, e.g., at least 85%, at least 90%, at least 95%, or 100% sequence identity to SEQ ID NO: 116;
(at) a polypeptide having at least 80%, e.g., at least 85%, at least 90%, at least 95%, or 100% sequence identity to SEQ ID NO: 117;
(au) a polypeptide having at least 80%, e.g., at least 85%, at least 90%, at least 95%, or 100% sequence identity to SEQ ID NO: 118;
(av) a polypeptide having at least 80%, e.g., at least 85%, at least 90%, at least 95%, or 100% sequence identity to SEQ ID NO: 119;
(aw) a polypeptide having at least 80%, e.g., at least 85%, at least 90%, at least 95%, or 100% sequence identity to SEQ ID NO: 120;
(ax) a polypeptide having at least 80%, e.g., at least 85%, at least 90%, at least 95%, or 100% sequence identity to SEQ ID NO: 121;
(ay) a polypeptide having at least 80%, e.g., at least 85%, at least 90%, at least 95%, or 100% sequence identity to SEQ ID NO: 122;
(az) a polypeptide having at least 80%, e.g., at least 85%, at least 90%, at least 95%, or 100% sequence identity to SEQ ID NO: 123;
(ba) a polypeptide having at least 80%, e.g., at least 85%, at least 90%, at least 95%, or 100% sequence identity to SEQ ID NO: 124;
(bb) a polypeptide having at least 80%, e.g., at least 85%, at least 90%, at least 95%, or 100% sequence identity to SEQ ID NO: 125;
(bc) a polypeptide having at least 80%, e.g., at least 85%, at least 90%, at least 95%, or 100% sequence identity to SEQ ID NO: 126;
(bd) a variant of SEQ ID NO: 72, SEQ ID NO: 73, SEQ ID NO: 74, SEQ ID NO: 75, SEQ ID NO: 76, SEQ ID NO: 77, SEQ ID NO: 78, SEQ ID NO: 79, SEQ ID NO: 80, SEQ ID NO: 81, SEQ ID NO: 82, SEQ ID NO: 83, SEQ ID NO: 84, SEQ ID NO: 85, SEQ ID NO: 86, SEQ ID NO: 87, SEQ ID NO: 88, SEQ ID NO: 89, SEQ ID NO: 90, SEQ ID NO: 91, SEQ ID NO: 92, SEQ ID NO: 93, SEQ ID NO: 94, SEQ ID NO: 95, SEQ ID NO: 96, SEQ ID NO: 97, SEQ ID NO: 98, SEQ ID NO: 99, SEQ ID NO: 100, SEQ ID NO: 101, SEQ ID NO: 102, SEQ ID NO: 103, SEQ ID NO: 104, SEQ ID NO: 105, SEQ ID NO: 106, SEQ ID NO: 107, SEQ ID NO: 108, SEQ ID NO: 109, SEQ ID NO: 110, SEQ ID NO: 111, SEQ ID NO: 112, SEQ ID NO: 113, SEQ ID NO: 114, SEQ ID NO: 115, SEQ ID NO: 116, SEQ ID NO: 117, SEQ ID NO: 118, SEQ ID NO: 119, SEQ ID NO: 120, SEQ ID NO: 121, SEQ ID NO: 122, SEQ ID NO: 123, SEQ ID NO: 124, SEQ ID NO: 125 or SEQ ID NO: 126 comprising one or more amino acid substitutions (preferably conservative substitutions), and/or one or more amino acid deletions, and/or one or more amino acid insertions or any combination thereof in 1, 2, 3, 4, 5, 6, 7, 8, 9 or 10 positions;
(be) a polypeptide comprising the polypeptide of (a), (b), (c), (d), (e), (f), (g), (h), (i), (j), (k), (1), (m), (n), (o), (p), (q), (r), (s), (t), (u), (v), (w), (x), (y), (z), (aa), (ab), (ac), (ad), (ae), (af), (ag), (ah), (ai), (aj), (ak), (al), (am), (an), (ao), (ap), (aq), (ar), (as), (at), (au), (av), (aw), (ax), (ay), (az), (ba), (bb), (bc) or (bd) and a N-terminal and/or C-terminal extension of between 1 and 10 amino acids; and
(bf) a fragment of the polypeptide of (a), (b), (c), (d), (e), (f), (g), (h), (i), (j), (k), (l), (m), (n), (o), (p), (q), (r), (s), (t), (u), (v), (w), (x), (y), (z), (aa), (ab), (ac), (ad), (ae), (af), (ag), (ah), (ai), (aj), (ak), (al), (am), (an), (ao), (ap), (aq), (ar), (as), (at),
(au), (av), (aw), (ax), (ay), (az), (ba), (bb), (bc) or (bd) having xylanase activity and having at least 90% of the length of the mature polypeptide.

7. The animal feed of claim 1, wherein the protein source is selected from the group consisting of soybean, wild soybean, beans, lupin, tepary bean, scarlet runner bean, slimjim bean, lima bean, French bean, Broad bean (fava bean), chickpea, lentil, peanut, Spanish peanut, canola, sunflower seed, cotton seed, rapeseed (oilseed rape) or pea or in a processed form such as soybean meal, full fat soy bean meal, soy protein concentrate (SPC), fermented soybean meal (FSBM), sunflower meal, cotton seed meal, rapeseed meal, fish meal, bone meal, feather meal, whey or any combination thereof.

8. The animal feed of claim 1, wherein the energy source is selected from the group consisting of maize, corn, sorghum, barley, wheat, oats, rice, triticale, rye, beet, sugar beet, spinach, potato, cassava, quinoa, cabbage, switchgrass, millet, pearl millet, foxtail millet or in a processed form such as milled corn, milled maize, potato starch, cassava starch, milled sorghum, milled switchgrass, milled millet, milled foxtail millet, milled pearl millet, or any combination thereof.

9. The animal feed of claim 1, wherein the animal feed additive further comprises one or more components selected from the list consisting of:
one or more additional enzymes;
one or more microbes;
one or more vitamins;
one or more minerals;
one or more amino acids; and
one or more other feed ingredients.

10. The animal feed of claim 1, wherein:
(a) the polypeptide having xylanase activity is formulated as a granule;
(b) the polypeptide having muramidase activity is formulated as a granule; or
(c) both the polypeptide having xylanase activity and the polypeptide having muramidase activity are formulated as granules.

11. The animal feed of claim 1, wherein
(a) the polypeptide having xylanase activity is formulated as a liquid;
(b) the polypeptide having muramidase activity is formulated as a liquid; or
(c) both the polypeptide having xylanase activity and the polypeptide having muramidase activity are formulated as liquids.

12. A method of improving growth performance of an animal comprising administering to the animal an animal feed according to claim 1.

13. A method of improving Body Weight Gain (BWG), Feed Conversion Ratio (FCR) and/or European Production Efficiency Factor (EPEF) of an animal comprising administering to the animal the animal feed according to claim 1.

14. Use of an animal feed according to claim 1 for improving growth performance of an animal.

15. Use of an animal feed according to claim 1 for improving Body Weight Gain (BWG), Feed Conversion Ratio (FCR) and/or European Production Efficiency Factor (EPEF) of an animal.


SEQUENCE LISTING (126 sequences)

SEQ ID NO: 1 - protein, 208 aa, Acremonium alcalophilum
RIPGFDISGWQPTTDFARAYANGDRFVYIKATEGTTFKSSAFSRQYTGATQNGFIRGAYHFAQPAASSGAAQARYFASNGGGWSKDGITLPGALDIEYNPNGATCYGLSQSAMVNWIEDFVTTYHGITSRWPVIYTTTDWWTQCTGNSNRFANRCPLWIARYASSVGTLPNGWGFYTFWQYNDKYPQGGDSNWFNGDASRLRALANGD

SEQ ID NO: 2 - protein, 213 aa, Acremonium alcalophilum
SPIRRRIPGFDISGWQPTTDFARAYANGDRFVYIKATEGTTFKSSAFSRQYTGATQNGFIRGAYHFAQPAASSGAAQARYFASNGGGWSKDGITLPGALDIEYNPNGATCYGLSQSAMVNWIEDFVTTYHGITSRWPVIYTTTDWWTQCTGNSNRFANRCPLWIARYASSVGTLPNGWGFYTFWQYNDKYPQGGDSNWFNGDASRLRALANGD

SEQ ID NO: 3 - protein, 218 aa, Aspergillus fumigatus
LPSQPEARATTVQGFDISNHQKSVNFEAAKKDGAQFVMIKATEGTTYKDTVFNSHYTGATKAGLLRGGYHFARPDKSTGSTQAKFFLKNGGGWSDDNRTLPGMLDIEYNPYGATCYGLSHSQMVAWIHDFVNEYHHATSRWPMIYTTADWWNRCTGNAKGFGDKCPLVLAAYSSSPPKTIPGDWKTWTIWQNSDKYKHGGDSDKFNGPMTQLRKLASG

SEQ ID NO: 4 - protein, 208 aa, Trichoderma reesei
TVPGFDISHYQATVDFAKAYADGARFVIIKATEGTTYTDPSFSDHYTKATNAGFIRGGYHFAQPASSSGAAQANYFLKHGGGWSADGITLPGMLDLEYAPSGDSCYGLSASAMVSWINDFVNTYHAATTQYPLIYTSTSWWQLCTGNNGSFGSKSPLVIARYASSVGALPNGWSVYTIWQNSDASPWGGDNDIFNGNLAQLQKIARGS

SEQ ID NO: 5 - protein, 215 aa, Trametes cinnabarina
SPTPEKRANPKGIDVSAYQPNINWSTVKANGISFAYIKATEGTTYTNPDFSSQYTGATNAGLIRGGYHFAHPDSSSGATQAKYFLAHGGGWTSDGITLPGALDIEYNPSGAECYGLSASAMVSWIKDFSNTYHSSTGVYPVIYTTTDWWTTCTGNSAAFASTNPLWIARYASSIGTLPAGWSYTTFWQYADSGPNPGDQDEFNGSMAGLKQLALG

SEQ ID NO: 6 - protein, 207 aa, Sporormia fimetaria
AVPGFDISHYQSSVNYAGAYSSGARFVIIKATEGTTYIDPKFSDHYIGATNAGLIRGAYHFARPAASTGAAQANYFVSHGGGWSADGITLPGMLDMEYGSTSACHGLSQSAMVTWITSFVNQYNSLTGRYPMIYTTADWWQTCTGNSAAFNTKSPLVLARYSSSAGTVPGGWPYYTIWQFNDAYAYGGDSDTFNGDLAGLKRLAKGS

SEQ ID NO: 7 - protein, 201 aa, Poronia punctata
AVQGFDISHYQSSVNFGAAKSSGAQFVIIKATEGTSYTDPSFSSHYTGATNAGLIRGGYHFAHLDSSSGAAQAKYFLAHGGGWSGDGITLPGMLDLEGSCVLSASATVSWIKDFSNTYHSSTGVYPLIYTNPSWWSSCTGNSKAFIDTNPLVLARYASSAGTPPGGWPYYTIWQYNDAYKYGGDSDVFNGDLAGLKRLAKG

SEQ ID NO: 8 - protein, 201 aa, Poronia punctata
QVQGFDISSYQPSVDFAGAYADGARFVIIKATEGTGYIDPTFSDHYVGATNAGLLRGGYHYAHLDSTSGATQAQYFLANGGGWSGDGITLPGMLDLEGDCVLSAADAVAWIKDFSDTYHASTGVYPLLYTNPSWWASCTGDSSAFIDTNPLVLAHYADAAGTPPGGWPFYSFWQYNDAYPYGGDSEVWNGDMDGLLRLASG

SEQ ID NO: 9 - protein, 203 aa, Lecanicillium sp. WMM742
VDSSSEVSVAIYKKALGQGFTRAIFRGYQEACSQGGRVDPTFVPSYKNAVAAGYKDFDAYFFPCTGKTNKCKPYAAQLAELLDTIKGQKLAIRRIWLDIETDRVCNPFDYGAQGNLAEAKKLVAAFRDAKLDWGIYTSPTQWETIFGAKTVELAKDVPLWFAKFDNVETLELKTPFGGWTKADAKQYTDQSASNKFDLNVFSA

SEQ ID NO: 10 - protein, 208 aa, Lecanicillium sp. WMM742
TVQGFDVSGYQPTVNWGAAYSSGARFVMIKATEGTGYISSSFGSQYPGATNAGFIRGGYHFALPDRSSGSAQADYFLAHGGGWSGDGITLPGMLDIEYNPYGATCYGLSQGAMVNWISDFVEHYKARTTQYPIIYTTTDWWKTCTGNSPAFGQKCPLSLARYSSSVGEIPNGWPFQTFWQNSDKYAYGGDSQIFNGAYSQLQKIARGG

SEQ ID NO: 11 - protein, 207 aa, Onygena equina
AVPGIDVSGYQGNVNWANVANAGKKFAYVKATEHTNYINPYFAQQYNGAYNQGIIRGAYHYAHPNGASGASQANYFLAHGGGWSADGKTLPGAVDLEYGPNGSTCWGISQSAMIAWIRDFSNTYRAKTGRPPVIYTSTSWWKTCTGNYGGFGNDNPLWIARYSSTVGELPAGWPFHSIWQNNDNSGVGGDGDIWNGDLAGLQRFAKG

SEQ ID NO: 12 - protein, 208 aa, Purpureocillium lilacinum
AVKGFDISHYQPNVDFAKAYADGARFVMIKATEGTTYTDPSFSSHYTGATKAGFIRGGYHFARPASSSGAAQAKYFIAHGGGWSKDGITLPGMLDMEYQSSSSACGGLSQSAMVSWINDFVNTYHAATGVYPLIYTSTSWWTQCTGNSAAFGSKCPLVVARYASSVGTLPAGWGFYTFWQYSDAAPWGGDADTFNGDITALKKIANAG

SEQ ID NO: 13 - protein, 207 aa, Trichobolus zukalii
AVPGFDISHYQPSVNYAGAYNSGARFVIIKATEGTTYTDPVFSTHYTGATKAGLIRGGYHFARPASSSGSAQADFFFKNGGGWSADGITLPGMLDMEYGSTSSCHGLSQTAMVNWISDFVNRYKTLSGRYPMIYTGYYWWVECTGNSNKFATTCPLVLARYSSSVGEIPGGWGYQTIWQFNDKYAYGGDSDSFNGSLDRLKALAKGT

SEQ ID NO: 14 - protein, 207 aa, Penicillium citrinum
LIHAVDSSSEVSVDIYKKALSEGFSRAIFRGYQEACSQGGRVDPTFLPSYKNAQTAGYKDFDAYFFPCTGSGNKCKPYDVQIGELVDAIKKNNMAIRRIWVDFEKDKTCNPFNWDPKRNIDEAKRIIGAVRKTKFDFGVYTSATQWTSIFGSKDVVLANDVPLWFAKFDNVENLDLAQPFGGWTKADGKQYTDKSASKKFDLNVFSA

SEQ ID NO: 15 - protein, 207 aa, Cladorrhinum bulbillosum
AVQGFDISHYQSSVNFQAAYNSGARFVIIKATEGTTYIDPKFSSHYTGATNAGLIRGGYHFAHPDSSTGAAQADFFLAHGGGWSGDGITLPGMLDLESVSGKATCFGLSASSMVAWIKSFSDRYHTRTGRYPMLYTNPSWWTTCTGNSNAFVNTNPLVLARYASAPGTIPGGWPYQTIWQNSDSYTYGGDSDIFNGALSGLQKLASG

SEQ ID NO: 16 - protein, 208 aa, Umbelopsis westeae
KLKGLDVSGYQPNVAWSTVKANGASFAYIKATEGTNYKNPSFAQQYNGAYNAGLIRGSYHFAQPSSSTGAAQANYFLAHGGGWSPDGKTLPGALDMEYNPHGSTCYGLSKDAMVKWIKDFSNTYHSATGRYPVIYTTTSWWTTCTGNSAAFGATNPLWIARYSSTAGNLPNGWAFYSFWQNADSGIFPGDQDIWNGDAAALSRMAKGA

SEQ ID NO: 17 - protein, 208 aa, Zygomycetes sp. XZ2655
TLPGLDVSSYQGNVNWGTVASQGAKFAYVKATEGTTYTNPYFASQYDGSYNAGLIRGAYHFAHPDSSSGATQANYFLAHGGGWSADGKTLPGALDIEYNPNGAECYGLSQLAMISWIQDFSNTYHSHTGRYPVIYTTTDWWTTCTGNSAAFGTNNPLWIARYSSSVGTLPAGWGYESFWQKASSGTFPGDQDIWNGDAAGLSRFATGK

SEQ ID NO: 18 - protein, 206 aa, Chaetomium cupreum
TVQGFDISGYQPNVNFAAAYAAGARFVIIKATEGTSYISPSFSSQYTGATNAGFIRGGYHFAHPGASSGTTQADYFIAHGGGWTPDGITLPGMLDLESESSGTCWGLSASAMVAWIKDFSDHYHSRMGVYPLLYTNPSWWEECTGNSNAFVDTNPLVLAHYSSSVGTIPGGWPYETIWQNSDSYAYGGDSDVFNGDLAGLQRLARG

SEQ ID NO: 19 - protein, 207 aa, Cordyceps cardinalis
LIHAVDSSSEVSVDVYKKALAEGFTRAIFRGYQEACSQGGRVDPTFLPSYKNAQKAGYKDFDAYFFPCTGSGNKCKPYAKQIGELVDAIEGNQLAIRRIWIDIETDKVCNAFNWGAEGNIQEAKKLIAAVRGTKRDFGIYTSATQWENIFGSRTVELAKDVPLWFAKFDNVETLELKTPFGGWTKADAKQYTDKSASKKFDLNVFSA

SEQ ID NO: 20 - protein, 216 aa, Penicillium sp. ′qii′
STIQPRASGVQGFDISSYQGTVNFAGAYGAGARFVMIKATEGTTYIDSTFSSHYDGATSAGLIRGAYHFAHPDSSSGATQAEYFLAHGGGWTNDGITLPGMLDIEYNPSGSTCYGLSASAMVSWIKDFGETYNSKTGRYPMIYSTADWWSTCTGDSTSFSSDYPLVLAQYASSISTVPGGWPYQSFWQNADSYSYGGDSDLWNGSEDSLKTFAKGS

SEQ ID NO: 21 - protein, 218 aa, Aspergillus sp. nov XZ2609
LPTKLAARYSTVQGFDVSNYQPNVDFSAAKSAGAEFVIIKATEGTDYKDTYFNSHYTGATNAGLIRGGYHFARPDKSSGTAQAEYFLAHGGGWSKDGRTLPGMLDIEYNPYGATCYGLSHSAMVSWVNEFLNEYHSKTGVYPLLYTTADWWNQCTGNAHGFGNKSPLVLASYSSESPRTVPGDWQTWTIWQNADKYKYGGDSDIFNGDLTQLKKIVEG

SEQ ID NO: 22 - protein, 204 aa, Paecilomyces sp. XZ2658
TVAGFDISNYQPSVNFAKAYADGARFATEGTTYIDPSFSSHYTGATNAGLIRGGYHFAHPGSSTGAAQATYFLAHGGGWSKDGITLPGMIDLEYNPSGATCYGLSTSAMVSWISDFVETYHSKTGVYPLIYTSTSWWNQCTGSSTAFASKCPLVVARYASSVGTLPAGWGYQTIWQNSDSSPWGGDNDIFNGSLDQLKRIANAS

SEQ ID NO: 23 - protein, 203 aa, Paecilomyces sp. XZ2658
AVQGHDVSHWQGNINWGAVKAAGVKFTYIKATESTNYIDPSFNANYVGATNTGLIRGAYHFARPGDSSGAAQANYFVSHGGGWSADGRTLPGALDLEAGCSGLSQSAMTAWIRDFSNTYHARTGRFPVIYTTTSWWKTCTGNASGFQNDHPLWIARWGPSPGELPAGYGFHTFWQYADKGPLPGDQDNFNGDEAGLARLARGS

SEQ ID NO: 24 - protein, 208 aa, Pycnidiophora cf dispera
AVSGMDISHYQGTNYNFAGAYSSGARFVIIKATEGTTYTDPQFSANYIGATNAGFIRGAYHFARPAASTGAVQASYFVSHGGGWSSDGITLPGMLDMEYGSTSTCHGLSVSAMNTWIASFVNQYKSLTGAYPMIYTTADWWKTCTGDSTAWNTKCPLVLARYSSSVGTIPGGWPYHTIWQYSDSYAYGGDSDTFNGDLAGLKRLAKGS

SEQ ID NO: 25 - protein, 210 aa, Thermomucor indicae-seudaticae
YQTGLDVSALTSTSSFSCAKNLGYDHVIARCYMEAYGNNPGGKVDPNCYSNYKNAKAAGFTSVDIYMFPCTGRSTCKSPATQVQEIVDYVGAHKMIVGTLWLDVEVDSAANNWPSTSEARSTLRAFKTALDKSGWKWGVYSSKSQWTRITGSASWVLDPSVPLWYSHYDDTLSFSDYPSHAFGGWSKPTIKQYTGDASFCSASWDKNYYG

SEQ ID NO: 26 - protein, 207 aa, Isaria farinosa
LTHAVDSSSEVSVDIYKKALGQGFTRAIFRGYQEACSLGGRVDPTFVPSYKNAVAAGYKDFDAYFFPCTGTTNKCKPYATQLAELLDTISSQKLAIRRIWLDIETDQVCSPFDYGAQGNIAEAKKLVAAFRAAKHDWGIYTSPTQWETIFGSKTFVLANDVPLWFAKFDNVETLDLKTPFGGWTKADAKQYTDQSASKKFDLNVFSA

SEQ ID NO: 27 - protein, 207 aa, Lecanicillium sp. WMM742
SVQGFDISHYQSSVNFGAAYADGARFVIIKATEGTTYRDPKFSEHYGGATKAGFIRGGYHFAQPASSSGAAQANFFLAHGGGWSGDGITLPGMLDLEYGPNGNTCYGLGPASMRSWISDFVETYHAKTGRYPLIYTSTSWWKTCTGNTSLFADKCPLVVARYNSQVGELPAGWGFYTFWQFNDHYKHGGDSDVFNGAYSQLQKIATG

SEQ ID NO: 28 - protein, 208 aa, Zopfiella sp. t180-6
AVQGFDVSHWQSSVNFAAAYNSGARFVIIKATESNNYIDPKFNTYYPAATSAGLIRGGYHFAHPGETTGAVQADYFIAHGGGWSSDGITLPGMLDLENASGYPACWGLSQSAMVSWIKAFSDRYKARTGVYPMLYTNPSWWTSCTGNSNAFVNTNPLVLARYASSPGTIPGGWPYQTIWQNSDSYTYGGDSDIFNGDLAGLKRLAKGS

SEQ ID NO: 29 - protein, 217 aa, Malbranchea flava
GPKEFESRASGVQGFDISGWQSNVNFAGAYNSGARFVMIKASEGTTFKDRQFSNHYIGATKAGFIRGGYHFALPDVSSATAQVNHFLASGGGWSRDGITLPGMLDIESNPYGAQCYGLDAGRMVAWIREFVDAYKRATGRYPLIYTSPSWWQTCTGNSNAFIDKCPLVLARWASSPGTPPGGWPFHSFWQYADSYQFGGDAQVFNGDEAGLKRMALG

SEQ ID NO: 30 - protein, 208 aa, Hypholoma polytrichi
LVYGVDSSSLVPVATYQKALGEGFTKAVIRGYEEACGVGGEVDPNFVPSYKNARAAGYTDIDMYWFPCNGSTHSCKSYAAQLAAIAAAFSANAMKIGTIWIDIEKDAAICNNWDYGTAGNLAQAKALIAAAKASGFNFGIYSSPGEWSTIFGSTSVVVDNSAPLWFATYNNVQTLTLGTPFGGWSTAVGHQYTDVSASGLFDLNVFAH

SEQ ID NO: 31 - protein, 201 aa, Aspergillus deflectus
AVQGFDISHYQSSVDYAGAYSSGARFVMIKATEGTTYTDPAFSTHYTGATNAGLIRGGYHFARPGSSSGAAQAEYFLAHGGGWTGDGITLPGMLDLEAGCSGLSASAMVSWIQDFGETYKASTGRYPMIYTTTSWWSSCTGNNGGFGDYPLVLARWASSPGELPNGWSVHSFWQNADTYEYGGDSEIWNGSQENLVKFASQ

SEQ ID NO: 32 - protein, 202 aa, Ascobolus stictoideus
AVPGFDISHYQSTVNFADAYSSGARFVIIKATEGTTYKDPKFSSHYTGATNAGLIRGGYHFARPASSTGAVQAQYFVSNGGGWSGDGLTLPGMLDLEGDCAGLSQAGMVSWITSFVNKYKALTTRYPMIYTTNSWWNTCTGNSQAFSANCPLVIARYNSVVGTIPGGWPYYTIWQFNDAYSYGGDSDTFNGAYSQLVKLATG

SEQ ID NO: 33 - protein, 207 aa, Coniochaeta sp
TVQGFDISHYQPTVNYAGAYNAGARFVIIKATEGTTYTDPSFSTHYNGATKAGLIRGGYHFAHPGVTTGAAEANFFLAHGGGWSGDGITLPGMLDLESEGSNPQCWGLSTSGMVAWIKSFSDRYHTVTGRYPMLYTNPSWWSTCTGNSNAFVNTNPLVLARYASAPGTIPGGWPYQTIWQNSDSYSYGGDSDIFNGNLASLQKLATG

SEQ ID NO: 34 - protein, 202 aa, Daldinia fissa
AVPGFDISHYQSSVDFGAAYSSGARFVIIKATEGTTYQDPKFSSHYAGATNAGLIRGGYHFARPASSSGAAQATFFLAHGGGWSGDGITLPGMLDLEGDCAGLSTSAMVSWIRDFSDTYHGKTGRYPLLYTNPSWWSSCTGGSSAFVNTNPLVLARYASSPGALPGGWPYYTIWQFNDAYKYGGDSDTFNGDLTQLKKLASG

SEQ ID NO: 35 - protein, 201 aa, Rosellinia sp
AVQGFDISHYQSSVNFAGAYSAGARFVIIKATEGTSYIDPKFSSHYIGATNAGLIRGGYHFAHLGSSSGAAQANYFLAHGGGWSGDGITLPGMLDLEGDCVLSASGAVAWIKDFSDTYHSKTGVYPLLYTNPSWWSSCTGNSNAFVNTNPLVLARYSSSAGTPPGGWPYYTIWQYNDAYAYGGDSDVFNGDMAGLLRLAKG

SEQ ID NO: 36 - protein, 202 aa, Ascobolus sp. ZY179
AVPGFDISHWQSSVNFASAYSSGARFVIIKATEGTTYKDPKFSSHYTGATKAGFIRGGYHFARPASSTGAAQAQFFASNGGGWSGDGITLPGMLDLEGDCAGLSQSGMVSWISSFVNKYRSLTGRYPMIYTTNSWWVTCTGNSKAFSSNCPLVIARYNSVVGTIPGGWPYYTIWQYNDAYKYGGDSDTFNGAYSQLVKLATG

SEQ ID NO: 37 - protein, 206 aa, Curreya sp. XZ2623
TVPGFDISHYQGTVNFAGAYSSGARFVIIKATEGTTYTDPNFSNNYVGATNAKFIRGAYHFARPDGGSGSTQAQFFHSHGGGWSGDGITLPGMLDIEYGPTSTCYGLSTSAMVTWITDFVNEYHALTGRYPLIYTTNDWWNTCTGNTNKFSTTCPLVLARYSSSVGTIPGGWPFQTIWQFNDNYAYGGDSDTFNGDLAGLKKLATG

SEQ ID NO: 38 - protein, 202 aa, Coniothyrium sp
AVQGFDISHYQASVNFAAAYSGGLRFVYIKATEGTTYQDPAFSSHYSGATSAGFIRGGYHFARPASSTGAAQASYFVAHGGGWSNDGITLPGMLDLEGDCAGLSTASMVSWISSFSNQYHSLTGRWPVIYTTNSWWTTCTGNSAAFNANSPLMLARWGSTAGTIPGGWPYYTIWQYKDSNTYGGDSDVFNGDATQLKKLATG

SEQ ID NO: 39 - protein, 202 aa, Hypoxylon sp
AVPGFDISHYQSNVDFGAAYSSGARFVIIKATEGTTYQDPSFSTHYTGATKAGLIRGSYHFARPGSSSGAAQATYFLAHGGGWSGDGITLPGMLDLEGDCAGLSTNAMVAWIRDFSDTYHGRTGRYPLLYTNPSWWSGCAGGSAAFVGTNPLVLARYAGSPGALPGGWPYYTIWQFDDAYKYGGDSDTFNGDLTQLKKLASG

SEQ ID NO: 40 - protein, 202 aa, Xylariaceae sp. 1653h
AVPGFDISHYQSSVDFAAAYSAGARFVIIKATEGTTYIDPSFSSHYTGATNAGLIRGGYHFAHPGSSSGATQANYFLAHGGGWSGDGITLPGMLDLEGDCAGLSTSAMVSWIKDFSNAYHSKTGRYPLLYTNPSWWSSCTGSSSAFVNTNPLVLARYSSSAGTPPGGWPYYTIWQFNDAYKYGGDSDTFNGEYASLQKLATG

SEQ ID NO: 41 - protein, 202 aa, Hypoxylon sp
AVPGFDISHYQSSVDFAAAYNSGARFVIIKATEGTTYIDPSFSSHYTGATKAGFIRGGYHFAHPGSSSGAAQANYFLAHGGGWSGDGITFPGMLDLEGDCAGLSTSAMVSWIKDFSDTYHSKTGRYPLLYTNPSWWSSCTGDSSAFVNTNPLVLARYSSSAGTPPGGWPYYTIWQFNDAYKYGGDSDTFNGNYASLQKLATG

SEQ ID NO: 42 - protein, 206 aa, Yunnania penicillata
DVDGFDISHYQETVDYAGAYGAGARFVIIKATEGTNYIDSSFNTHYAGATDAGLIRGGYHFAHPGETTGAEQADYFIAHGGNWSNDGITLPGMLDLEGEGSTTCWDLSAADMVAWIKAFSDRYQEVTSRYPLLYTNPSWWSECTGNSDAFVDTNPLVLARYASSPGEIPGGWPAQTIWQNSDSYSFGGDSDIFNGDEAGLKKLASG

SEQ ID NO: 43 - protein, 207 aa, Engyodontium album
RVQGFDISHYQPSVDFNAAYADGARFVIIKATEGTTYKDPKFSQHYIGATNAGFIRGGYHFAQPASSSGAAQADYFLKNGGGWSSDGITLPGMLDMEYNPNGSACYGLSQASMRNWINDFVNTYHSRTGVYPLLYTTTSWWKTCTGNTAMFADKCPLVIARYNSVVGELPAGWSFWTIWQYNDHYKHGGDSDAFNGDYSQLQRIARG

SEQ ID NO: 44 - protein, 208 aa, Metapochonia bulbillosa
TVAGFDISNYQPTVDFKKAYADGARFVIIKATEGTTYTDPSFSSHYTGATQAGLIRGGYHFAHPGSGTGAAQANYFLAHGGGWSKDGITLPGMIDLEYNPSGATCYGLSASGMVSWISDFVETYHSKTGVYPLIYTSTSWWNQCTGSSTAFGNKCPLVVARYASSVGALPAGWGFQTIWQNSDKSPWGGDNDIFNGSLDQLKRIANAS

SEQ ID NO: 45 - protein, 215 aa, Hamigera paravellanea
APLEARAGSVQGFDISHYQAKVDFAAAYRSGARFVIIKATEGTTYTDPAFSSHYTSATNAGFIRGGYHFAHPDSSSGAAQATYFLAHGGGWSGDGITLPGMLDLEYNPSGATCYGLSDAAMVAWIQDFVDTYHARTGRYPMIYTTADWWNTCTGNSSKFSQTCPLVLARYASSVGTVPGGWGYQTIWQNSDSYAYGGDSDIFNGDETQLKKLASG

SEQ ID NO: 46 - protein, 217 aa, Metarhizium iadini
SPVELEQRAASVKGFDISGYQPNVDFNKAYADGARFVIIKATEGTTYIDKTFSKHYTGATKAKLIRGAYHFAHPGQNKASAEADFFVQHGGNWSKDAITLPGMVDLESEKGHPPCWGLSHSAMVAWISEFVAAYHKKTTRYPMLYTNPSWWSACTGNSKAFKDTCPLVLARYASSPGAIPGGWPAQTIWQNSDKSPWGGDSDMFNGDLAQLKKLATG

SEQ ID NO: 47 - protein, 214 aa, Thermoascus aurantiacus
ELDKRARGVQGFDISHYQPNVDFKGAYNSGARFVIIKATEGTTYKDPAFSKHYIGATEAGLIRGGYHFAHPDKSSGAAQANFFLAHGGGWSGDGITLPGMVDLEYNPSGDACYGLSDSQMVSWIRDFVNTYHAHTGRYPMIYTTADWWKRCTGDSHAFSTTCPLVLARYNSSPGTVPGGWPYHTIWQNSDKYRFGGDSDIFNGDLAGLKRLAKG

SEQ ID NO: 48 - protein, 208 aa, Clonostachys rossmaniae
AVPGFDISGWQKSTDFAKSYANGDRFVYIKATEGTTFKNPLFSKQYTGATNARLIRGAYHFAQPASSSGASQARFFVANGGGWSNDGITLPGAVDMEYNPSGATCYGLSKTAMVNWIEDFVSTYQALTGRWPVVYTTLDWWTQCTGNSAKFGDRCPLWVARYASAVGQIPAGWSFHTIWQYNAKYPEGGDSDIFNGDETRLKALASGA

SEQ ID NO: 49 - protein, 203 aa, Simplicillium obclavatum
APKGIDVSHWQGSINWGAVKANGIEWAYIKATESTNYKDPNFNANYVGATNAGLIRGAYHFARPGDSSGAAQANYFASNGGGWSADGITLPGAVDLEAGCSGLSQSAMTAWIKDFSNTYHARTGRYPAIYTTTSWWKQCTGNASGFQNNNPLWIARWASSVGELPAGYSYHTFWQYADHGPNPGDQDVFNGDSAGLKRMAKGS

SEQ ID NO: 50 - protein, 216 aa, Aspergillus inflatus
APLEARANTVQGFDISSFQPNVDFAAAYKAGARFVMMKATQNTNYVDKTFNAHYEGATKAGLIRGGYHFAIPNGPSGAAQAEYFLAHGGGWSDDGKTLPGMIDLEYNPYGQTCYDLSAAKMVDWIKDFSNTYHAKTKRYPMIYTTANWWKECTGDSKEFSQTNPLVLARYSSSAGTVPGGWPAYSFWQNADKYKFGGDSDIWNGSEDNLKKFAKGA

SEQ ID NO: 51 - protein, 207 aa, Paracremonium inflatum
KVLGFDISHYQATVDFNAAKDAGARFVIIKATEGTTYKDPAFSKHYTGATKAGLIRGGYHFAQPASSSGAAQATFFLAHGGGWSSDGITLPGMLDLEYNPSGSTCYGLSQSSMVQWISDFIDTYHSKTGRYPLIYTSTSWWKTCTGNSSKFAANCPLVVARYSSSVGELPAGWTYYTIWQNSDSYKYGGDSDIFNGDESQLQKLAKG

SEQ ID NO: 52 - protein, 208 aa, Westerdykella sp
AVSGMDISHYQGTNYNFAGAYSSGARFVIIKATEGTTYTDPQFSANYIAATNAGFIRGGYHFARPADSTGAAQAKYFVSHGGGWSSDGITLPGMLDLEYGSSSACHGLSVSAMNTWIASFINQYRSLTGAYPMIYTTADWWKTCTGDSQAWNTKCPLVLARYSSSVGTIPGGWPYQTIWQFNDSYKYGGDSDTFNGDLAGLKRLAKGS

SEQ ID NO: 53 - protein, 207 aa, Stropharia semiglobata
LTYAVDSSTLVSVATYTKAKSQGFTKAIIRGYQEACGSGGAVDPNFVQTYKNARAAGYTDIDMYWFPCNGSTHNCKSYATQIAAIAATFSANSMKIGRIWIDIEKDAAVCNNWNYGTAGNLSQAKALISAIKASGFVYGIYSSPGEWGNIFGSTSVVVDNSAPLWFATWNNVQTLTMGTKFGGWTSAMGHQYTDVSASGQFDLSVFA

SEQ ID NO: 54 - protein, 208 aa, Gelasinospora cratophora
TVQGFDISHYQSSVNFAGAYSSGARFVIIKATEGTTYIDSSFSSHYTGATSAGLIRGGYHFAHPDSSTGAAQADYFLAHGGGWSADGITLPGMIDLESVSGKATCFGLSTSAMVSWIKSFSDRYYAKTGRYPMIYTNYSWWNQCTGNSASFAATNPLVLARWSSTVGTLPGGWSVQTIWQNADTYTYGGDSDVFNGSLDRLKALAKGS

SEQ ID NO: 55 - protein, 207 aa, Flammulina velutipes
RLNGIDVSGYQPNVNWATVKANGVSFAYIKATEGTTYTNPSFSSQYTGATKAGLIRGSYHFAHPSSSTGAAQARYFVAHGGGWSGDGITLPGALDIEYNPSGATCYGLSTSSMVNWIADFSNTYHSLTGRYPVIYTTADWWRTCTGNSASFANNSPLWIARYASTIGTLPAGWSYATFWQYADSGSNPGDQDYFNGDAAGLKRLATS

SEQ ID NO: 56 - protein, 207 aa, Deconica coprophila
LVHAVDSSSLVSTATFSKAKSEGFTKAVIRGYQEACGSGGRVDPNFVQTYKNARAAGITNIDTYWYPCNGSGNSCKSYAKQIAGISATFNAHSMKIGRIWIDIEKDSICNNWNYGTSGNRDHAKKLITAIKNSGFKYGIYSSPGEWSTIFGSESFDLDSGAPLWFATWNNVQTLTLGTHFGGWTSAHGHQYTDKSASGQFDLNVFSS

SEQ ID NO: 57 - protein, 208 aa, Rhizomucor pusillus
YETGVDVSALTSTSAWSCAKKLGYDHAIVRCYIEAYGGNPGGKIDSNCFQNYKNAKAGGFTSVDIYMFPCTGRSTCKSPAAQVKEVVDYVGSNKMTVGRLWLDVEIDPSANNWPSASSARSTLKSFKSALDSTGWKYGIYSSASQWSQITGSSSWELDSSLPLWYAHYDASLSFSDFSPFGGWTKPTIKQYAGSVSFCSAGWDKNYYG

SEQ ID NO: 58 - protein, 207 aa, Stropharia semiglobata
LVYGVDSSTLVSTATYSKAKSEGFTKAIIRGYQEACGSGGRVDPNFVATYKNARAAGITDIDMYWFPCNGSGNSCKSYAKQLSEIANVFSANSMKIGTIWIDFEKDSGCNNWNYGTTGNLNHAKALISAIKATGFKFGIYSSPGEWGTLFGSTGVVLDSSAPLWFATWNNVKTLTLGTHFGGWTKAVGHQYTDVSASGQFDLNVFAN

SEQ ID NO: 59 - protein, 207 aa, Stropharia semiglobata
LVYGVDSSTLVSTATYKKAKSEGFTKAIIRGYQEACGSGGRVDPNFVATYKNARAAGITDIDMYWFPCNGSGNSCKSYAKQLSEIANVFSANSMKIGTIWIDFEKDSGCNNWNYGTTGNLNHAKALISAIKATGFKFGIYSSPGEWGTLFGSTGVVLDSSAPLWFATWNNVKTLTLGTHFGGWTTAAGHQYTDVSSSGQFDLNVFAN

SEQ ID NO: 60 - protein, 207 aa, Myceliophthora fergusii
AVQGFDISHWQSSVDFKAAYNSGARFVIIKATEGTSFIDPKFSSHYTGATNAGFIRGAYHFAHPGQSSGEAQADYFLAHGGGWTPDGITLPGMLDLEAYNAGECWGLSQSAMVAWIKAFSDRYHARTGVYPMLYTNLSWWKTCTGNSKAFVNTNPLVLARWASSPGEIPGGWPWQTIWQNSDSYRYGGDSDIFNGDMNQLRRLATAA

SEQ ID NO: 61 - protein, 204 aa, Mortierella alpina
ALPKGIDVSHWQGDVNWNSVKAAGIEFVYIKATESINYIDSKFDANYVGATNAGLIRGGYHFARPAASSGAVQANYFLANGGGWSSDGITLPGALDLEAGCSGLSQAAMTAWVRDFSDTYHARTGRYPVIYTTTSWWKQCTGNASGFQNNNPLWIARWASSAGELPAGYAFHTFWQYADKGPNPGDQDYFNGDSAGLRRFAKGS

SEQ ID NO: 62 - protein, 216 aa, Penicillium atrovenetum
TPLESRASGVQGFDISSYQGTVDFAGAYAAGARFVMIKATEGTTYTDKTFSSHYEGASSAGLIRGGYHFAHPDSSSGAKQAEYFLAHGGGWSNDGKTLPGMLDIEYNPSGATCYGISKSAMVAWVKDFGETYKGKTGRYPMIYTTADWWNTCTGGSTAFSKDYPLVLARYSSSVGTIPGGWPYQSFWQNSDKYTYGGDSDLWNGSEASLKTFAKGA

SEQ ID NO: 63 - protein, 245 aa, Trichophaea saccata
YPVKTDLHCRSSPSTSASIVRTYSSGTEVQIQCQTTGTSVQGSNVWDKTQHGCYVADYYVKTGHSGIFTTKCGSSSGGGSCKPPPINAATVALIKEFEGFVPKPAPDPIGLPTVGYGHLCKTKGCKEVPYSFPLTQETATKLLQSDIKTFTSCVSNYVKDSVKLNDNQYGALASWAFNVGCGNVQTSSLIKRLNAGENPNTVAAQELPKWKYAGGKVMPGLVRRRNAEVALFKKPSSVQAHPPKC

SEQ ID NO: 64 - protein, 249 aa, Chaetomium thermophilum
PASAYAITGDNVNCRSGPGTSYAVKKVYKKGTDVKISCQTTGTNINGNNLWDKTSDGCYVSDYYVKTGSNGYVTSKCSSSGGSTCAAPKSNQATVDLIAEFEGFRANIYTDAAGYATVGYGHKCQKAKCAEVKYKIPLSKADGKKLLADDMRSFEVCITNMLNSKAKLNYNQFGALVSWSFNVGCGAAKSSTLIKRLNNGENVNKVLSEELPKWNKAGGKVLQGLVRRRAAEVALAKKSGSSQALPVKC

SEQ ID NO: 65 - protein, 248 aa, Trichoderma harzianum
YPITGDVVNCRTGPGTSYAIKKSYKKNQDISISCQTAGTSVNGNSIWDKTADGCYVADYYVKTGSSGYVTKKCTASSGGGSSSSYCKTINSAGVDLIAKWEGFVASPKPDPIGLPTVGYGHLCQQKNCREVKYKFPLTKTTAKELLLDDLPKYTKCLADYLNDKPKLNANQWAALTSWVFNVGCGNAKTSTLVKRLNNGEAANTVAAEELPKWRMAGGKVLPGLEARRKDEVKLFKTASSKQAYPKCQ

SEQ ID NO: 66 - protein, 245 aa, Trichophaea minuta
YPAKVDLRCRSSPSTSASVVRTYSKGSEIQISCQTTGTSVEGSNVWDKTQHGCYVADYYVKTGHSGIFTTKCGSSSGGGSCKPPPINAATVALIKEFEGFVAKPAPDPIGLPTVGYGHLCKTKGCKEVPYSFPLTQTTATKLLQSDIKTFTSCVSNYVKDSVKLNDNQFGALSSWAFNVGCGNIQTSSLIKRLNAGENPNTVAAQELPKWKYAGGKVLPGLVRRRKAEVALFKKPSSVQAHPPKC

SEQ ID NO: 67 - protein, 249 aa, Chaetomium sp. ZY287
YKISGSSVNCRSGPGTNYPVKKTYANGDEVTISCQTTGTNVEGNNIWDKTQHGCYVADKYVKTGKDGFVTKKCGSSGGGGGGKTCKAPKSNAATVDLIASFEGFRANIYTDATGHPTVGYGHMCTKSRCAEVKYKIPLSKADGKKLLADDMAKFEKCIKEMLNSKAKLNLNQYGALVSWSFNVGCGAAKGSQLVSRLNKGENPNTVLSNELPKWVHGNGKVLPGLVRRRNAEIALAKKSGSGAALPVKC

SEQ ID NO: 68 - protein, 245 aa, Mortierella sp. ZY002
YPITGADALHCRSGPGTSYPIQKTLRPPQDIKIQCQEPGTVVNGVSLWDKTQFGCYVSDYYVKTGTGNYVAPRCNSGGSSSACTGLNDAGINLIKEFEGFVPRPAPDPIGLPTVGYGHLCQTKGCGEVKYSFPLTTATATALLKDDLPKYTSCLAKALNGKPKLNKNQWAALASWTFNVGCGNMKSSSLITRLNAGQNPNTVATEELPKWKLAGGKVLPGLVRRRAAEVKLFKTANSSQGYPKCA

SEQ ID NO: 69 - protein, 247 aa, Metarhizium sp. XZ2431
YPVSADSLNCRAEPNTSSAIKTTYKKGEDVKISCQTEGPSINGNTIWDKTQDGCYVADYYVKTGSSGYVTGKCGGSSPPSGSGFCKTVNKAGLDLITKWEGFVSSPRGDPIGLPTVGYGHLCQKKGCAEVKYKFPLTKATALQLLNDDLPKYTGCLGKLLNSKVKLNDNQWAALTSWVFNVGCGNAQSSSLVRRLNNGENPNTVAPSELPKWKMAGGKVLEGLVKRRADEVRLFKVSSSKGAFPKCQ

SEQ ID NO: 70 - protein, 250 aa, Geomyces auratus
AFPITGSTVNCRTGPGTSHGVKTSYKKGHEVTVSCQTGGTSVNGNSIWDKTSDGCYVADYYVKTGSSGYVKPKCGSSSGGGGGSSCGAPKSNAATVNLIAEFEGFVSHVYTDATGHPTVGYGHLCSNSKCSGIGYSIPISKANAKKLLAKDMAIAEKCITAMINKSRTLNLNQYGALVSWAFNEGCGAAKSSTLIKRINNGEKPSTVIPQELPKWVYGGSSVLPGLVRRRNAEIALAKKATSSKALPAHC

SEQ ID NO: 71 - protein, 240 aa, Ilyonectria rufa
YKITGDNVNCRSGPGTSYSVKRSFKKGTDVTLSCQTTGENVLGTSIWDKTSYGCYVSDYYVKTGSSGFVVKKCGTCGAPKSNAATVNLISDFEGFRANIYKDAAGYPTVGYGHLCSNSRCTDVPYSIPLSKANGKNLLATDMTKFEKCITAMVSSSVTLNKNQYGALVSWAFNMGCGATKTSTLIKRLNQGQNVNTVLSTELPKWVYAGGKKLNGLVRRRNAEIALAKKKTTEKALPNKC

SEQ ID NO: 72 - protein, 384 aa, Aspergillus aculeatus
VGLDQAAVAKGLQYFGTATDNPELTDIPYVTQLNNTADFGQITPGNSMKWDATEPSQGTFTFTKGDVIADLAEGNGQYLRCHTLVWYNQLPSWVTSGTWTNATLTAALKNHITNVVSHYKGKCLHWDVVNEALNDDGTYRTNIFYTTIGEAYIPIAFAAAAAADPDAKLFYNDYNLEYGGAKAASARAIVQLVKNAGAKIDGVGLQAHFSVGTVPSTSSLVSVLQSFTALGVEVAYTEADVRILLPTTATTLAQQSSDFQALVQSCVQTTGCVGFTIWDWTDKYSWVPSTFSGYGAALPWDENLVKKPAYNGLLAGMGVTVTTTTTTTTATATGKTTTTTTGATSTGTTAAHWGQCGGLNWSGPTACATGYTCTYVNDYYSQCL

SEQ ID NO: 73 - protein, 288 aa, Clostridium acetobutylicum
AMSHSKFVGNIIAGSIPSNFDTYWNQVTPENATKWGAIEYGRGNYNWGSADLIYNYARSKNMPFKFHNLVWGSQQLTWLSNLSPQDQKSEVSKWIAAAGQRYSGSAFVDVVNEPLHTQPSYKNALGGDGSTGYDWIVWSYQQARKAFPNSKLLINEYGIIGDPNAAANYVKIINVLKSKGLIDGIGIQCHYFNMDNVSVGTMNYVLNMLSNTGLPIYVSELDMTGDDSTQLARYQQKFPVLYQNPNVKGITLWGYMQGQTWNSGTYLVNSNGTERPALKWLRSYLASH

SEQ ID NO: 74 - protein, 308 aa, Aspergillus aculeatus
NPIEPRQASVSIDAKFKAHGKKYLGTIGDQYTLNKNAKTPAIIKADFGQLTPENSMKWDATEPNRGQFSFSGSDYLVNFAQSNGKLIRGHTLVWHSQLPSWVQSISDKNTLIQVMQNHITTVMQRYKGKVYAWDVVNEIFNEDGSLCQSHFYNVIGEDYVRIAFETARAVDPNAKLYINDYNLDSASYPKLTGLVNHVKKWVAAGVPIDGIGSQTHLSAGAGAAVSGALNALAGAGTKEVAITELDIAGASSTDYVNVVKACLNQPKCVGITVWGSSDPDSWRSSSSPLLFDSNYNPKAAYTAIANAL

SEQ ID NO: 75 - protein, 328 aa, Thermotoga maritima MSB8
SQNVSLRELAEKLNIYIGFAAINNFWSLSDAEKYMEVARREFNILTPENQMKWDTIHPERDRYNFTPAEKHVEFAEENDMIVHGHTLVWHNQLPGWITGREWTKEELLNVLEDHIKTVVSHFKGRVKIWDVVNEAVSDSGTYRESVWYKTIGPEYIEKAFRWAKEADPDAILIYNDYSIEEINAKSNFVYNMIKELKEKGVPVDGIGFQMHIDYRGLNYDSFRRNLERFAKLGLQIYITEMDVRIPLSGSEEYYLKKQAEVCAKIFDICLDNPAVKAIQFWGFTDKYSWVPGFFKGYGKALLFDENYNPKPCYYAIKEVLEKKIEERK

SEQ ID NO: 76 - protein, 337 aa, Ascobolus stictoideus
NTVPSALEPRQAQTIQAALLAKGKKYFGTSITIRNDNTEQNLMRGNEFGSLTPENSMKWDAIQPNRNQFNWGGADQVANFATQNGKQLRCHTLVWHSQLPGWVSNSGFNNATLIQVMTTHIQTVMARYRGKCTHWDVVNEALEENGSWRNSVFYRVIGPAFIPIAFRIAAQADPSAILYYNDYNLEYRGPKHRAALEIVKLVKSYGVRIDGVGLQGHLVVERTPTQETPTPSLDVLQGVLEDYAALGVDTAYTEIDIRMNSPFTQQKLNTQAEHYARVAQSCMNVPRCVGLTVWGVSDRYSWVPGTFPGEGAALLWNDNYQKKPAYDAVLRAINNSS

SEQ ID NO: 77 - protein, 323 aa, Ustilago maydis
TLIPAKCKHEAFSQRAGSSLNAAIKSDGRKYFGTCADPGTLGNWQISNIIKAEMGQVTPENSMKWDATQPQRGTFNFGNADRLVDFATSNGKLIRGHTLVWHSQLPSWVSSITDANDLTNVIQNRIATVVGRYKGKVYAWDVVNEMFNENGSFRESVFYKLLGEDFVKIAFEAARKADPNAKLYINDYNLDDPDYPKLKSLVANVKKWRSQGVPIDGIGSQSHLQAAGHFLDASKVGGAMQALCAAASECAMTELDIAQASPDQYTKATEACLNQKNCVGITVWGVSDNTSWRKNANPLLWNSSYQKKPAYNAVLSTLNSYQA

SEQ ID NO: 78 - protein, 381 aa, Talaromyces emersonii
AGLNTAAKAVGLKYFGTATDNPELSDTAYETELNNTQDFGQLTPANSMKWDATEPQQNTFTFSGGDQIANLAKANGQMLRCHNLVWYNQLPSWVTGGSWTNETLLAAMKNHITNVVTHYKGQCYAWDVVNEALNDDGTYRSNVFYQYIGEATSPIAFATPPPPTPTPSCTTTTTTSSTPGVKATAAQNIVKLVQSYGARIDGVGLQSHFIVGQTPSTSAQQQNMAAFTALGVEVAITELDIRMQLPETSAQLTQQATDYQSTVQACVNTDSCVGITLWDWTDKYSWVPSTFSGWGDACPWDDNYQKKPAYNGILTALGGTPSSSTSYTLTPTTTPSGGSGSPTDVAQHWEQCGGLGLGLGPTVCASGFTCTVINEYYSQCL

SEQ ID NO: 79 - protein, 386 aa, Talaromyces emersonii
AGLNTAAKAIGLKYFGTATDNPELSDTAYETQLNNTQDFGQLTPANSMKWDATEPEQNVFTFSAGDQIANLAKANGQMLRCHNLVWYNQLPSWVTSGSWTNETLLAAMKNHITNVVTHYKGQCYAWDVVNEALNDDGTYRSNVFYQYIGEAYIPIAFATAAAADPNAKLYYNDYNIEYPGAKATAAQNLVKLVQSYGARIDGVGLQSHFIVGETPSTSSQQQNMAAFTALGVEVAITELDIRMQLPETEALLTQQATDYQSTVQACANTKGCVGITVWDWTDKYSWVPSTFSGYGDACPWDANYQKKPAYEGILTGLGQTVTSTTYIISPTTSVGTGTTTSSGGSGGTTGVAQHWEQCGGLGWTGPTVCASGYTCTVINEYYSQCL

SEQ ID NO: 80 - protein, 208 aa, Myceliophthora thermophila
RPFDFDDGNSTEALAKRQVTPNAQGYHSGYFYSWWSDGGGQATFTLLEGSHYQVNWRNTGNFVGGKGWNPGTGRTINYGGSFNPSGNGYLAVYGWTHNPLIEYYVVESYGTYNPGSQAQYKGSFQSDGGTYNIYVSTRYNAPSIEGTRTFQQYWSIRTSKRVGGSVTMQNHFNAWAQHGMPLGSHDYQIVATEGYQSSGSSDIYVQTH

SEQ ID NO: 81 - protein, 203 aa, Lasiodiplodia theobromae
APAAEAGELIARQSLTSSQTGTNNGYYYSFWTDGGGAVTYTNGAAGKYSVQWSGGNGNFVAGKGWSTGSARNIKFSGSFNPNGNGYLSVYGWTTSPLIEYYIVESYGDYNPGSEGTKKGTVTSDGSVYDIYTSTRTNAPSIQGTATFTQYWSIRRNHRSSGTVTTKNHFDAWAKLGLKLGTHNYQIVATEGYHSSGSSSITVS

SEQ ID NO: 82 - protein, 206 aa, Penicillium funiculosum
FPSELAQRAAGDLSKRQSITTSQTGTNNGYYYSFWTNGGGEVTYTNGDNGEYSVTWVDCGDFTSGKGWNPANAQTVTYSGEFNPSGNAYLAVYGWTTDPLVEYYILESYGTYNPSSGLTSLGQVTSDGGTYDIYSTQRVNQPSIEGTSTFNQYWSVRTEKRVGGTVTTANHFAAWKALGLEMGTYNYMIVSTEGYESSGSSTITVS

SEQ ID NO: 83 - protein, 185 aa, Bacillus subtilis
ASTDYWQNWTDGGGIVNAVNGSGGNYSVNWSNTGNFVVGKGWTTGSPFRTINYNAGVWAPNGNGYLTLYGWTRSPLIEYYVVDSWGTYRPTGTYKGTVKSDGGTYDIYTTTRYNAPSIDGDRTTFTQYWSVRQSKRPTGSNATITFSNHVNAWKSHGMNLGSNWAYQVMATEGYQSSGSSNVTVW

SEQ ID NO: 84 - protein, 190 aa, Trichoderma viride
QTIGPGTGFNNGYFYSYWNDGHGGVTYTNGPGGQFSVNWSNSGNFVGGKGWQPGTKNKVINFSGTYNPNGNSYLSVYGWSRNPLIEYYIVENFGTYNPSTGATKLGEVTSDGSVYDIYRTQRVNQPSIIGTSTFYQYWSVRRTHRSSGSVNTANHFNAWAQQGLTLGTMDYQIVAVEGYFSSGSASITVS

SEQ ID NO: 85 - protein, 220 aa, Thermopolyspora flexuosa
DTTITQNQTGYDNGYFYSFWTDAPGTVSMTLHSGGSYSTSWRNTGNFVAGKGWSTGGRRTVTYNASFNPSGNAYLTLYGWTRNPLVEYYIVESWGTYRPTGTYKGTVTTDGGTYDIYETWRYNAPSIEGTRTFQQFWSVRQQKRTSGTITIGNHFDAWARAGMNLGSHDYQIMATEGYQSSGSSTVSISEGGNPGNPGNPGNPGNPGNPGNPGGGCVATL

SEQ ID NO: 86 - protein, 204 aa, Trichoderma reesei
APAAEVESVAVEKRQTIQPGTGYNNGYFYSYWNDGHGGVTYTNGPGGQFSVNWSNSGNFVGGKGWQPGTKNKVINFSGSYNPNGNSYLSVYGWSRNPLIEYYIVENFGTYNPSTGATKLGEVTSDGSVYDIYRTQRVNQPSIIGTATFYQYWSVRRNHRSSGSVNTANHFNAWAQQGLTLGTMDYQIVAVEGYFSSGSASITVS

SEQ ID NO: 87 - protein, 210 aa, Trichoderma reesei
MPTGLEPESSVNVTERGMYDFVLGAHNDHRRRASINYDQNYQTGGQVSYSPSNTGFSVNWNTQDDFVVGVGWTTGSSAPINFGGSFSVNSGTGLLSVYGWSTNPLVEYYIMEDNHNYPAQGTVKGTVTSDGATYTIWENTRVNEPSIQGTATFNQYISVRNSPRTSGTVTVQNHFNAWASLGLHLGQMNYQVVAVEGWGGSGSASQSVSN

SEQ ID NO: 88 - protein, 185 aa, Bacillus subtilis
AGTDYWQNWTDGGGTVNAVNGSGGNYSVNWSNTGNFVVGKGWTTGSPFRTINYNAGVWAPNGNGYLTLYGWTRSPLIEYYVVDSWGTYRPTGTYKGTVKSDGGTYDIYTTTRYNAPSIDGDNTTFTQYWSVRQSKRPTGSNAAITFSNHVNAWKSHGMNLGSNWAYQVLATEGYKSSGSSNVTVW

SEQ ID NO: 89 - protein, 264 aa, Penicillium funiculosum
LPAAEPVSFDIRDENITLARRAEAINYNQDYIASGANVQYSPNMAAGSFSINYNTQGDFVVGLGWQPGDANPITYSGSFSASGVGILAVYGWSTNPLVEYYVMEVHDGYQTAGTHKGTVTTDGGTYDIWEHQQVNQPSILGTSTFNQYISIRQSPRTSGTVTVQNHFNAWAQAGMNLGTLNYQVMAVESWSGSGSGQISLSKGTGGGSTTTTPTGPTSTSTAPSSGGTGAAQWGQCGGIGWTGPTTCVAPYTCKYENAYYSQCQ

SEQ ID NO: 90 - protein, 195 aa, Thermomyces lanuginosus
RQTTPNSEGWHDGYYYSWWSDGGAQATYTNLEGGTYEISWGDGGNLVGGKGWNPGLNARAIHFEGVYQPNGNSYLAVYGWTRNPLVEYYIVENFGTYDPSSGATDLGTVECDGSIYRLGKTTRVNAPSIDGTQTFDQYWSVRQDKRTSGTVQTGCHFDAWARAGLNVNGDHYYQIVATEGYFSSGYARITVADVG

SEQ ID NO: 91 - protein, 203 aa, Dictyoglomus thermophilum
QTSITLTSNASGTFDGYYYELWKDTGNTTMTVYTQGRFSCQWSNINNALFRTGKKYNQNWQSLGTIRITYSATYNPNGNSYLCIYGWSTNPLVEFYIVESWGNWRPPGATSLGQVTIDGGTYDIYRTTRVNQPSIVGTATFDQYWSVRTSKRTSGTVTVTDHFRAWANRGLNLGTIDQITLCVEGYQSSGSANITQNTFSQGS

SEQ ID NO: 92 - protein, 182 aa, Paenibacillus Pabuli
TDYWQNWTDGGGTVNAVNGSGGNYSVNWQNTGNFVVGKGWTYGTPNRVVNYNAGVFSPSGNGYLTFYGWTRNALIEYYVVDNWGTYRPTGTYKGTVTSDGGTYDIYTTMRYNQPSIDGYSTFPQYWSVRQSKRPIGVNSQITFQNHVNAWASKGMYLGNSWSYQVMATEGYQSSGSSNVTVW

SEQ ID NO: 93 - protein, 183 aa, Geobacillus stearothermophilus
ATDYWQYWTDGGGMVNAVNGPGGNYSVTWQNTGNFVVGKGWTVGSPNRVINYNAGIWEPSGNGYLTLYGWTRNALIEYYVVDSWGTYRPTGNYKGTVNSDGGTYDIYTTMRYNAPSIDGTQTFQQFWSVRQSKRPTGSNVSITFSNHVNAWRSKGMNLGSSWAYQVLATEGYQSSGRSNVTVW

SEQ ID NO: 94 - protein, 299 aa, Streptomyces beijiangensis
DTVVNSNQTGTNNGYYYSHWSDGGGSVSMTLGSGGNYGYQWSNVGNFVGGKGWSTGGRKSVNYSGSFNPSGNAYLALYGWTTNPLVEYYVVENFGTYRPTGTFKGTVTSDGGTYDIYETTRVNQPSIEGTKTFKQYWSVRQSKRTGGTITTGNHFDAWSSHGMSMGSFNYMIMATEGYQSSGSSNITVSEGSSGGGTGGGGTGGGTGGGGSGGCTATLSAGDKWSDRYNLNVSVSGAGNWTVTMKVPSPEKVLSTWNVSAAYPDSQTLVAKSNGSGSNWGATIQTNGSWTWPTVTCSAG

SEQ ID NO: 95 - protein, 188 aa, Fusarium oxysporum
TQPTTGTSGGYYFSFWTDTPNSVTYTNGNGGQFSMQWSGNGNHVGGKGWMPGTSRTIKYSGSYNPNGNSYLAVYGWTRNPLIEYYIVENFGTYNPSSGGQKKGEVNVDGSVYDIYVSTRVNAPSIDGNKTFQQYWSVRRNKRSSGSVNTGAHFQAWKNVGLNLGTHDYQILAVEGYYSSGSASMTVSQ

SEQ ID NO: 96 - protein, 189 aa, Aspergillus clavatus
AGTPSSTGWNNGYYYSFWTDNGGTVNYQNGNGGSYSVQWKDTGNFVGGKGWNPGSARTINYSGSFNPSGNAYLTVYGWTTNPLVEYYIVENYGTYNPGNGGTYRGSVYSDGANYNIYTATRYNAPSIEGDKTFTQYWSVRQSKRTGGTVTTANHFNAWAQLGMSLGTHNYQIVATEGYQSSGSSSITVY

SEQ ID NO: 97 - protein, 537 aa, Paenibacillus illinoisensis
WVGMPMGKLHVSGKNLVNSNNQPVLLNGWHQPSGAYWTYQSSNYYLNLHGNNRHAATLAYLKDITDTFADTSPKYGSNHGWNMNQVRLFIDREDMGDVAAGTYNFAGVQTVTQNVIIPYIQYAKTKGVYVVLGLDFTLKDDQATTASNLQKFNQIWGYLASRPEIKSADNVHFELINEPVKSYANGHWGGYNGENDFVDHWNDLRNFQNSIISTIRSQGADNVIWAAGLGYNQFYSLTASHPLTDPLNNYGYAVHWYPGYGAYDNFSILQDQWNTNVKAAADKYPINITEVTWFKNKPGDSAYWNLFNGSNEGFGTNTKTIFNAAGNVSIAAHMNGFILEPGQRSSFADPTAGLKWDGDASRSAMGRFLFNWYHERAQSYPGGNGGGPTTGLVSGATYKIVARHSNKVIDVPGGQNQNNLQLQQWSDLGGNPQKWVLTSIGGGSYTLTSVNSPDKVIDIRNGTLTNGEAVQLMSNLNTTAQHFKVNDLGNGYWSIINVNSNKAIEVENASTSDGAKLQQNTYTGATNQQWKFIAVSN

SEQ ID NO: 98 - protein, 547 aa, Paenibacillus sp-18054
LTVPPGAPAEAWSGMPTPKLHVSGNQLVNANGQPVLLSGWHQPSGSYWTYQSSSYYLDRNGGNRHAANLAYLKDITDTFTDTSPKYGNNHGWYMNQVRLFIDREDMGDVAEGTYNFAGLQAVTQNVIIPYINYARTKGLYVTLGLDFTLKDNQATTQANLDKFNQIWSYLASRPEIRSADNVMFEIINEPVLSYADGRWGGHPSDPHFIAFWNDLRSFQNSIISSIRAQGADNVIWAAGLGWDQYYQLCASHPLTDPLNNVGYAVHWYPGYGAGDNYSVLQQQWDTNIKPCADNYPINITETTWFKRLPGDSDYWNLFNGSSEGFGKNTKAIFTAAGNASIAVHMNGFLLAPGARSSFADPTAGLLYDGNTARDGMARFIFEWYYERAQFLPWNGIWNGLFTGSTYKFVNRATGKNMDVPGGQNNNNLQLNQWTDNGATAQRWVVDDMGTFNNIYRMKSVSSSDGKVMDVRNGTKNNGEAIQLMQDFSNTAQRFRIIRLSNGYWSIINVNSNKAVEVAGGASHDGALLQQNMYRGDHHQQWQLVQIQ

SEQ ID NO: 99 - protein, 598 aa, elephant dung metagenome
WRGMRMPELFIKGRYLMAKDMNGNDSIVNLHGFGQTYSAYFNGYAWCKNPDGSVNWGKTKDAAACVKWNKEQIGLMLDHGWKVNWLRLHMDPAWSNNETKVNQWQSQHPGTYYSENLIVAFDMNLFKKYLDEIFIPMAEYAIENGIYVVMRPPGVCPQKLTVGDEYQQYLIKVWTYVCSHEKLKNNPYIMFELANEPIDMNDGNGNYTSWSDGSQKNCTKFFQKIVDEIRAVGCNNILWVPGLAYQQNYQGYVKYPIVGENIGFAVHCYPGWYGSDSEVASAEQQIVTNGNTYADFQSGWSASIDGVSKLRPIIVTEMDWAPKKYNSSWGKATTGKLGGVGFGNNFKYIMDKTGNVSWMLFTDADKLAKYDDSKADGSTFLTDPEACPRPVYRWYKEYAEPGWKFVETLADEFYMFPGTNSIFSPNIWEKGTLTKNDDGSRTLVTGQYGFGGWKFGGGLDMSGYKYLVLNLTKAPASNQWSLRLFDVDNYWTDPYMKDVKSSTRVVVDLQNMKNSKGVKVDPSHIYILGLWSTGGTPITIKDIYLTNNSDYSPESTGISETLAEKRLDTPIYNLSGQRVTEPRNGHVYIRNGKKFIYK

SEQ ID NO: 100 - protein, 550 aa, Chryseobacterium sp-10696
DEKNLLEDPDSNLSAGASARALAATPMLHVGGRYLKDPCDNNVVLHGVAITPSPWFNGCQYGANSGYCTWDNYNVQGALNYNKAVMNKLTSAADGWYLNYIRLHIDPYWTNDPGPAIPENDISRFNYNRLVTYTDQVIIPLINHARSLGMYVILRPPGVCPNRIAVNDAYHSYLKTVWTFLSQHPGLKNADNVMFELANEPVEILGTNGTWGSTGNEHFAALKNFFQPLVNIIRNNGANNVCWIPGTGWQSHYQGYVNNQITGGNIGYAVHIYPAYWGGLSNYQAFQNAWNINVKPIADIAPIAITETDWAPQGYGTFGIGTTGTAGGSGFGANLKYIVDQSGNVSWNVLAPDNLLHKGDPNAGTAYNNDWEACAAPVKQWFQQYASSNYPVGNCNTTSSLVNNGIYEIEFQTDANKVVDLKSGEDANGAVLRPWTRNGAAAQRWVAIDAGNGYWRFVSKASATNRCIDLASNSNTLGTSIRLWQNYGNDAQAWQVVAVSNGYYKILSKVDPTRGWDIPNCTMDGNSNLHLWDYYGTSCQLFKFKYIGMN

SEQ ID NO: 101 - protein, 828 aa, elephant dung metagenome
FGQFGVVQPLHVNGNQFNDPYGNKVVLHGVMDTPSPYFNSWRWGYSCNDGAVSGCLNYFEKIFTAITDTKQGAYCNLFRLHLDPCWTNDPNKTATGSETGEANISRFSNDRLVKYLNTVYWPIAQKALNHGMYVIMRPPGVCPDNLRVGDAYQNYLKTVWNTVTKNQNVIKNSGVVMIELANEPIHIYNQYGQASATAMRDYFQPVIDVIRKNGYTGIILVPGTGWQSNYKDYDKYPVNDNNYGYAVHDYPGWYSTSDQSYDHNTAIRAFGDNVPVVKKKPIVITEVDWSPEKAGTGHYNEHGQWVTSNYGTWATASTTKWGYAYKAVLDYYDNISMTLSGTDTYIDVDTYLNSGKVTGAFGNIWEACGAACMYWYKLWAQKDYAHATGSSSSSTTTQPSTGGSTSTGSTTTGNGEFTNVDLSSSLIPGWSQGGVRPNGWACNDAGDYPASGEAGSGPRVMQFNGGGDFKYGFYFRQQDASKPGYIQYGTTSGYELALNSFGNYCLTFNCAAWAGSPYVKAEVITSSGNVIASTIVKCNLNLNKNTNASTAKSDQGYLSFYVLNKGNFSVKFTPCADANGGSGTWVEAVVGNINLRYMGNPLSFEKANYVNPGWKILDGGKIVETGDAGSGPRIFNFPAGGQFNYGLYIRSTSTELSENYAEFGSRWGYGLNFLPGRYTLTYNCAAWTGSPYVKCEVINENDQVVASSITNCSLNLNKNLNASTANAPQGSVSFTVSNTGSYKLRWTPVANANGDAGYWVEAVIGHIKITQNSGNKVAAYTDDNDEVTSVAEVENEANSEETFNLSGQRVENPTKGIYIKNGKKVIVR

SEQ ID NO: 102 - protein, 577 aa, elephant dung metagenome
ETLSPLHVEGRWLCNENGEHVNLHGFGQTYSPWFNEQGNGWGWGYSVSDCMKYNKGLIDKIISAGWKMNWLRLHMDPHWSNTPGVSTSGENDISAFNESRFRTYLNSLFIPMAEYANQHGMYVVMRPPGVCPEKIEIGGAYYKYLLKVWGIVASNEKLKNNPGIMFELANEPINIKGTDGQYTSYSDGSNQNMTKFFQAIVDSIRSCGANNILWVPGLGYQSQYAGFVKYPIQGENIGYAVHCYPGWYGSDSEADGGSVEQGVVTKGAGYSEFKAGWDNQVGPVAKIAPILITEMDWAPKSYNCSWGKATTGVAGGVGFGANFKYIVDKTGNVSWMLFTGPEHLAKYVDSKADGKTFLTDPEACVRPIYRWFKEYTDPNWVFEDTLASQTMYFPQTDTWINPSIWEKGSFDATTGALVTGQYGFGGWQFGTGLDLSKWKYLVIKLKQTASSGGWSFRVFDENSYWSNCYQKSVGSSLRAIVPLNGMYKTIDGSVSNSSKVNPSHIYIAGFWSYGGTPLYIDKLYLTNQSDYSEEVGLEQVAQDDDVPIVMFDVMGRRVETPHRGGFYMSKDRKFIVR

SEQ ID NO: 103 - protein, 537 aa, Paenibacillus campinasensis
AWSGMPMSKLRVSGNQLVNSEGQPVVLSGWHQPGGSYWTYQGSNYYLNRNGGNRHAAILEYLKDITDTFSDTSPKYGNNHGWYMNQVRLFIDREDMGDVAAGTYNFAGVQSFTQNVVVPYIQYAKSKGIYVTLGLDFTLQDNQATTQANLEKFNQIWGYLAAQPAIKSADNVMFEIINEPVLSYANGRWGGHPSDPDFPAFWDSLRNFQNSIISTIRSQGADNVIWAAGLGWDQYYQLCATRPLTDPLNNIGYAVHWYPGYGAKDNGSILQQQWDTNIKPCADNYPINITETTWFKRLPGDSEYWELFNGSNEGFGKNTKAIFTTAGNVSIAVHMNGFLLQPGPRSTFADPTSGLLFDGDPARDGMARFIFEWYYERAQLHPWNGIWNGIISGETYKIVNRASGKVIDVPGGQNSNSLQLQQWPDNNATAQRWVADDMGAYNNVYRLRSVSSSDGKVMDVRNGSSNNGEAIQLMQDYGNQAQRFRLIKLSNGYWSILNANSNKAVEVAGGSSADGAKLQQNPYRGDLHQQWQLVKVN

SEQ ID NO: 104 - protein, 536 aa, Paenibacillus sp-62250
WEGTPVSKLHVSGNQLVNGSGQPVLLSGWHQPTGAYWTYQNSNYYLNRNAGNRHKATLEYLKEITDTFTSTSGKYGNSHGWYSNQVRLFIDREDMGDVAAGTYNFAGLQAATQNVIIPYVQYAKTKGLYVTLGLDFTLLDNKATTQANLDKFNQIWGYLASQPALKSADNVMFELVNEPVLSDVNGQWGGNPSQPNFAAFWNSLKNFQNSMISTIRSKGADNVIWAAGLGWDQHYQLTASNPLTDPLNNYGYAVHWYPGYGAYDNFNSLQQIWDSSIKPAADKYPINITETTWFKRQPGDSEYWDLFNGTNAGFGKNTKAIFTAAGNVSIAVHMNGFLLDPGPKSSFADPDGGLMYDGNATRDGMARFIFEWYYERAQFNPWNGVWNGVTNNAKYKLINRASGKAIDVPNGQNTNSLQLQQWPENTAAAQQWTATDMGTYNNVYRLRSVNSSDNKVMDVRNGTKNNGEAIQLMQDLNNTAQQFRLIKLSNGYWSILNVNSNRAVEVTGASTADGAKLQQNQYRGDLHQQWKLVQVN

SEQ ID NO: 105 - protein, 536 aa, Paenibacillus favisporus
WNGMPMSKLHVSGNQLVNSSGQPVLLSGWHQPSGSYWTYQSSSYYLDRNGGNRHAAILDYLKVITDTFTGTSAKYGSSHGWYMNQVRLFIDREDMGDVAAGTYNFAGLQSVTQDVIIPYIAYAKTKGLYVTLGLDFTLSNNQATTPSNLNKFNEIWGYLASQPAIKSADNVMFELINEPVLSDVNGQWGGNPSQPNFADYWNSLKKFQNSIISTIRSKGADNVIWAAGLGWDQYYQLCASSPLTDPLNNIGYSVHWYPGYGAHDDYATLQQQWDTNIKPCADHYPINITETTWFKTQPGDSSYWELFNGSNEGFGKNTKAIFTAAGNVSMTAHMNGFLLEPGTRSSFADPTAGLKYDGNTARDGMARFLFEWYYERAQLNPWNGIWNGVLSGSTYKIVNRASGKAIDVPGGQNTNGLQLQQWSDNNATAQQWVADDLGTYNNVYRLRSVSSADQKVMDIRNGTKNNGEAVQLMQDLGNTAQQFRLIKLSNGYWSILNVNSNKAVEVAGASSADGAKLQQNLYRGDLNQQWQLVKIN

SEQ ID NO: 106 - protein, 535 aa, Paenibacillus tundrae
WVGMPMGKLHVSGKNLVNSSNQPVVMSGWHQPSGAYWTYQNSNYYLTRNGNNRHAATLAYLKDITDTFTSTSPKYGSSHGWNMNQIRLFIDREDMGDVAAGTYNFAGIQTVTQNVIIPYIQYAKTKGVYVTLGLDFTLKDDQATTAANLQKFNQIWGYLASRPEIKSADNVHFELINEPVKSYANGHWGGYNGENDFVDHWNDLRNFQNSIISTIRSQGADNVIWAAGLGYNQFYSLTASHPLTDPLNNYGYAVHWYPGYGAHDNMSILQDQWNTNVKAAADKYPINITEVTWFKTKPGDSEYWNLFNGSNEGFGNNTKTIFNAAGNVSIAAHMNGFILDAGERSSFADPTAGLKWDGDASRSAMGRFLFDWFYERAQSYPNGGTPSTGLTPGATYKIVARHSGKVIDVPGGVNESNLQLQQWSDLGGNPQKWVLTQIASGIYSLTSVNSPDKVIDIRNGTSNNGEAVQLMSNLNTTAQHFKINDLGNGYWSIINVNSNKAIEVAGSSTADGAKLQQNGFTNATNQQWKFVAVNN

SEQ ID NO: 107 - protein, 536 aa, Paenibacillus sp-62603
WVGMPMSKLHVSGNQLVNSSGQPVLLSGWHQPSGAYWTYQNSNYYLNQNGGNRHAAILAYLKDITDTFTSTSAKYGNNHGWYMNQVRLFIDREDMGDVAAGTYNFAGLQAVTQNVIIPYINYAKTKGLYVTLGLDFTLSNNQATTPANLAKFNQIWGYLADQPGIKSADNVMFELINEPVLSDVNGVWGGHPSQSNFIAYWNSLKNFQNSMISTIRSKGADNVIWASGLGWDQYYQLCAAYPLTDSLNNIGYSVHWYPGYGAYDDYTPLQQQWDTNIKPCADNYPINITETTWFKTQPGDSSYWDLFNGSNAGFGKNTKAIFTAAGNVSIAVHMNGFLLNPGTRSSFADPTGGLMYDGNTARDGMARFIFEWYYERAQFNPWNGIWNGVTNGATYKLVNRASSKILEVPGGQNTNALQLQQRADNNTTAQRWIVTDQGTYNNYYELRSVSSSDNKVMDVRNGTKNNGEAIQLMQDLSNTAQQFRLIKLSNGYWSILNVNSNKAIEVTGSSTADGANIQQNLYSGNLNQQWQLVQIN

SEQ ID NO: 108 - protein, 536 aa, Paenibacillus sp-62332
WSGMPMSKLRVSGNQLVNSSNQPVLLSGWHQPTGSYWTYQNSNYYLNRNGGNRHAAILEYLKDITDTFTSTSPKYGNNHGWYMNQVRLFIDRQDMGDVAAGTYNFAGLQSVTQNVIIPYIDYARTKGLYVTLGLDFTLLNDEATTQSNLDKFNQIWGYLAAQPGIKSADNVMFEIINEPVKSYANGRWGGHPSDPDFVAFWNSLRTFQNAIISTIRNQGADNVIWAAGLGWDQYYQLCASHPLTDPLNNLGYAVHWYPGYGANDNNSVLQQQWNTNIKPCADHFPINITETTWFKWLPGDSSYWNLFNGSNEGFGKNTKAIFTAAGNVSIAVHMNGFLLEPGTRSSFADPTAGLKFDGNAARDGMARFIFEWYYERAQMYPWNGIWNGISSGTTYKIVNRHSGKVIDVPGGQNNNALQLQQWADLHTSAQRWVVDDMGSSTNVYRLRSVSSSDNKVMDVRNGTINNGEAIQLMQDFGNTAQRFRLIRLSNGYWSIININSNKAVEVSGSSTADGANLQQNPYRGDHHQQWQLIAVN

SEQ ID NO: 109 - protein, 536 aa, Paenibacillus sp-62248
WTGMPMSKLRVSGNQLVNGSGQPVLLSGWHQPTGAYWTYQNSSYYLNQQGGNRHAAILAYLKDITDTFTSTSAKYGNNHGWYMNQVRLFIDREDMGDVAAGTYNFAGLQAVTQNVIIPYIAYAKTKGLYVTLGLDFTLQNNQATTTANLDKFNQIWGYLASQPAIKSADNVMFELVNEPVLSYANGGWGGNPIESDFVAHWNALRDFQNSMIATIRGKGADNVIWAAGLGWDQYYQLCATNPLTDPLNNVGYSVHWYPGYGANDNYSVLQQQWDTNIKPCADAYPINITETTWFKWKSGDSEYWKLFDGTNAGFGKNTKAIFTAAGNVSIAVHMNGFLLNPGTRSSFADPTAGLMYDGDASRDGMARFIFEWYYERAQFNPWNGVWNGITSGSTYKLINRASGKAIDVPSGQNTNGLQLQQWTDNNATAQRWVADDLGTYNNVYRLRSVSSSDNKVMDVRNGTKNNGEAIQLWSDLNNTAQKFRLIKLSNGYWSLINVNSNKAVEVAGSSTADGAKLQQNLYRGDLNQQWQLVKIE

SEQ ID NO: 110 - protein, 536 aa, compost metagenome
WSGMPMSKLHVSGKQLVNSSGQPVLLSGWHQPSGPYWTYQNSNYYLSQNGNNRHAAILAYLKDITDTFTDTSAKYGSTHGWYMNQVRLFIDREDMGDVAAGTYNFAGLKSVTQNVIIPYINYAKTKGLYVTLGLDFTLQDNKATTSSNLAKFNEIWGYLASQTGIKSADNVMFELINEPVLSDVNGVWGGNPSQSNFVDYWNSLRNFQNSIISTIRSKGADNVIWAAGLGWDQYYQLCASHPLTDPLNNLGYSVHWYPGYGANDNFSTLQQIWNTTIKPCADNYPINITETTWFKRKAGDSDYWNLFNGSNEGFGKNTKAIFTAAGNVSIAAHMNGFILNPGTRSSFADPTAGLQYDGDATRDGMARFLFEWYYERAQFNPWNGVWNGLTNGGTYKLVNRASGKVIDVPGGQNTNALQLQQYADNGATAQQWVITDQGTYNNFYKLTSVSSSDGKVMDVRNGTSNNGEAIQLMQSFNNTAQQFRLIKLSNGYYCVLNVNSNKAVEVANASTSNGALIQQNWYRGDLNQQWQLVKIN

SEQ ID NO: 111 - protein, 391 aa, Bacillus subtilis
AASDVTVNVSAEKQVIRGFGGMNHPAWAGDLTAAQRETAFGNGQNQLGFSILRIHVDENRNNWYKEVETAKSAVKHGAIVFASPWNPPSDMVETFNRNGDTSAKRLKYNKYAAYAQHLNDFVTFMKNNGVNLYAISVQNEPDYAHEWTWWTPQEILRFMRENAGSINARVIAPESFQYLKNLSDPILNDPQALANMDILGTHLYGTQVSQFPYPLFKQKGAGKDLWMTEVYYPNSDTNSADRWPEALDVSQHIHNAMVEGDFQAYVWWYIRRSYGPMKEDGTISKRGYNMAHFSKFVRPGYVRIDATKNPNANVYVSAYKGDNKVVIVAINKSNTGVNQNFVLQNGSASNVSRWITSSSSNLQPGTNLTVSGNHFWAHLPAQSVTTFVVNR

SEQ ID NO: 112 - protein, 391 aa, Bacillus amyloliquefaciens
AANDVTVNISAERQVIRGFGGMNHPAWVGDLTAAQRETAFGNGQNQLGFSILRIHVDENRNNWYKEVETAKSAIKHGAIVFASPWNPPNDMVETFNHNGDTSAKRLRYDKYAAYAQHLNDFVNFMKSNGVNLYAISIQNEPDYAHEWTWWTPQEILRFMRENAGSINARVIAPESFQYLKNLSDPILNDPQALANMDILGTHLYGTQVSQFPYPLFKQKGAGKDLWMTEVYYPNSDNNSADRWPEALDVSQHIHSSMVEGDLQAYVWWYIRRSYGPMKEDGTISKRGYNMAHFSKFVRPGYVRIDATKNPNPNVYVSAYKGDNKVVIVAINKTNTGVNQNFVLQNGSASQVSRWITSSNSNLQPGTDLKVTDNHFWAHLPAQSVTTFVVKR

SEQ ID NO: 113 - protein, 392 aa, Bacillus licheniformis
AASDATVRLSAEKQVIRGFGGMNHPAWIGDLTAAQRETAFGNGQNQLGFSILRIHVDENRNNWYREVETAKSAIKHGAIVFASPWNPPSDMVETFNRNGDTSAKRLRYDKYAAYAKHLNDFVTFMKNNGVNLYAISVQNEPDYAHDWTWWTPQEILRFMKENAGSINARVIAPESFQYLKNISDPIVNDPKALANMDILGAHLYGTQLNNFAYPLFKQKGAGKDLWMTEVYYPNSDNHSADRWPEALDVSHHIHNSMVEGDFQAYVWWYIRRSYGPMKEDGTISKRGYNMAHFSKFVRPGYVRVDATKSPASNVYVSAYKGDNKVVIVAINKNNSGVNQNFVLQNGSVSQVSRWITSSSSNLQPGTNLNVTDNHFWAHLPAQSVTTFVANLR

SEQ ID NO: 114 - protein, 391 aa, Bacillus subtilis
AANDVTVNISAEKQVIRGFGGMNHPAWVGDLTAAQRETAFGNGQNQLGFSILRIHVDENRNNWYKEVETAKSAIKHGAIVFASPWNPPSNMVETFNHNGDTSAKRLRYDKYAAYAQHLNDFVTFMKSNGVNLYAISIQNEPDYAHEWTWWTPQEILRFMRENAGSINARVIAPESFQYLKNLSDPILNDPQALANMDILGTHLYGTQVSQFPYPLFKQKGAGKDLWMTEVYYPNSDNNSADRWPEALDVSQHIHNSMVEGDFQAYVWWYIRRSYGPMKEDGTISKRGYNMAHFSKFVRPGYVRIDATKNPNPNVYVSAYKGDNKVVIVAINKSNTGVNQNFVLQNGSASQVSRWITSSNSNLQPGTNLKVTDNHFWAHLPAQSVTTFVVIR

SEQ ID NO: 115 - protein, 393 aa, Paenibacillus pabuli
AASDVTVNLSSEKQLIKGFGGINHPNWIGDLTPSQRDTAFGNGQNQLGFSILRIYIDDNKNNWYKEIPTAKRAIEQGAIVFASPWNPPSDMVETFNRNGDTAAKRLKYDKYAAYAQHLNDFVSYMKSNGVNLYAISVQNEPDYAHDWTWWTPQEMLRFMKDYAGSITGTKVMAPESFSYLKEMSDPILNDPQALANMDILGAHTYGTQFSNFPYPLFKQKGAGKELWMSEVYYPNSNANSADHWPEALDVSYHIHHAMVEADFQAYVWWYIRRQYGPMKEDGTISKRGYNMAHFSKFVRPGFVRVDATKNPDTQTFISAFKGDNKVVIVAINRGTSAVNQKFVLQNGNASNVSSWVTDSTRNLAAGSSIIMTGNTFTAQLPSQSVTTFVAQLN

SEQ ID NO: 116 - protein, 391 aa, Bacillus amyloliquefaciens HB-26
AASDATVNISAERQVIRGFGGMNHPAWIGDLTAPQRVTAFGNGQNQLGFSVLRIHVDENRNNWYKEVETAKSAIKHGAIVFASPWNPPNDMVETFNHNGDTSAKRLRYDKYAAYAQHLNDFVNFMKSNGVNLYAISMQNEPDYAHEWTWWTPQEILRFMRENAGSINTRVIAPESFQYLKNISDPILNDPQALRNMDILGTHLYGTQVSQFPYPLFKQKGGGKELWMTEVYYPNSDNYSADRWPEALGVSEHIHHSMVEGDFQAYVWWYIRRSYGPMKEDGMISKRGYNMAHFSKFVRPGYVRIDATKNPEPNVYVSAYKGDNKVVIVAINKNNTGVNQNFVLQNGTASQVSRWITSSSSNLQPGTDLKVTDNHFWAHLPAQSVTTFVVKR

SEQ ID NO: 117 - protein, 382 aa, Pseudoalteromonas tetraodonis
SNVTINFNTQYQQIDGFGGMNAPGWINDLTPAQATKAFGTGNGEMGLSIMRMRIAPDSNQWYKQVPTAKIAKSYGAKLLASPWSPPAYMKSNNNLNNGGKLEKTHYWGYTNHLMDFTNYMASQGASVYALSLQNEPDWHPEYESCDWSASDFVNYLNDQGWRLDPALKILAPESLGFNKALSDPILNNSVANNYVDIIGGHLYGVSPSNYPLALQKGKKLWMTEHYTDNEDGNNWNASIDVGLELHQSMVSNYSAYIWWYIRRSYGLMSEDGNVSKRGYIMAQFSKYIRPGYTRIGATEMPENNVYVTAYKNNAGKLVIVVVNKSGSPKALDFTLQNGTVNTLTKYSTSASMNMEYRGKSTVSNNRFSAYADAWAVQTFVSN

SEQ ID NO: 118 - protein, 391 aa, Paenibacillus sp-19179
ASDAIVNISAEKQVIRGFGGINHPVWIGDLTAAQRETAFGNGNNQLGFSILRIYVHEDRNQWYREVETAKRAIALGAIVFASPWNPPADMVETFNRNGDPSAKRLRYDKYAAYAQHLNDFVTYMRNNGVNLYAISVQNEPDYAHDWTWWTPQEMLRFMKENAGSINTRVIAPESFQYLKNMSDPILNDPQALANMDILGAHLYGTQVSNFAYPLFKQKGAGKDLWMTEVYYPNSDNNSADRWPEALDVSYHIHNAMVEGDFQAYVWWYIRRSYGPMKEDGTISKRGYAMAHFSKFVRPGYVRVEATKNPETNVYVSAYKGNKKLVIVAVNKNNSGVNQNFVLPNASVSKISRWITSGSSNLQPGTELTMTGGNFWAHLPAQSVTTFVADLG

SEQ ID NO: 119 - protein, 383 aa, Pectobacterium carotovorum subsp. carotovorum
DTVKIDAKTSYQIIQGFGGMNAPGWINDLTTEQVNTAFGNDTGQIGLSIMRMRIDPDANRWNIQVSSARQASLLGAKLMATPWTPPAYMKSNKSLINGGRLLSEHYSGYTEHLLKFSNFMQTNNAPLYAISIQNEPDWKPDYESCEWNGNDFKNYLKSQGSKFGSLKVIVGESLNFNHSLTDPTLNDSEAAKHVAIVGGHLYGTTPKPYPLAQNKGKEVWMTEHLVDSKQSANNWSSALEVASEMNASMVANYNAYVWWYIRRSYGLLTEDGKVSKRGYVMAQYAKFVRPGFQRIQATENPQANVHLTAYKNSEGKMVIVAINTNDSDQLLSLNISNHTVSKFEKYSTSAILNVEYGGTYKVDSNGKSSVWLNPLSVTTFVGK

SEQ ID NO: 120 - protein, 565 aa, Ruminococcus sp. CAG330
ADVCVIDTDTEHQMIRGFGGINHPEWAGDLTQAQRQTAFGNGENELGLTVLRVFVNPDSSQWSRALPTAQFATQMGVTVFASPWEPPANLTESGGSNGKLHLPKSNYAAYAKHLNDFGTYMKNNNVDLYAISVQNEPDYASEWTYWSTDETTDFIANYGDQITSTRLMSPESFQYAPENASWVSDGGKKFYRKILNNSKAMANCDVFGTHFYGTQRSWMDFPDLENSGKEIWMTEVYVPNSDKDSANRYPEALQVSENIHNAMVVGNMSAYTWWYIRRNYGLMTEDGKISKRGYCMAQYSKYVRPGDVRIDATEQPADNVYVSAYKGDDNQVTIVAINKGTESYSQQFAVDADAQITEVDRYRTSASENLAKTENMEHDSSSFWAQLPAESVSTFVVTLEDQPVEPDENGYYFHDTFESDNCDWQGHGSADITLSGRIPYQGTNALLVQNRASAWNGAEKVLPAKAFQAGKEYSFSVCLNYMDGESSKNAALSLQYTDAAGETKYARIASASAAKGNYVQLANPSFKLPDGGKNFKIYVETEGDTDNFYIDEAIGAVKGTAIEGP

SEQ ID NO: 121 - protein, 396 aa, Streptomyces sp-62627
SRTPSAAATSVTVDPSATRQTIRGFGGMNHPLWIGDLTPAQRDTAFGNGEGQLGFSVLRIPVSEDRANWSREVATAKRATELGAIVFASPWNPPANMVETFVRGQQTDAKRLRHSMYGAYAQHLNDFVAFMKSNGVNLYAISVQNEPDYAHDWTWWTPSEMTRFLRENAGSISTKVIAPESFQYVKTFSDPILNDAAALANLDILGAHLYGTSFQNFPYPLFKQKGGGKELWMTEVYHPNSSDSADLWPQALDVAEHIHRAMVDAEFQAYVWWYIRRGYGPMREDGRISKRGAGMAHFSKFVRPGHVRVAVTPAPQPNVYLSAYKGGGSRVVVVAVNKGASPVSQQFTLNNNNSSGVSSWVTDASRNLASQGRITVANGAFTARLPAQSVTTLVTG

SEQ ID NO: 122 - protein, 392 aa, Clostridium saccharobutylicum
ASNATINLSAQKQVIRGFGGINLPAWAGDLTAAQRETAFGNGDNQLGLSVLRIYVDDNKNNWYKELATAKKAIEHGAIVFATPWNPPAYMTEKFNRNGDTNAKRLRYDKYAAYAQHLNDFVSYMKNNGVNLYAISVQNEPDYGKEWTWWTPQEILRFIKENAGSINCRVMSPESFSYQKNMYDPILNNPQALANMDILGTHTYGTRVNDFAYPLFKQKGAGKELWMTEVYVPNSDTNSADRWPEALDVADHINNAMVEGDFQAYVWWYIRRSYGFIKEDGNVSKRGYMMAHFSKFVRPGYVRVDATKNPTPNVYLSAYKGNNKVVIIAINKGTSDVKQSFTMPNSKVSSVSSWQTTATANLAKSASNTNVYNGNFTATLPAQSVTTFVGDIK

SEQ ID NO: 123 - protein, 413 aa, Paenibacillus panacisoli
ASDAVINLSAQKQVIRGFGGINHPAWIGDLTAAQRETAFGNGQNQLGFSILRVYIDPDRNNWSREVATAKKAIEKGALVFASPWNPPSSMVETFNRNGDRNAKRLRYDKYAAYAQHLNDFVTYMKNNGVNLYAISVQNEPDYAHEWTWWTPQEILRFMKENAGSINCKVMAPESFQYLKNISDPILNDPQALANMDILGAHLYGTQVSNFAYPLFKQKGAGKELWMTEVYYPNSDNNSADRWPEALEVSHHMHNAMVEGDFQAYVWWYIRRSYGPMKEDGTISKRGYNMAHFSKFIRPGYVRVDATKNPDTNVHVSAYKGNNKVVIVAINRGTTAVNQNFVLQNGRAATLSRWITDANRNLAPESNLNASSGSFFAHLPAKSVTTFVGDLTGSTLNIEDAALTNSVTQDTYSK

SEQ ID NO: 124 - protein, 398 aa, Human Stool metagenome
AQAASDAVINLNNTHQEIMGFGGMNHPTWAGDLTSSQRETAFGNGTNQLGFQVLRIWVDSDRNNWYKELATAKAALAKGAIVFATPWNPPSNLCETFYKNGSANAKRLKHDKYAAYAQHLNDFVTYMRNNGVELYGISVCNEPDYGHDWTWWTESEVVTFLKYYAGSINCRIIAPESFSYQKSYYDAIINDSQALAQVDIIGTHLYGTSYNNFSYPLYHQKASSKQLWMTEVYTPNSTSSADKWPEAINVAEHIHKAMVNDFQTYVWWYIRRSYGPMKEDGTLSKRGYCMAQFSKFIRRGYKRVDATENPNNGVYVSAYTGDGKAVIVAVNSGSSDCSQSFTIKGKTLKNVDRYRTSGSENLAKTSNLELSGNGFWAYLPANSVSTFVCTVENSSSNP

SEQ ID NO: 125 - protein, 372 aa, Vibrio rhizosphaerae
YQEIDGFGAMNAPGWVNDLTSAQATKAFGNGDGQMGLSIMRMRIDPDSNQWYRQVPTAQIAYSYGAKLLATPWSPPAYMKTNNNVNNGGKLKKEHYWGYTDHLMDFTNYMAGKNAPIYALSIQNEPDWHPNYESCDWSGADFVNYLNDQGWRLDSSLKILAPESLGFNPALSDPILKDSVASSHIDIIGGHLYGVQPRNYPLALQKGKKLWMTEHYTDTDNANIWDKAMNVGLELHQSMVSNYSAYIWWYLRRSYGMLTEDGNISKRGYIMSQFSKFIRPGDVRIAATEVPESNVYVTAYKNRSGKLVIAVVNKTNSHKKLDFTLQNGTVGSMTKYVTSASQNVGYAGKYAVSNNRFTAYADPLSVQTFVSE

SEQ ID NO: 126 - protein, 557 aa, Clostridium acetobutylicum
ASNVMVNLASKKQVIRGFGGMNSVAWAGDLTAAQRETAFGNGNNQLGLSVVRIFVDDNKNNWYKELPTAKSAIAHGSIVFATPWNPPSSMTETFNRNGEKAKRLRYDKYGDYAKYLNDFVSYMKNNGVNLYAISVQNEPDYGRDWTWWTPQEVLRFMRDYAGSINCRVMSPESFSYQKNMYDPILNDPKALANMDILGTHTYGTQVKDFPYPLFKQKAAGKDLWMTEVYVPNSDANSADRWPEALEVANHINNAMVEGDFQAYVWWYIRRSYGLIKENGAISKRGYMMAHFSKFVRPGYVRVDATKNPVGNVYVSAYTGNNKVVIVAINKGTYPVNQSFNIQNSTVSNVSSWVTSGTLNMAKTNSNINAANGRFNASLPAQSVTTFVADLNSTKPTTNPTTNPTPGSTVTLNNGWYYIKNINAQKYLQVANNTGKAGQNVELGSGSGVAGQKWYLTNTGDGYITLKNALGNYMLDVSYGENKDGSNIQIFNAYSGDSQKFAVKASSKNGQYSVATKSSNGSKVLDDYNFGTADGTNVCQWTYGGNANQLWVFEPTNN